SEQUENCE LISTING (40 sequences)

SEQ ID NO: 1 - protein, 333 aa, Mycobacterium tuberculosis
MFYDDDADLSIIQGRKVGVIGYGSQGHAHSLSLRDSGVQVRVGLKQGSRSRPKVEEQGLDVDTPAEVAKWADVVMVLAPDTAQAEIFAGDIEPNLKPGDALFFGHGLNVHFGLIKPPADVAVAMVAPKGPGHLVRRQFVDGKGVPCLVAVEQDPRGDGLALALSYAKAIGGTRAGVIKTTFKDETETDLFGEQTVLCGGTEELVKAGFEVMVEAGYPAELAYFEVLHELKLIVDLMYEGGLARMYYSVSDTAEFGGYLSGPRVIDAGTKERMRDILREIQDGSFVHKLVADVEGGNKQLEELRRQNAEHPIEVVGKKLRDLMSWVDRPITETA

SEQ ID NO: 2 - DNA, 1002 bp, Mycobacterium tuberculosis
atgttctacgacgacgacgcagacctgtcgatcattcagggccgcaaggttggtgtgatcggctacggcagccaggggcacgcgcactcgctaagcctgcgcgactcgggtgtgcaggtgcgcgtcgggctgaagcagggttcgcggtcgcggcccaaggtagaagagcagggcctggacgtcgacactcccgccgaggtcgccaaatgggccgatgtggtcatggtgttggcccccgacaccgcccaggccgagatcttcgcaggagacatcgaacccaacctcaagcccggtgacgcgctgttcttcggtcacggactcaacgttcacttcggcttgatcaagccgcccgccgacgtcgccgtcgcgatggtcgccccgaagggaccgggtcatttggtgcgccgccagttcgtcgacggcaagggtgtgccgtgtttggttgcggtagagcaggatccgcgaggcgacggcttggcgctggcgctgtcgtatgccaaagcgatcggcggcacccgggccggcgtcatcaagacgacgttcaaagacgagaccgaaaccgacctgttcggtgagcaaacggtgttgtgcggcggcaccgaggaattggtcaaggccgggttcgaggtcatggtcgaagccggctaccccgcggaattggcctacttcgaggtgctgcacgagctgaagctgatcgtcgacttgatgtacgagggtggcctggcgcggatgtactactcggtgtcggacaccgcggaattcggcggctacctctcaggcccgcgcgtcatcgatgccggcaccaaggagcggatgcgcgacatcctgcgggagatccaggacggtagctttgtccacaagctggtcgccgacgtcgagggcggcaacaaacagctcgaagagttgcgccggcaaaacgccgagcaccccatcgaggtcgtcggcaagaaactccgcgacctgatgagctgggtggaccgcccgatcaccgagacggcctag

SEQ ID NO: 3 - protein, 140 aa, Mycobacterium tuberculosis
MSSEEKLAAKVSTKASDVASDIGSFIRSQRETAHVSMRQLAERSGVSNPYLSQVERGLRKPSADVLSQIAKALRVSAEVLYVRAGILEPSETSQVRDAIITDTAITERQKQILLDIYASFTHQNEATREECPSDPTPTDD

SEQ ID NO: 4 - DNA, 423 bp, Mycobacterium tuberculosis
atgtcgtcggaggagaagctggccgccaaggtgtccaccaaggcctccgatgtggcttccgacatcggcagcttcatcaggtcgcaacgtgagacggcgcacgtctcgatgcggcagctcgccgagcggtccggcgtcagcaatccgtacctgagccaggttgagcgcggattgcgtaagccgtccgccgacgtgttgagccagatcgcaaaggcgctgcgggtctcggccgaagtcctttatgtgcgcgccgggattctcgagcccagcgagaccagtcaggtgcgtgacgccatcatcaccgatacggcgatcaccgagcgtcagaagcagattctgctcgatatctacgcgtcatttacccaccagaacgaagccacccgggaggagtgtccgagcgatccgacaccgaccgatgactag

SEQ ID NO: 5 - protein, 151 aa, Mycobacterium tuberculosis
MTETTPAPQTPAAPAGPAQSFVLERPIQTVGRRKEAVVRVRLVPGTGKFDLNGRSLEDYFPNKVHQQLIKAPLVTVDRVESFDIFAHLGGGGPSGQAGALRLGIARALILVSPEDRPALKKAGFLTRDPRATERKKYGLKKARKAPQYSKR

SEQ ID NO: 6 - DNA, 456 bp, Mycobacterium tuberculosis
atgaccgaaaccaccccagccccgcaaaccccggcggccccggccgggcccgcacaatcgttcgtgttggagcggcccatccagaccgttgggcgccgtaaggaggccgtggtacgagtgcggctggtgcccggcaccggcaagttcgacctcaacggccgcagcttggaggactacttcccaaacaaggtgcaccagcagttgatcaaggcacccctggtcaccgtggatcgggtggaaagtttcgacatctttgcccacctgggcggcggcggcccgtcgggtcaggccggcgcgctgcgcctgggtatcgcccgggcattgattctggtatcgccggaggaccggcccgcgctgaagaaggccggcttcttgacccgtgatccacgcgccaccgagcgcaagaagtacggcctgaagaaggcccgcaaggcgccccagtacagcaagcgctga

SEQ ID NO: 7 - protein, 226 aa, Mycobacterium tuberculosis
MVLARPDAVFAPARNRCHVSLPVNAMSLKMKVCNHVIMRHHHMHGRRYGRPGGWQQAQQPDASGAAEWFAGRLPEDWFDGDPTVIVDREEITVIGKLPGLESPEEESAARASGRVSRFRDETRPERMTIADEAQNRYGRKVSWGVEVGGERILFTHIAVPVMTRLKQPERQVLDTLVDAGVARSRSDALAWSVKLVGEHTEEWLAKLRTAMSAVDDLRAQGPDLPA

SEQ ID NO: 8 - DNA, 681 bp, Mycobacterium tuberculosis
atggttctcgccaggccggacgctgttttcgcgccggccaggaaccggtgtcacgtttcgctgccggtgaacgcgatgtcattaaagatgaaagtatgtaatcatgtaattatgaggcaccatcacatgcacgggcggcgctacggtcgccccggcggctggcagcaagctcagcaaccagatgccagtggggcggcggaatggttcgctggccgcctgcccgaggactggttcgacggcgaccccaccgtcatcgtcgaccgtgaagaaattacggtgattggcaagctgcctggactcgagagccccgaggaagaaagtgcggcccgagcctcgggccgcgtgtcgcgattccgcgacgaaacccgaccggagcgaatgactatcgccgatgaagcccagaatcgctacggacgcaaggtgtcctggggcgtcgaggtcggtggtgagcgaatcttgttcacgcacatcgcagtaccggtgatgacgcggttaaagcagccggaacggcaggtgctggacaccttggtcgacgctggcgtggctcgttcccgctcggatgccctcgcgtggtcggtcaagctggtcggcgagcacaccgaggagtggctggccaagctgcgcaccgccatgtcggcggtggacgatctgcgcgcgcaaggcccggatcttccggcctaa

SEQ ID NO: 9 - protein, 396 aa, Mycobacterium tuberculosis
MAKAKFQRTKPHVNIGTIGHVDHGKTTLTAAITKVLHDKFPDLNETKAFDQIDNAPEERQRGITINIAHVEYQTDKRHYAHVDAPGHADYIKNMITGAAQMDGAILVVAATDGPMPQTREHVLLARQVGVPYILVALNKADAVDDEELLELVEMEVRELLAAQEFDEDAPVVRVSALKALEGDAKWVASVEELMNAVDESIPDPVRETDKPFLMPVEDVFTITGRGTVVTGRVERGVINVNEEVEIVGIRPSTTKTTVTGVEMFRKLLDQGQAGDNVGLLLRGVKREDVERGQVVTKPGTTTPHTEFEGQVYILSKDEGGRHTPFFNNYRPQFYFRTTDVTGVVTLPEGTEMVMPGDNTNISVKLIQPVAMDEGLRFAIREGGRTVGAGRVTKIIK

SEQ ID NO: 10 - DNA, 1191 bp, Mycobacterium tuberculosis
gtggcgaaggcgaagttccagcggaccaagccccacgtcaacatcgggaccatcggtcacgttgaccacggcaagaccaccctgaccgcggctatcaccaaggtcctgcacgacaaattccccgatctgaacgagacgaaggcattcgaccagatcgacaacgcccccgaggagcgtcagcgcggtatcaccatcaacatcgcgcacgtggagtaccagaccgacaagcggcactacgcacacgtcgacgcccctggccacgccgactacatcaagaacatgatcaccggcgccgcgcagatggacggtgcgatcctggtggtcgccgccaccgacggcccgatgccccagacccgcgagcacgttctgctggcgcgtcaagtgggtgtgccctacatcctggtagcgctgaacaaggccgacgcagtggacgacgaggagctgctcgaactcgtcgagatggaggtccgcgagctgctggctgcccaggaattcgacgaggacgccccggttgtgcgggtctcggcgctcaaggcgctcgagggtgacgcgaagtgggttgcctctgtcgaggaactgatgaacgcggtcgacgagtcgattccggacccggtccgcgagaccgacaagccgttcctgatgccggtcgaggacgtcttcaccattaccggccgcggaaccgtggtcaccggacgtgtggagcgcggcgtgatcaacgtgaacgaggaagttgagatcgtcggcattcgcccatcgaccaccaagaccaccgtcaccggtgtggagatgttccgcaagctgctcgaccagggccaggcgggcgacaacgttggtttgctgctgcggggcgtcaagcgcgaggacgtcgagcgtggccaggttgtcaccaagcccggcaccaccacgccgcacaccgagttcgaaggccaggtctacatcctgtccaaggacgagggcggccggcacacgccgttcttcaacaactaccgtccgcagttctacttccgcaccaccgacgtgaccggtgtggtgacactgccggagggcaccgagatggtgatgcccggtgacaacaccaacatctcggtgaagttgatccagcccgtcgccatggacgaaggtctgcgtttcgcgatccgcgagggtggccgcaccgtgggcgccggccgggtcaccaagatcatcaagtag

SEQ ID NO: 11 - protein, 295 aa, Mycobacterium tuberculosis
MLFGMARIAIIGGGSIGEALLSGLLRAGRQVKDLVVAERMPDRANYLAQTYSVLVTSAADAVENATFVVVAVKPADVEPVIADLANATAAAENDSAEQVFVTVVAGITIAYFESKLPAGTPVVRAMPNAAALVGAGVTALAKGRFVTPQQLEEVSALFDAVGGVLTVPESQLDAVTAVSGSGPAYFFLLVEALVDAGVGVGLSRQVATDLAAQTMAGSAAMLLERMEQDQGGANGELMGLRVDLTASRLRAAVTSPGGTTAAALRELERGGFRMAVDAAVQAAKSRSEQLRITPE

SEQ ID NO: 12 - DNA, 888 bp, Mycobacterium tuberculosis
atgcttttcggcatggcaaggatcgcgattatcggcggcggcagcatcggtgaggcattgctgtcgggtctgctgcgggcgggccggcaggtcaaagacctggtagtggccgagcggatgcccgatcgcgccaactacctggcgcagacctattcggtgttggtgacgtcggcggccgacgcggtggagaacgcgacgttcgtcgtcgtcgcggtcaaaccagccgacgtcgagccggtgatcgcggatctggcgaacgcgactgcggcggccgaaaacgacagtgctgagcaggtgttcgtcaccgtggtagcgggcatcacgatcgcgtatttcgaatccaagctaccggctgggacgccagtggtgcgtgcgatgccgaacgcggcggcattggtgggagcgggggttacagcgctggccaaaggccgctttgtcaccccgcaacagcttgaggaggtctcggccttgttcgacgcggtcggcggcgtgctgaccgttccggaatcgcagttggacgcggtgaccgcggtgtccggctcgggtccggcctatttctttctgctggtcgaggccctggtggatgccggagtcggggtgggcttgagccgtcaggtggccaccgatctcgccgcgcagacaatggctggctcagcggcgatgctgctggagcggatggagcaagaccagggtggcgccaatggcgagctgatggggctgcgcgtggaccttaccgcatcacggctgcgcgccgcggttacctcgccgggcggtacgaccgccgctgcgctgcgggaactcgaacgcggcgggtttcggatggctgtcgacgcggcggttcaagccgccaaaagccgctctgagcagctcagaattacaccggaatga

SEQ ID NO: 13 - protein, 213 aa, Mycobacterium tuberculosis
MPRQAGRWSPTALRILGAAAELIALRGYSSTSTRDIAAAVGVEQPAIYKHFSAKRDILAALVRLAVEWPLELFGHITAMPVPAVVKLHRWLTESLDHLHASPYVLVSILITPDLHQESFVAERELVAEMERALVGLIETGQGEGDVRAMHPLSAARLVQALFDALALPEFAVSPDEIVEFAMTALLSDPDRLAEIRAAADALEIQTAPPDRGL

SEQ ID NO: 14 - DNA, 642 bp, Mycobacterium tuberculosis
atgccgaggcaggccggccgctggagcccaacggcactccgcatcctgggggccgctgccgagctcatcgcgctgcggggttacagctccacctcgacgcgagacattgccgcagccgtcggggtggagcagccggccatctacaaacacttctcggccaaacgcgatatccttgcggccctggtgcggctcgccgtggagtggccgcttgagctgttcggtcacatcaccgcgatgccggttccggcggtggtcaagctgcaccgctggctgacggaatccctggaccacctgcatgcttcgccctatgtgctggtttccatcctcatcaccccggacctgcatcaggaatccttcgtcgcggagcgggagctagtcgcggagatggaacgcgcgctggttggcctgatcgagaccggccaaggcgaaggtgatgtgcgcgcaatgcatccgttatcggctgccaggctggtgcaggcgttgtttgacgctctggcgctgccggaattcgcggtcagcccggacgagatcgtcgagttcgcgatgacggcgctgttgtcggaccctgatcggctagccgagatccgggcagcagcagatgctttggagatccagaccgcaccccccgaccgcgggctctag

SEQ ID NO: 15 - protein, 457 aa, Mycobacterium tuberculosis
MTGPGSPPLAWTELERLVAAGDVDTVIVAFTDMQGRLAGKRISGRHFVDDIATRGVECCSYLLAVDVDLNTVPGYAMASWDTGYGDMVMTPDLSTLRLIPWLPGTALVIADLVWADGSEVAVSPRSILRRQLDRLKARGLVADVATELEFIVFDQPYRQAWASGYRGLTPASDYNIDYAILASSRMEPLLRDIRLGMAGAGLRFEAVKGECNMGQQEIGFRYDEALVTCDNHAIYKNGAKEIADQHGKSLTFMAKYDEREGNSCHIHVSLRGTDGSAVFADSNGPHGMSSMFRSFVAGQLATLREFTLCYAPTINSYKRFADSSFAPTALAWGLDNRTCALRVVGHGQNIRVECRVPGGDVNQYLAVAALIAGGLYGIERGLQLPEPCVGNAYQGADVERLPVTLADAAVLFEDSALVREAFGEDVVAHYLNNARVELAAFNAAVTDWERIRGFERL

SEQ ID NO: 16 - DNA, 1374 bp, Mycobacterium tuberculosis
gtgaccggccccggttcgccgccgttggcgtggaccgagttggagcgactggtcgcggccggtgacgtcgacaccgtcatcgtcgcgttcaccgacatgcagggccggctggccggcaaacggatatcgggccggcatttcgtcgacgacatagccacccgcggcgtcgagtgctgcagttatctgctggccgtggacgtcgacctgaacacggtgcccggctatgcgatggccagttgggacaccggctacggcgatatggtgatgacgccggacttgtccactctgcggctgattccttggctaccgggaacggcgctggtgatcgccgacctggtctgggccgacggcagcgaggtcgccgtctcgccgcgcagcattctgcgccgtcagctcgatcggctcaaggcgcgcggactggtcgccgatgtggccaccgagctggagttcatcgtgttcgaccagccgtatcgccaggcatgggccagcgggtatcgcgggctgaccccggccagcgactacaacatcgactacgcgatattggcatcctcgcggatggagccgttgctgcgcgacatccggttgggtatggccggtgcgggtctgcgattcgaggcggtcaaaggcgaatgcaacatgggccagcaggagatcgggtttcgttacgacgaggcgctggtcacctgcgacaaccatgcgatctacaagaacggcgccaaggaaatcgccgaccagcacggcaagagcctaacgttcatggcgaaatacgatgaacgcgaaggtaatagctgtcacatccatgtctcgctgcgtggcacggatggctccgcggtgtttgccgacagtaacgggccgcacggcatgtcgtcgatgttccgcagcttcgtcgccggccagttggccacgttgcgcgaattcacgctgtgctatgcgccgaccattaactcctacaagcgatttgccgatagcagtttcgcgccgacggcgctggcttgggggctggacaatcgcacctgcgccctgcgggtggttggccacgggcaaaacatccgggtcgaatgccgggttcccggcggtgatgtcaaccagtacctggcggtggcggctctcattgctggagggttgtacggtatcgagcggggccttcagctgcccgagccctgtgtcggcaacgcctaccaaggcgccgatgtcgaacggctgccggttacgctggccgacgccgcggtgctgttcgaggattctgcgctggtgcgcgaggcgttcggcgaggatgttgtcgcgcactacctgaacaacgcgcgtgtggagctggcggcgttcaacgcggcggtcaccgattgggagaggatacgtggatttgagcgcctctag

SEQ ID NO: 17 - DNA, 18 bp, artificial
  primer sequence for amplificaiotn of Mycobacterium of M.tb complex
cgttcccgggccttgtac

SEQ ID NO: 18 - DNA, 21 bp, artificial
  primer sequence for amplificaiotn of Mycobacterium of M.tb complex
cgggtgttaccgactttcatg

SEQ ID NO: 19 - DNA, 21 bp, artificial
  primer sequence for amplification of Mycobacterium of M.tb complex
ggcaacaaacagctcgaagag

SEQ ID NO: 20 - DNA, 20 bp, artificial
  primer sequence for amplification of Mycobacterium of M.tb complex
catcaggtcgcggagtttct

SEQ ID NO: 21 - DNA, 18 bp, artificial
  primer sequence for amplification of Mycobacterium of M.tb complex
accaaggcctccgatgtg

SEQ ID NO: 22 - DNA, 17 bp, artificial
  primer sequence for amplification of Mycobacterium of M.tb complex
agacgtgcgccgtctca

SEQ ID NO: 23 - DNA, 21 bp, artificial
  primer sequence for amplification of Mycobacterium of M.tb complex
cgaatgactatcgccgatgaa

SEQ ID NO: 24 - DNA, 20 bp, artificial
  primer sequence for amplification of Mycobacterium of M.tb complex
gccatgtgcgtgaacaagat

SEQ ID NO: 25 - DNA, 20 bp, artificial
  primer sequence for amplification of Mycobacterium of M.tb complex
cacaatcgttcgtgttggag

SEQ ID NO: 26 - DNA, 20 bp, artificial
  primer sequence for amplification of Mycobacterium of M.tb complex
gtgcaccttgtttgggaagt

SEQ ID NO: 27 - DNA, 19 bp, artificial
  primer sequence for amplification of Mycobacterium of M.tb complex
aggttggtgtgatcggcta

SEQ ID NO: 28 - DNA, 20 bp, artificial
  primer sequence for amplification of Mycobacterium of M.tb complex
aggccctgctcttctacctt

SEQ ID NO: 29 - DNA, 18 bp, artificial
  primer sequence for amplification of Mycobacterium of M.tb complex
gtgtgccgtgtttggttg

SEQ ID NO: 30 - DNA, 19 bp, artificial
  primer sequence for amplification of Mycobacterium of M.tb complex
gatcgctttggcatacgac

SEQ ID NO: 31 - DNA, 19 bp, artificial
  primer sequence for amplification of Mycobacterium of M.tb complex
tggcgctgtcgtatgccaa

SEQ ID NO: 32 - DNA, 20 bp, artificial
  primer sequence for amplification of Mycobacterium of M.tb complex
cacaacaccgtttgctcacc

SEQ ID NO: 33 - DNA, 20 bp, artificial
  Molecualr beacon sequence for detection of Mycobacterium of M.tb complex
aagacgacgttcaaagacga

SEQ ID NO: 34 - DNA, 30 bp, artificial
  Molecular beacon sequence for detection of Mycobacterium of M.tb complex
ccgggaagacgacgttcaaagacgacccgg

SEQ ID NO: 35 - DNA, 20 bp, artificial
  primer sequence for amplification of Mycobacterium of M.tb complex
cgtgtttggttgcggtagag

SEQ ID NO: 36 - DNA, 20 bp, artificial
  primer sequence for amplification of Mycobacterium of M.tb complex
gtttgctcaccgaacaggtc

SEQ ID NO: 37 - DNA, 27 bp, artificial
  primer sequence for amplification of Mycobacterium of M.tb complex
ccgcacaacaccgtttgctcaccgaac

SEQ ID NO: 38 - DNA, 46 bp, artificial
  primer sequence for amplification of Mycobacterium of M.tb complex
aattctaatacgactcactatagggtcacaacaccgtttgctcacc

SEQ ID NO: 39 - DNA, 46 bp, artificial
  primer sequence for amplification of Mycobacterium of M.tb complex
aattctaatacgactcactatagggtgtttgctcaccgaacaggtc

SEQ ID NO: 40 - DNA, 53 bp, artificial
  primer sequence for amplification of Mycobacterium of M.tb complex
aattctaatacgactcactatagggtccgcacaacaccgtttgctcaccgaac

(19) United States
(12) Patent Application Publication
(10) Pub. No.: US 2012/0165246 A1
(43) Pub. Date: Jun. 28, 2012

(54) Method of Diagnosis of Infection by Mycobacteria and Reagents Therefor

(75) Inventors: Robyn Lindner, Five Dock (AU); Ian Garthwaite, Brookvale (AU)

(73) Assignee: TYRIAN DIAGNOSTICS LIMITED, Sydney (AU)

(21) Appl. No.: 13/202,949

(22) PCT Filed: Feb. 26, 2010
(86) PCT No.: PCT/AU2010/000231

§ 371 Date: Feb. 27, 2012

(30) Foreign Application Priority Data
Feb. 26, 2009 (AU) 2009900876

(51) Int. Cl. A61K 38/16; C12Q 1/70; A61P 31/06; C12Q 1/68

(52) U.S. Cl. 514/2.4; 435/6.15; 435/6.11; 435/6.12; 435/5

(57) ABSTRACT

The present invention provides a method of specifically detecting the presence of one or more Mycobacteria of the M. tuberculosis complex, said method comprising detecting ilvC nucleic acid of one or more Mycobacteria of the M. tuberculosis complex in a sample under conditions that do not detect ilvC nucleic acid of the M. avium complex. The invention also provides methods of diagnosis and treatment of tuberculosis in a subject employing the specific detection ilvC nucleic acid of one or more Mycobacteria of the M. tuberculosis complex.

### CROSS-REFERENCE TO RELATED APPLICATIONS

This application claims priority from Australian Patent Application No. 2009900876 filed Feb. 26, 2009 the contents of which are incorporated herein in their entirety.

### BACKGROUND OF THE INVENTION

1. Field of the Invention

The invention provides relates to the detection of one or more Mycobacteria of the M. tuberculosis complex, and to the diagnosis and prognosis of infection of an animal subject such as a human by Mycobacteria of the M. tuberculosis complex, and the diagnosis of conditions associated with such infections, especially tuberculosis. More particularly, the present invention relates to the expression in an infected subject of a Ketol-acid reductoisomerase (KARI) mRNA of M. tuberculosis and encoded protein (SEQ ID NO:1) thereof, and reagents for novel diagnostic and prognostic methods and for monitoring efficacy of therapy of infection and/or treatment of tuberculosis.

2. Description of the Related Art

Tuberculosis (TB) is a chronic, infectious disease that is generally caused by infection with Mycobacterium tuberculosis or by one or more organisms of the Mycobacterium tuberculosis complex. As used herein, the term “Mycobacterium tuberculosis complex” means one or more organisms selected from the group consisting of M. tuberculosis, M. bovis, M. africanum, M. canetti and M. microti. The skilled artisan is also aware that the M. tuberculosis complex is distinct from the so-called M. avium complex including M. avium and M. intracellulaire which are causative agents of the unrelated disease known as paratuberculosis e.g., in agricultural animals.

Tuberculosis (TB) is a major disease in developing countries, as well as an increasing problem in developed areas of the world, with about eight million new cases and three million deaths each year. Although the infection may be asymptomatic for a considerable period of time, the disease is most commonly manifested as an acute inflammation of the lungs, resulting in fever and a productive cough. If left untreated, M. tuberculosis infection may progress beyond the primary infection site in the lungs to any organ in the body and generally results in serious complications and death.

The problem of the rapidly growing global incidence of TB has been often described by many workers in the health care industry and is well known to skilled artisans in that field. As treatment for M. tuberculosis infection requires many months of therapy with multiple drugs, such an extensive course of treatment can result in poor compliance and the emergence of multidrug resistant (MDR) and extensively drug resistant (XDR) TB.

The problem is worsened by HIV infection. The incidence of tuberculosis is especially common in late-staging AIDS patients, a majority of whom suffer from it. In fact, HIV infection is a most important risk factor for the development of active tuberculosis in purified protein derivative (PPD)-tuberculin-positive subjects, and the risk of acquisition of tuberculosis infection in HIV-infected immunosuppressed individuals may be markedly enhanced compared to those individuals that are not HIV-infected. It is also likely that co-infections with HIV-1, and M. tuberculosis mediate a shortened HIV symptom-free period and shortened survival time in subjects, possibly by triggering increased viral replication and virus load that results in depletion of CD4+ T-cells and immune deficiency or immune suppression (Corbett et al 2003; Ho, Mem. Inst. Oswaldo Cruz, 91, 385-387, 1996).

Despite the enormous global burden of tuberculosis, case detection also continues to be a problem. It is estimated that approximately half of all patients with TB are still not diagnosed and appropriately treated (WHO Report 2007, WHO/HTM/TB/2007.376, Geneva Switzerland). It is evident from these data that early accurate diagnosis of acute infection, as well as, diagnosis of latent infection is necessary to control tuberculosis.

Conventional tuberculosis diagnosis continues to rely on sputum smear microscopy for the detection of acid-fast bacilli, culture, tuberculin skin test, and chest radiography, all of which are known to have several limitations. In the detection of active tuberculosis infection, microscopy is rapid and inexpensive, but has low sensitivity requiring the visualization of bacilli in sputum smear samples. Culture is more sensitive, but results can take several weeks and has the incidence of 10-20% of false-positives. Despite these tests being in use for nearly a century, they have been insufficient to control tuberculosis particularly in developing countries and where HIV is epidemic.

The development of diagnostic tests for tuberculosis have been hampered by numerous difficulties. Until the present invention, the identification of biomarkers amenable for use in detecting infection by one or more bacteria of the M. tuberculosis complex has been problematic. This is because it was not clear which M. tuberculosis proteins were the most highly expressed of M. tuberculosis in any particular environment in vivo. Furthermore, expression in different environments has complicated the identification of infectivity markers and markers that would distinguish active infection compared to latent infection. Laboratory systems and the use of laboratory strains do not meet this deficit. Moreover, it has been difficult to identify such a marker that would be useful for the detection of the other mycobacterium of the M. tuberculosis complex. Even with the identification of putative markers, diagnosis of TB is further complicated by the lack of sensitivity of current tests, and reproducibility across a large cohort of clinical samples. Molecular tests relying on the amplification of DNA and rRNA target sequences have not been suitable as tests for acute infection, or as infectivity markers. Additionally, molecular tests based on detection of mRNA meet with the same obstacles as tests based on protein detection, with the further complication of message stability particularly during storage of clinical samples. Accordingly, there is a need in the art for reagents that provide the required sensitivity and selectivity for detecting one or more bacteria of the M. tuberculosis complex in clinical samples over mycobacterial proteins in general

Progress in identifying a suitable biomarker that is expressed at high levels has met several obstacles in the art. Whilst the sequencing of the Mycobacterium tuberculosis genome has facilitated an enormous research effort to identify M. tuberculosis proteins and mRNA that theoretically may be expressed by the organism sequence data alone are insufficient to conclude that any particular protein or mRNA is expressed in vivo by the organism, let alone during infection of a human or other animal subject. The mere elucidation of open reading frames in the genome of M. tuberculosis is not indicative that any particular protein encoded is actually expressed by the bacterium, and may be used as an antigen or mRNA based diagnostic. For example, to conclude that a particular protein or mRNA encoding a protein of M. tuberculosis has efficacy as a diagnostic reagent in an antigen-based test, or a nucleic acid amplification test (NAAT), protein or mRNA must be present and accessible in the sample, which reflects the protein expression during infectious cycle of the bacterium. Moreover, the known instability of mRNA during storage of clinical samples makes diagnostic testing based on mRNA detection a particularly challenging feat, in terms of actual detection, and reproducibility across large cohorts of clinical samples.

The ability to grow M. tuberculosis in culture has provided a convenient model to identify expressed tuberculosis proteins in vitro. However, the culture environment is markedly different to the environment of a human macrophage, lung, or extrapulmonary site where M. tuberculosis is found in vivo. Furthermore, M. tuberculosis is both an intercellular pathogen and an intracellular pathogen. Recent evidence indicates that the protein expression profile of intracellular parasites varies markedly depending on environmental cues, such that the expression profile of the organism in vitro may not accurately reflect the expression profile of the organism in situ. This would similarly apply to M. tuberculosis during its intracellular state.

Infection with M. tuberculosis bacilli, or reactivation of a latent infection, induces a host response comprising the recruitment of monocytes and macrophages to the site of infection. As more immune cells accumulate a nodule of granulomata forms comprising immune cells and host tissue that have been destroyed by the cytotoxic products of macrophages. As the disease progresses, macrophage enzymes cause the hydrolysis of protein, lipid and nucleic acids resulting in liquefaction of surrounding tissue and granuloma formation. Eventually the lesion ruptures and the bacilli are released into the surrounding lung, blood or lymph system.

During this infection cycle, the bacilli are exposed to four distinct host environments, being alveoli macrophage, caseous granuloma, extracellular lung and extrapulmonary sites, such as, for example the kidneys or peritoneal cavities, lymph, bone, or spine.

It is thought that bacilli can replicate to varying degrees in all these environments, however, little is known about the environmental conditions at each site. All four host environments are distinct, suggesting that the expression profile of M. tuberculosis in each environment will be different.

Accordingly, the identification of M. tuberculosis proteins from logarithmic phase cultures does not necessarily suggest which proteins or mRNA are expressed in each environment in vivo. Similarly, the identification of M. tuberculosis proteins in a macrophage grown in vitro will not necessarily emulate the protein or mRNA expression profile of M. tuberculosis in caseous granuloma, highly aerated lung, or at an extrapulmonary site having a low oxygen content.

Furthermore, M. tuberculosis infection within the host can be seen as a dynamic event where the host immune system is continually trying to encapsulate and destroy bacilli through destruction of infected macrophages. Consequently, the M. tuberculosis bacilli progress through cycles of intracellular growth, destruction (where both intracellular and secreted bacterial proteins are exposed and destroyed), and rapid extracellular multiplication. Host and pathogen interaction is a result of many factors, which can not be replicated in vitro.

Although systems for the amplification of DNA, and rRNA target sequences that are specific for members of the M. tuberculosis complex have been described, such tests have not been suitable for monitoring the response of patients to treatment due to the persistence of the nucleic acid targets. This is because the stability of bacterial DNA and rRNA can mean that amplifiable DNA and rRNA targets can persist in a patient. The persistence of these nucleic acid species is thought to reflect shedding of dead or dormant bacilli from pulmonary lesions, and as a consequence are not a good indicator of acute infection. Such nucleic acid based tests have also not been successful or recommended by the WHO as a stand-alone test, without smear or culture confirmation.

Several nucleic acid amplification tests (NAATs) have emerged based on the identification of M. tuberculosis genes and hypothetical coding sequences. A recent meta-analysis of all available NAATs based on DNA, rRNA and mRNA targets indicates that actual performance of available tests has been less than optimal (Ling et al PLoS ONE 3 (2): e1536, February 2008). Sensitivity and specificity estimates of NAATs in respiratory specimens are highly variable, with sensitivity being lower and more inconsistent than specificity and summary measures of diagnostic accuracy are not considered clinically meaningful. Meta-analysis studies have confirmed that NAATs that are currently available cannot be recommended to replace conventional tests for diagnosing pulmonary TB, nor have they demonstrated usefulness for monitoring treatment progress, as NAAT are still problematic for the detection of non-viable bacteria and give false-positive results.

There clearly remains a need for improvements in diagnostic accuracy, particularly sensitivity, of diagnostic and prognostic reagents, that provide cost-effective and rapid determination of infection by M. tuberculosis and/or disease conditions associated therewith.

Conventional techniques of molecular biology, microbiology, proteomics, virology, recombining DNA technology, peptide synthesis in solution, solid phase peptide synthesis, and immunology described, for example, in the following texts:
1. Sambrook, Fritsch & Maniatis, Molecular Cloning: A Laboratory Manual, Cold Spring Harbor Laboratories, New York, Second Edition (1989), whole of Vols I, II, and III;
2. DNA Cloning: A Practical Approach, Vols. I and II (D. N. Glover, ed., 1985), IRL Press, Oxford, whole of text;
3. Oligonucleotide Synthesis: A Practical Approach (M. J. Gait, ed., 1984) IRL Press, Oxford, whole of text, and particularly the papers therein by Gait, pp 1-22; Atkinson et al., pp 35-81; Sproat et al., pp 83-115; and Wu et al., pp 135-151;
4. Nucleic Acid Hybridization: A Practical Approach (B. D. Hames & S. J. Higgins, eds., 1985) IRL Press, Oxford, whole of text;
5. Immobilized Cells and Enzymes: A Practical Approach (1986) IRL Press, Oxford, whole of text;
6. Perbal, B., A Practical Guide to Molecular Cloning (1984);
7. Methods In Enzymology (S. Colowick and N. Kaplan, eds., Academic Press, Inc.), whole of series;
8. J. F. Ramalho Ortigão, “The Chemistry of Peptide Synthesis” In: Knowledge database of Access to Virtual Laboratory website (Interactiva, Germany);
9. Sakakibara, D., Teichman, J., Lien, E. Land Fenichel, R. L. (1976). Biochem. Biophys. Res. Commun. 73 336-342
10. Merrifield, R. B. (1963). J. Am. Chem. Soc. 85, 2149-2154.
11. Barany, G. and Merrifield, R. B. (1979) in The Peptides (Gross, E. and Meienhofer, J. eds.), vol. 2, pp. 1-284, Academic Press, New York.
12. Wünsch, E., ed. (1974) Synthese von Peptiden in Houben-Weyls Metoden der Organischen Chemie (Müller, E., ed.), vol. 15, 4th edn., Parts 1 and 2, Thieme, Stuttgart.
13. Bodanszky, M. (1984) Principles of Peptide Synthesis, Springer-Verlag, Heidelberg.
14. Bodanszky, M. & Bodanszky, A. (1984) The Practice of Peptide Synthesis, Springer-Verlag, Heidelberg.
15. Bodanszky, M. (1985) Int. J. Peptide Protein Res. 25, 449-474.
16. Handbook of Diagnostic testal Immunology, Vols. I-IV (D. M. Weir and C. C. Blackwell, eds., 1986, Blackwell Scientific Publications).
17. Wilkins M. R., Williams K. L., Appel R. D. and Hochstrasser (Eds) 1997 Proteome Research: New Frontiers in Functional Genomics Springer, Berlin.

### SUMMARY OF INVENTION

In work leading up to the present invention, the inventors sought to identify a biomarker of TB infection by one or more of the mycobacterium of the M. tuberculosis complex that is expressed, i.e., at the protein level and/or RNA level, preferably at levels correlated with bacterial load, and in clinical samples and clinical strains; and/or to provide reagents for the detection of specific mycobacterium of the M. tuberculosis complex as opposed to the generic detection of mycobacteria; and/or to develop a test having improved sensitivity and/or selectivity of detection in clinical samples, including stored samples, compared with conventional tests; and/or to develop a test having the ability to reproducibly detect M. tuberculosis complex organisms across a panel of clinical samples including stored samples. The present invention is based in part on the discovery of a range of proteins expressed by M. tuberculosis complex organism(s) in a range of in vivo environments, to thereby identify highly expressed and/or highly immunogenic proteins of M. tuberculosis and other organism(s) of the M. tuberculosis complex. Such proteins have been described in several patent applications, all of which are incorporated herein by reference (International Publication Numbers: WO 2006/01792, WO 2007/087679, WO 2007/131291, WO 2007/140545, WO 2007/131293, WO 2007/131292, WO 2006/000045). A proteomics approach was used to identify M. tuberculosis complex proteins expressed in vivo and present in the body fluids of a cohort of diseased patients, including sputum, pleural fluid, plasma and serum. An M. tuberculosis complex protein was identified in vivo by 2-dimensional electrophoresis of immunoglobulin-containing samples, in particular IgG, obtained previously from a cohort of patients diagnosed with tuberculosis e.g., patients infected with M. tuberculosis or other organism of the M. tuberculosis complex. A peptide fragment was identified, and the amino acid sequences of peptide fragments were determined by mass spectrometry of tryptic fragments, and shown to align to the amino acid sequence of the Ketol-Acid Reducto Isomerase (KARI) protein (SEQ ID NO: 1). In particular, a matched peptide aligned to a region of the KARI protein sequence. Nucleic acid encoding the KARI protein of SEQ ID NO: 1, is encoded by the ilvC gene of M. tuberculosis or a homolog.

The inventors used these findings to develop a nucleic acid amplification test using several previously identified tuberculosis markers found to be present as described supra in clinical samples. In a comparison quantitative real-time RT-PCR assay for detection of expressed tuberculosis biomarkers, the inventors found that an ilvC transcript was the most reliable, with the highest number of transcript copies detectable in clinical samples. Transcript levels of ilvC also correlate with severity of infection in patients.

These findings have provided the means for producing hybridization-based diagnostic tests e.g., FISH, and nucleic acid amplification (NAA) based diagnostic tests including NASBA-based platforms such as PCR-based tests and isothermal amplification methods that do not require thermal cycling, and including amplification methods performed in real time e.g., real time PCR and real-time RT-PCR and real-time isothermal methods and real time NASBA, and including such tests performed with the aid of labelled reporters that bind to the primers e.g., SYBR-green I and/or II based, or with probes that bind to amplicons e.g., scorpion probes, TaqMan probes and molecular beacons. These tests are useful for the specific detection of infection by M. tuberculosis complex organisms, and for the diagnosis of tuberculosis or monitoring of infection or therapy or for prognosis of infection or a disease state associated therewith e.g., by virtue of the detection of M. tuberculosis and/or other M. tuberculosis complex organisms in a subject. Such NAA-based diagnostics and prognostics are based on the highly specific and sensitive detection of ilvC transcripts (ilvC-NAA) in clinical samples. For example, the present inventors are able to detect at least about 10³ ilvC-encoding RNA copies in a reaction. It will also be apparent to the skilled person that such diagnostic and prognostic tests may be used in conjunction with therapeutic treatments for tuberculosis or an infection associated therewith e.g., to determine efficacy of therapeutic intervention.

Accordingly, the present invention provides a method of specifically detecting the presence of one or more Mycobacteria of the M. tuberculosis complex, said method comprising detecting nucleic acid of one or more Mycobacteria of the M. tuberculosis complex in a biological sample using a detection means as described herein. For example, the method is performed using the detection means under conditions that do not detect nucleic acid of the M. avium complex.

In one example, the present invention provides a method of specifically detecting the presence of one or more Mycobacteria of the M. tuberculosis complex, said method comprising detecting ilvC nucleic acid of one or more Mycobacteria of the M. tuberculosis complex in a sample under conditions that do not detect ilvC nucleic acid of the M. avium complex.

In one example, detection is performed by standard nucleic acid hybridization or a variant thereof e.g., FISH or any other method that does not comprise a nucleic acid amplification. This is in keeping with the spirit of the invention in showing that ilvC may be employed conveniently under suitable conditions as a biomarker for the specific detection of Mycobacteria of the M. tuberculosis complex, and at acceptable levels of sensitivity.

Accordingly, the detected ilvC nucleic acid may comprise a sequence of M. tuberculosis ilvC DNA or RNA. The M. tuberculosis complex organism may be selected individually or collectively from M. tuberculosis, M. bovis, M. africanum, M. canetti and M. microti or a combination thereof. The M. tuberculosis complex organism is selected from M. tuberculosis and M. bovis or a combination thereof. The M. tuberculosis complex organism may be M. tuberculosis or a clinical strain or clinical isolate of M. tuberculosis. The M. tuberculosis complex organism may be M. bovis. It will be known to the skilled artisan that a Mycobacterium of the M. avium complex may be M. avium or M. intracellulaire.

In a particularly preferred example, the inventive method for detecting ilvC nucleic acid of one or more Mycobacteria of the M. tuberculosis complex comprises performing an amplification reaction e.g., any one of several NASBA-based assays such as an isothermal amplification or other amplification not requiring thermal cycling, or an amplification reaction requiring thermal cycling such as a polymerase chain reaction (PCR) e.g., a standard PCR or reverse-transcriptase mediated PCR (RT-PCR). The examples provided herein clearly support methods requiring thermal cycling as well as methods that do not employ thermal cycling. Assording the term “amplification” in the present context should be construed to encompass all amplification methods without reference to thermal cycling, unless specifically indicated otherwise. Amplification assay formats not specifically stated herein are not to be excluded.

For example, detecting ilvC nucleic acid of one or more Mycobacteria of the M. tuberculosis complex may comprise amplifying one or more ilvC nucleic acids to thereby produce amplified ilvC nucleic acid and detecting the amplified nucleic acid, wherein the detection of amplified ilvC nucleic acid is indicative of the presence of one or more of said Mycobacteria of the M. tuberculosis complex in the sample. In accordance with this example, detecting ilvC nucleic acid of one or more Mycobacteria of the M. tuberculosis complex without detecting ilvC nucleic acid of the M. avium complex comprises performing an amplification reaction e.g., an amplification reaction requiring thermal cycling or amplification reaction that does not require thermal cycling, to thereby detect ilvC nucleic acid comprising a sequence of at least 12 or 15 or 20 or 40 or 50 contiguous nucleotides in length of SEQ ID NO: 2 or a homologous sequence thereto from one or more Mycobacteria of the M. tuberculosis complex. As used herein, the term “detect ilvC nucleic acid comprising a sequence of at least 20 or 40 or 50 contiguous nucleotides in length” refers to the minimum region in the ilvC nucleic acid of the sample that is hybridized specifically to a primer or probe. Alternatively, or in addition, detecting ilvC nucleic acid of one or more Mycobacteria of the M. tuberculosis complex without detecting ilvC nucleic acid of the M. avium complex comprises performing an amplification reaction e.g., an amplification reaction requiring thermal cycling or amplification reaction that does not require thermal cycling, to thereby produce an amplicon of at least 50 or 60 or 70 or 80 or 90 or 100 or 110 or 120 or 130 or 140 or 150 or 160 or 170 or 180 or 190 or 200 contiguous nucleotides in length of SEQ ID NO: 2 or a homologous sequence thereto from one or more Mycobacteria of the M. tuberculosis complex. The skilled artisan is aware that the term “amplicon” refers to amplified nucleic acid.

Alternatively, or in addition, detecting ilvC nucleic acid of one or more Mycobacteria of the M. tuberculosis complex without detecting ilvC nucleic acid of the M. avium complex comprises performing an amplification reaction e.g., an amplification reaction requiring thermal cycling or amplification reaction that does not require thermal cycling, using one or a plurality of primers that each comprise a sequence of at least about 18 contiguous nucleotides of SEQ ID NO: 2 from a region of SEQ ID NO: 2 selected from the group consisting of:
(i) position 420 to position 600 of SEQ ID NO: 2 or a sequence complementary to SEQ ID NO: 2 from position 420 to position 600 thereof;
(ii) position 40 to position 180 of SEQ ID NO: 2 or a sequence complementary to SEQ ID NO: 2 from position 40 to position 180 thereof;
(iii) position 880 to position 1000 of SEQ ID NO: 2 or a sequence complementary to SEQ ID NO: 2 from position 880 to position 1000 thereof.

For example, a forward primer is selected from the group consisting of SEQ ID NOs: 19, 27, 29, 31, and 35. In another example, a reverse primer is selected from the group consisting of SEQ ID NOs: 20, 28, 30, 32, 36, 37, 38, 39 and 40.

Alternatively, or in addition, detecting ilvC nucleic acid of one or more Mycobacteria of the M. tuberculosis complex without detecting ilvC nucleic acid of the M. avium complex comprises performing an amplification reaction e.g., an amplification reaction requiring thermal cycling or amplification reaction that does not require thermal cycling, for less than about 30 amplification cycles e.g., for about 12 amplification cycles to about 27 amplification cycles or for about 14 amplification cycles to about 20 amplification cycles.

Alternatively, or in addition, detecting ilvC nucleic acid of one or more Mycobacteria of the M. tuberculosis complex without detecting ilvC nucleic acid of the M. avium complex comprises performing an amplification reaction e.g., an amplification reaction requiring thermal cycling or amplification reaction that does not require thermal cycling, on less than about 2 ng/ml input nucleic acid. By “input nucleic acid” is meant nucleic acid that is included in a PCR reaction, and does not necessarily mean ilvC nucleic acid.

The ilvC nucleic acid of one or more Mycobacteria of the M. tuberculosis complex is detected in any one of a number of amplification platforms, optionally employing a quantitation means. One exemplary quantitation means comprises performing an amplification reaction e.g., an amplification reaction requiring thermal cycling or amplification reaction that does not require thermal cycling, using one or a plurality of amplification primers and a labelled probe comprising a sequence capable of hybridizing to a nucleic acid product of the amplification primers to thereby produce a detectable signal. In another example, an amplification reaction e.g., an amplification reaction requiring thermal cycling or amplification reaction that does not require thermal cycling, is performed using one or a plurality of amplification primers and a labelled probe capable of binding to at least one of the amplification primers.

The present invention is useful for detecting Mycobacteria of the M. tuberculosis complex in any one of a number of different sample types. For example the sample may comprise cultured cells of any M. tuberculosis complex organism. Alternatively, the sample may comprise sputum and/or broncho alveolar lavage (BAL) and/or a lymph node biopsy and/or blood or a fraction thereof e.g., serum, plasma, a fraction of serum or a fraction of plasma. Alternatively, the sample may comprise urine. Other samples known to comprise Mycobacteria of the M. tuberculosis complex are not to be excluded.

The method of the present invention according to any example hereof is capable of detecting at least about 10⁴ copies of ilvC nucleic acid in less than about 60 minutes, preferably at least about 10³ copies of ilvC of ilvC nucleic acid in less than about 60 minutes. Alternatively or in addition, the method of the present invention according to any example hereof is capable of detecting at least about 10⁴ CFU/ml Mycobacteria of the M. tuberculosis complex.

The method of the present invention is also amenable to multi-analyte formats e.g., a multiplex amplification reaction e.g., an amplification reaction requiring thermal cycling or amplification reaction that does not require thermal cycling such as multiplex-NASBA or multiplex-PCR, wherein the specific detection of ilvC nucleic acid of Mycobacteria of the M. tuberculosis complex is supplemented by the detection of one or more nucleic acids of one or more Mycobacteria of the M. tuberculosis complex other than ilvC nucleic acid. For example, a nucleic acid other than ilvC nucleic acid may be 16s rRNA of Mycobacteria of the M. tuberculosis complex. Alternatively, or in addition, one or more nucleic acids other than ilvC nucleic acid may encode a protein selected from the group consisting of BSX, S9, Rv1265, EF-Tu, P5CR, TetR-like protein and glutamine synthetase, or nucleic acid complementary thereto e.g., to thereby detect at least 20 or 30 or 40 or 50 or 60 or 70 or 80 or 90 or 100 or more contiguous nucleotides in length of a sequence selected from the group consisting of SEQ ID NOs: 4, 6, 8, 10, 12, 14 and 16, or a sequence complementary thereto. The invention may also produce one or more amplicons each of at least 50 or 60 or 70 or 80 or 90 or 100 contiguous nucleotides in length derived from the one or more nucleic acids other than ilvC nucleic acid when said nucleic acid is present in the sample. Formats wherein combinations of these nucleic acids are detected are also encompassed by the invention.

The method of the present invention is also amenable to multi-platform formats e.g., a combination of NAA-based and antigen-based platforms, wherein the specific detection of ilvC nucleic acid of Mycobacteria of the M. tuberculosis complex is supplemented by the detection of one or more proteins of one or more Mycobacteria of the M. tuberculosis complex e.g., KARI protein encoded by ilvC nucleic acid and/or BSX and/or S9 and/or Rv1265 and/or EF-Tu and/or P5CR and/or TetR-like protein and/or glutamine synthetase. In this assay format, it is particularly preferred to utilize an antigen-based assay to detect the protein as described according to any example hereof.

The present invention also provides a method of detecting the presence of one or more Mycobacteria of the M. tuberculosis complex in a biological sample, said method comprising:

(i) providing a biological sample from the subject;
(ii) isolating nucleic acid from said sample;
(iii) amplifying one or more ilvC nucleic acids in an in vitro nucleic acid amplification assay to produce amplified nucleic acid; and
(iv) detecting the amplified nucleic acid,
wherein the positive detection of the amplified nucleic acid is indicative of the presence of one or more of said Mycobacteria of the M. tuberculosis complex in said sample.

In another example, the present invention also provides a method of detecting the presence of one or more Mycobacteria of the M. tuberculosis complex in a biological sample, said method comprising amplifying one or more ilvC nucleic acids in the presence of amplification primers and a probe e.g., a molecular beacon or TaqMan probe or Scorpion probe, to thereby produce a detectable signal when the probe hybridizes to ilvC nucleic acid, wherein the detectable signal is indicative of the presence of one or more of said Mycobacteria of the M. tuberculosis complex in said sample.

One or more NAA based assays described according to any embodiment herein for detecting ilvC nucleic acid(s) (ilvC-NAA) are useful for the early diagnosis of infection or disease and provide a means to detect active or past infection by mycobacteria of the M. tuberculosis complex by detecting the presence of ilvC nucleic acids in a variety of clinical samples.

In another example, the present invention provides a process for diagnosing an infection by one or more Mycobacteria of the M. tuberculosis complex in a subject, said process comprising performing a method according to any example hereof on a biological sample from a subject to thereby detect ilvC nucleic acid of one or more Mycobacteria of the M. tuberculosis complex in a sample under conditions that do not detect ilvC nucleic acid of the M. avium complex, wherein the detection of one or more ilvC nucleic acids of one or more Mycobacteria of the M. tuberculosis complex is indicative of infection. This process of the invention is particularly suited to diagnosing an active infection or a latent infection.

In another example, the present invention provides a process for diagnosing tuberculosis in a subject, said process comprising performing a method according to any example hereof on a biological sample from a subject to thereby detect ilvC nucleic acid of one or more Mycobacteria of the M. tuberculosis complex in a sample under conditions that do not detect ilvC nucleic acid of the M. avium complex wherein the detection of one or more ilvC nucleic acids of one or more Mycobacteria of the M. tuberculosis complex is indicative of tuberculosis. This process of the invention is particularly suited to diagnosing pulmonary tuberculosis or extrapulmonary tuberculosis. This process of the invention is also particularly useful in conjunction with other clinical tests for tuberculosis or conditions associated with tuberculosis or M. tuberculosis infection e.g., a diagnosis of one or more clinical symptoms of tuberculosis in a subject and/or a diagnosis of one or more clinical symptoms of immune suppression in a subject and/or a diagnosis of HIV infection in a subject.

In another example, the present invention provides a method of diagnosing tuberculosis or an infection by one or more Mycobacteria of the M. tuberculosis complex in a subject comprising detecting one or more ilvC nucleic acids of one or more Mycobacteria of the M. tuberculosis complex present in a biological sample from said subject, the presence of said ilvC nucleic acid in the sample being indicative of infection.

Accordingly, a further example of the present invention provides a method of diagnosing tuberculosis or an infection by one or more Mycobacteria of the M. tuberculosis complex in a subject comprising detecting one or more ilvC nucleic acids of one or more Mycobacteria of the M. tuberculosis complex present in a biological sample from said subject by performing polymerase chain reaction in the presence of a probe e.g., a molecular beacon or TaqMan probe or Scorpion probe, wherein the presence of said ilvC nucleic acid in the sample is detected by fluorescence of the probe and said fluorescence being indicative of infection.

In another example, the present invention provides a process for treating tuberculosis or infection by one or more mycobacteria of the M. tuberculosis complex comprising:
(i) performing a method according to any example hereof on a sample from a subject to thereby detect one or more mycobacteria of the M. tuberculosis complex in the sample; and
(ii) administering a therapeutically effective amount of a pharmaceutical composition to the subject to thereby reduce the number of pathogenic bacilli in the lung, blood or lymph system of the subject.

The present invention also provides a method of treatment of tuberculosis or infection by one or more mycobacteria of the M. tuberculosis complex comprising:
(i) performing a diagnostic method according to any embodiment described herein thereby detecting the presence of one or more mycobacteria of the M. tuberculosis complex in a biological sample from a subject; and
(ii) administering a therapeutically effective amount of a pharmaceutical composition to reduce the number of pathogenic bacilli in the lung, blood or lymph system of the subject.

The present invention also provides a method of treatment of tuberculosis or infection by one or more mycobacteria of the M. tuberculosis complex comprising:
(i) performing a diagnostic method according to any embodiment described herein thereby detecting the presence of one or more mycobacteria of the M. tuberculosis complex in a biological sample from a subject being treated with a first pharmaceutical composition; and
(ii) administering a therapeutically effective amount of a second pharmaceutical composition to reduce the number of pathogenic bacilli in the lung, blood or lymph system of the subject.

The present invention also provides a method of treatment of tuberculosis in a subject comprising performing a diagnostic method or prognostic method as described herein. In one embodiment, the present invention provides a method of prophylaxis comprising:
(i) detecting the presence of one or more mycobacteria of the M. tuberculosis complex infection in a biological sample from a subject; and
(ii) administering a therapeutically effective amount of a pharmaceutical composition to reduce the number of pathogenic bacilli in the lung, blood or lymph system of the subject.

The present invention also provides a kit for detecting one or more mycobacteria of the M. tuberculosis complex in a biological sample, said kit comprising:
(i) one or more primers for NAA-based detection; and
(ii) reagents suitable for amplification of a nucleic acid using the method of the invention (e.g., a buffer and/or one or more deoxynucleotides and/or a polymerase and/or control primers) and for quantitation of the amplified products.

Optionally, the kit is packaged with instructions for use.

In another example, the invention provides a kit further comprising reagents suitable for antigen-based detection, said kit further comprising:
(i) one or more isolated antibodies or immune reactive fragments thereof that bind specifically to the isolated or recombinant immunogenic KARI protein of one or more mycobacteria of the M. tuberculosis complex or an immunogenic KARI peptide or immunogenic KARI fragment or epitope thereof according to any embodiment described herein or to a combination or mixture of said peptides or epitopes or fragments or to a fusion protein or protein aggregate comprising said immunogenic KARI protein, peptide, fragment or epitope; and
(ii) means for detecting the formation of an antigen-antibody complex.

Optionally, the kit is packaged with instructions for use.

In another example, the invention provides a kit further comprising reagents suitable for antibody-based detection, said kit further comprising:
(i) isolated or recombinant immunogenic KARI protein of one or more mycobacteria of the M. tuberculosis complex or an immunogenic KARI peptide or immunogenic KARI fragment or epitope thereof according to any embodiment described herein or a combination or mixture of said peptides or epitopes or fragments; and
(ii) means for detecting the formation of an antigen-antibody complex.

Optionally, the kit is packaged with instructions for use.

### DEFINITIONS

This specification contains nucleotide and amino acid sequence information prepared using PatentIn Version 3.3. Each nucleotide sequence is identified in the sequence listing by the numeric indicator <210> followed by the sequence identifier (e.g. <210>1, <210>2, <210>3, etc). The length and type of sequence (DNA, protein (PRT), etc), and source organism for each nucleotide sequence, are indicated by information provided in the numeric indicator fields <211>, <212> and <213>, respectively. Nucleotide sequences referred to in the specification are defined by the term “SEQ ID NO:”, followed by the sequence identifier (eg. SEQ ID NO: 1 refers to the sequence in the sequence listing designated as <400>1).

The designation of nucleotide residues referred to herein are those recommended by the IUPAC-IUB Biochemical Nomenclature Commission, wherein A represents Adenine, C represents Cytosine, G represents Guanine, T represents thymine, Y represents a pyrimidine residue, R represents a purine residue, M represents Adenine or Cytosine, K represents Guanine or Thymine, S represents Guanine or Cytosine, W represents Adenine or Thymine, H represents a nucleotide other than Guanine, B represents a nucleotide other than Adenine, V represents a nucleotide other than Thymine, D represents a nucleotide other than Cytosine and N represents any nucleotide residue.

As used herein, the term “Ketol-acid reductoisomerase” or “KARI” will be taken to mean M. tuberculosis protein composition comprising or having at least about 80% identity to SEQ ID NO: 1 or substantially the same sequence as set forth in SEQ ID NO: 1 of the present application and/or comprising or having a sequence that is at least about 80% identical to the sequence encoded by an ilvC gene of a Mycobacterium tuberculosis, said composition being suitable for the purposes of producing immunogenic peptides or preparing antibodies that cross react with one or more Mycobacteria of the M. tuberculosis complex or clinical matrix from subjects infected with said one or more Mycobacteria and not requiring any other functionality e.g., a role in protein translation. Until the present invention, the M. tuberculosis protein set forth in SEQ ID NO: 1 was not shown to be expressed in vivo, or to be immunogenic or immunologically non-cross-reactive with other organisms, and information in relation to the KARI protein was derived from a bioinformatic analysis of open reading frames in the M. tuberculosis genome that encodes a polypeptide of SEQ ID NO: 1.

As used herein the term “derived from” shall be taken to indicate that a specified integer may be obtained from a particular source albeit not necessarily directly from that source.

Unless the context requires otherwise or specifically stated to the contrary, integers, steps, or elements of the invention recited herein as singular integers, steps or elements clearly encompass both singular and plural forms of the recited integers, steps or elements.

The embodiments of the invention described herein with respect to any single embodiment and, in particular, with respect to any protein or a use thereof in the diagnosis, prognosis or therapy of M. tuberculosis shall be taken to apply mutatis mutandis to any other embodiment of the invention described herein.

The diagnostic embodiments described here for individual subjects clearly apply mutatis mutandis to the epidemiology of a population, racial group or sub-group or to the diagnosis or prognosis of individuals having a particular MHC restriction. All such variations of the invention are readily derived by the skilled artisan based upon the subject matter described herein.

A reference herein to the detection or identification of M. tuberculosis and/or a reference to the diagnosis, prognosis or monitoring of tuberculosis or infection by M. tuberculosis clearly extends to the detection of any one or more organisms of the M. tuberculosis complex but not to the diagnosis of paratuberculosis and/or one or more organisms of the M. avium complex, unless the context requires otherwise. For example, as described herein the invention encompasses the use of antibodies that cross-react with M. tuberculosis KARI and fragments and one or more of M. avium and M. intracellulaire as a generic screen for mycobacteria, coupled to the use of one or more surrogate assays for detecting tuberculosis and/or for detecting one or more mycobacteria of the M. tuberculosis complex (but not coupled to any surrogate assay for diagnosing paratuberculosis and/or detecting one or more mycobacteria of the M. avium complex).

Throughout this specification, unless the context requires otherwise, the word “comprise”, or variations such as “comprises” or “comprising”, will be understood to imply the inclusion of a stated step or element or integer or group of steps or elements or integers but not the exclusion of any other step or element or integer or group of elements or integers.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations or any two or more of said steps or features.

The present invention is not to be limited in scope by the specific examples described herein. Functionally equivalent products, compositions and methods are clearly within the scope of the invention, as described herein.

The present invention is performed without undue experimentation using, unless otherwise indicated, conventional techniques of molecular biology, microbiology, proteomics, virology, recombining DNA technology, peptide synthesis in solution, solid phase peptide synthesis, and immunology. Texts 1-17 infra teaching such conventional techniques are incorporated herein in their entirety by way of reference.

### BRIEF DESCRIPTION OF THE DRAWINGS

FIG. 1 is a graphical representation showing standard curves used for transcript quantification in real time PCR, calculated using genomic DNA as PCR template. Panel A is a standard curve calculated for 16S rRNA transcript using the primer pair rtM.tb16SF (SEQ ID NO: 17) and rtM.tb16SR (SEQ ID NO:18); Panel B is a standard curve calculated for ilvC transcript using the primer pair rtM.tb1ilvCF (SEQ ID NO: 19) and rtM.tbilvCR (SEQ ID NO:20); Panel C is a standard curve calculated for BSX transcript using the primer pair rtM.tbBSXF (SEQ ID NO: 21) and rtM.tbBSXR (SEQ ID NO:22); Panel D is a standard curve calculated for Rv1265 transcript using the primer pair rtM.tbRv1265F (SEQ ID NO: 23) and rtM.tbRv1265R (SEQ ID NO:24); Panel E is a standard curve calculated for S9 transcript using the primer pair rtM.tbS9F (SEQ ID NO: 25) and rtM.tbS9R (SEQ ID NO:26). Regression (R²) and equations for the curves are shown.

FIG. 2 is a graphical representation showing amplified sandwich ELISA standard curves for detection of M. tuberculosis ketol-acid reductoisomerase (KARI). Standard curves were generated using the optimised ELISA conditions for detection of KARI in buffer as described in the examples. The concentration of recombinant KARI protein (pg/ml) is indicated on the X-axis [rilvC] in logarithmic scale, and the mean OD is shown on the Y-axis. The capture and detector antibodies (Mo1283F and Ch34/35, respectively) were used at 5 and 2.5 μg/mL respectively. A 4-parameter logistic equation was used to fit a standard curve to the data for representative ELISA (n=2) (#1217; LOD=1690 pg/mL).

FIG. 3 is a graphical representation showing KARI protein expression (relative to total cellular protein) in one laboratory strain (H37Rv) and two clinical strains (CSU93 and HN878) of M. tuberculosis, as determined by sandwich ELISA. Whole cell lysates (WCL) from M. tuberculosis strain H37Rv (left), M. tuberculosis strains CSU93 (middle) and HN878 (right) were analysed by sandwich ELISA. The concentration of endogenous protein was calculated by interpolation from the standard curve and was corrected for the spiking level. Data were obtained from replicate experiments for which each sample was analyzed in duplicate. The level of endogenous protein (expressed as pg/μg total cell protein) was plotted as mean±SD for each of the three culture strains. M. tuberculosis strains were obtained courtesy of Colorado State University.

FIG. 4 is a graphical representation showing KARI protein expression (relative to total cell protein) in M. tuberculosis, M. intracellulaire and M. avium, as determined by sandwich ELISA. Whole cell lysates from M. tuberculosis strain H37Rv (left), and from M. avium (middle) and M. intracellulaire (right) were assayed in duplicate in two independent experiments. The concentration of endogenous protein was calculated by interpolation from the standard curve and was corrected for dilution factor. The level of endogenous protein expressed as pg/μg total cellular protein was plotted as mean±SD for each of the three Mycobacteria tested.

FIG. 5 is a graphical representation showing KARI protein expression in filtrates obtained from whole cell lysates of M. tuberculosis, M. intracellulaire and M. avium, as determined by sandwich ELISA. Filtrates obtained from whole cell lysates of M. tuberculosis strain H37Rv (left), M. avium (middle) and M. intracellulaire (right) were assayed in duplicate. The concentration of endogenous protein was calculated by interpolation from the standard curve and was corrected for dilution factor (if any). The level of endogenous protein expressed as pg/μL filtrate was plotted as mean±SD for each of the three Mycobacteria.

FIG. 6 is a graphical representation of sandwich ELISA results showing lack of significant cross-reactivity of antibodies against M. tuberculosis KARI protein with 0.1 μg/ml (columns 2, 4, 6) or 100 μg/ml (columns 1, 3, 5) of whole cell lysate from the non-mycobacteria pathogens Escherichia coli (columns 1 and 2), Bacillus subtilis (columns 3 and 4), and Pseudomonas aeruginosa (columns 5 and 6). Whole cell lysates were assayed in duplicate in 2 separate experiments. As a control, purified recombinant KARI protein was present at 0 ng/ml (column 7), 0.12 ng/ml (column 8), 0.49 ng/ml (column 9), 1.95 ng/ml (column 10), 7.8 ng/ml (column 11), 31.3 ng/ml (column 12), or 125 ng/ml (column 13), prepared by serial dilution of recombinant protein in blocking buffer. The mean OD+SD are plotted for the samples and controls.

FIG. 7 is a graphical representation showing the expression of KARI protein in clinical sputa obtained from patients categorized according to their TB smear test results, TB culture test results and HIV status. The series of histograms to the left of the figure show mean OD values for ELISA assays of KARI protein present in calibration standards comprising serial dilutions of M. tuberculosis strain H37Rv whole cell lysates: 60 μg/ml column 1; 20 μg/ml column 2; 6.67 μg/ml column 3; 2.22 μg/ml column 4; 0.74 μg/ml column 5; 0.25 μg/ml column 6; 0.08 μg/ml column 7; 0 μg/ml column 8. The series of histograms to the left of the figure show mean OD values for ELISA assays of KARI protein present in patient samples prepared as described in the accompanying examples (Method 3: 4.5 mL sputum-C1, 17×150 μL replacement amplification ELISA). “MPC” indicates the sample identification code; “smear” indicates smear test result: “cult” indicates the culture test result; and “HIV” indicates HIV status. Open bars indicate smear negative/culture negative samples. Filled bars indicate smear positive/culture positive samples. Data show significantly higher levels of KARI protein cross-reactivity in smear positive/culture positive samples independent of HIV status of the subject.

FIG. 8 is a graphical representation showing the expression in clinical sputa obtained from patients categorized according to their TB smear test results, TB culture test results and HIV status expressed as pg KARI protein/ml sample volume. Data shown in FIG. 6 were converted to pg antigen based on KARI protein calibration values therein which permitted interpolation of ug/mL KARI protein for whole cell extracts of M. tuberculosis H37Rv into pg/mL rKARI protein. “MPC” indicates the sample identification code; “smear” indicates smear test result: “cult” indicates the culture test result; and “HIV” indicates HIV status. Open bars indicate smear negative/culture negative samples. Filled bars indicate smear positive/culture positive samples. Data show significantly higher levels of KARI protein cross-reactivity in smear positive/culture positive samples independent of HIV status of the subject. LOD for assay=˜900 pg/mL.

FIG. 9 provides graphical representations showing inhibition of antibody binding to recombinant KARI (Ilvc) protein by sputum. An amplified ELISA system was used to analyze the degree of inhibition of antibody binding to recombinant protein in TB-negative sputum. The sputum was spiked with 10 ng/mL of each recombinant protein (columns 1-3; note columns 1 & 2 are not visible on graph), a 1 in 3 dilution of the mixture in blocking buffer (columns 4-6), a 1 in 9 dilution of the mixture in blocking buffer (columns 7-9), and a 1 in 27 dilution of the mixture in blocking buffer (columns 10-12). Samples were incubated overnight at 4° C. before assay (columns 1, 4, 7, 10) or assayed immediately (columns 2, 5, 8, 11). Positive control samples lacked sputum and were incubated overnight before assay (columns 3, 6, 9, 12). The samples were assayed in duplicate wells in each of 2 separate experiments. The concentration of recombinant protein detected in sputum at each dilution was calculated by interpolation from the standard curve, and expressed as a % of the spiked concentration of recombinant protein (% signal recovery). The level of recovery was plotted as mean % signal recovery ±SD for each of the 4 dilution factors for the three treatments. Data are presented as percentage signal recovery (Y-axis) for each dilution shown on the x-axis.

FIG. 10 provides graphical representations showing the inhibition of antibody binding to endogenous M. tuberculosis KARI protein by sputum, as determined by amplified sandwich ELISA. An amplified ELISA system was used to analyse the levels of quenching and masking of antibody binding to endogenous KARI protein in whole cell lysates of M. tuberculosis H37Rv spiked into TB-negative sputum. The sputum was spiked with whole cell lysates to achieve target concentrations in sputum of KARI=31 ng/mL (columns 1-3; note columns 1 & 2 are not visible on graph), a 1 in 3 dilution of the mixture in blocking buffer (columns 4-6), a 1 in 9 dilution of the mixture in blocking buffer (columns 7-9), and a 1 in 27 dilution of the mixture in blocking buffer (columns 10-12). Samples were incubated overnight at 4° C. before assay (columns 1, 4, 7, 10) or assayed immediately (columns 2, 5, 8, 11). Positive control samples lacked sputum and were incubated overnight before assay (columns 3, 6, 9, 12). The samples were assayed in duplicate wells in each of 2 separate experiments. The concentration of endogenous protein detected in sputum at each dilution was calculated by interpolation from the standard curve (H37Rv-WCL serially diluted in blocking buffer), and expressed as a % of the spiked concentration (% signal recovery). The level of recovery was plotted as mean % signal recovery for each of the 4 dilution factors for the three treatments.

FIG. 11 is a graphical representation showing relative expression of BSX (columns 1-3), EF-Tu (columns 4-6), KARI (columns 7-9), P5CR (ProC) (columns 10-12), Rv1265 (columns 13-15), S9 (columns 16-18), and TetR (columns 19-21) expressed on the basis of total cellular protein in M. tuberculosis strain H37Rv (first column in each group of 3 columns), CSU93 (second column in each group of 3 columns) and HN878 (third column in each group of 3 columns), as determined by sandwich ELISA. The concentration of endogenous protein was calculated by interpolation from the standard curve and was corrected for the dilution factor. Data were obtained from replicate experiments in which each sample was analysed in duplicate. The levels of endogenous protein (expressed as pg/μg total cell protein) was plotted as mean±SD for each of the TB antigens analysed.

FIG. 12 is an expanded view of the graphical representation set forth in FIG. 11 showing the expression levels for some low expressing antigens.

FIG. 13 is a graphical representation showing relative expression of BSX (columns 1-3), EF-Tu (columns 4-6), KARI (columns 7-9), P5CR (ProC) (columns 10-12Rv1265 (columns 13-15), S9 (columns 16-18), and TetR (columns 19-21) expressed as ng protein per 1×10⁶ CFU M. tuberculosis strain H37Rv (first column in each group of 3 columns), M. avium (second column in each group of 3 columns) and in M. intracellulaire (third column in each group of 3 columns), as determined by sandwich ELISA. The concentration of endogenous protein was calculated by interpolation from the standard curve and was corrected for the dilution factor. Data were obtained from replicate experiments in which each sample was analysed in duplicate. The levels of endogenous protein were plotted as mean±SD for each of the TB antigens analyzed. Data indicate specific expression of BSX, EF-Tu, KARI, Rv1265 and S9 in M. tuberculosis.

FIG. 14 is an expanded view of the graphical representation set forth in FIG. 13 showing the expression levels for some low expressing antigens. Data indicate specific expression of BSX, EF-Tu, P5CR, Rv1265, S9, and TetR in M. tuberculosis with detectable expression of KARI in M. intracellulaire and M. avium at these low detection limits.

FIG. 15 is a graphical representation showing relative expression of BSX (columns 1-3), EF-Tu (columns 4-6), KARI (Ilvc) (columns 7-9), P5CR (ProC) (columns 10-12), Rv1265 (columns 13-15), S9 (columns 16-18), and TetR (columns 19-21) expressed as pg antigen per μg total cell protein of M. tuberculosis strain H37Rv (first column in each group of 3 columns), M. avium (second column in each group of 3 columns) and in M. intracellulaire (third column in each group of 3 columns), as determined by sandwich ELISA. The concentration of endogenous protein was calculated by interpolation from the standard curve and was corrected for the dilution factor. Data were obtained from replicate experiments in which each sample was analysed in duplicate. The levels of endogenous protein were plotted as mean±SD for each of the TB antigens analyzed. Data indicate specific expression of BSX, EF-Tu, Rv1265,S9, and TetR (see FIG. 16) in M. tuberculosis. Detectable expression of KARI was apparent in M. intracellulaire and M. avium under these conditions.

FIG. 16 is an expanded view of the graphical representation set forth in FIG. 15 showing the expression levels for some low expressing antigens. Data indicate specific expression of Rv1265 in M. tuberculosis with detectable expression of most other antigens tested in M. intracellulaire and M. avium at these low detection limits.

FIG. 17 is a graphical representation showing relative expression of BSX (columns 1-3), EF-Tu (columns 4-6), KARI (ilvC) (columns 7-9), P5CR (ProC) (columns 10-12), Rv1265 (columns 13-15), S9 (columns 16-18), and TetR (columns 19-21) expressed as pg antigen per μL filtrate of a cell culture supernatant, i.e., culture filtrate of M. tuberculosis strain H37Rv (first column in each group of 3 columns), M. avium (second column in each group of 3 columns) and in M. intracellulaire (third column in each group of 3 columns), as determined by sandwich ELISA. The concentration of endogenous protein was calculated by interpolation from the standard curve and was corrected for the dilution factor. Data were obtained from replicate experiments in which each sample was analysed in duplicate. The levels of endogenous protein were plotted as mean±SD for each of the TB antigens analyzed.

FIG. 18 is an expanded view of the graphical representation set forth in FIG. 17 showing the expression levels for some low expressing antigens.

FIG. 19 is deleted

FIG. 20 is a graphical representation of transcript levels of ilvC in sputum samples as determined by quantitative real-time PCR. The level of the RNA transcripts of the ilvC gene as expressed in clinical patients was determined in samples isolated from patient samples. Nucleic acid was isolated, cDNA prepared, and expression determined using real-time PCR. Values are in copy/μg cDNA.

FIG. 21 is a graphical representation of transcript levels of ilvC compared to 16S rRNA in sputum samples as determined by quantitative real-time PCR, and compared to smear status. The level of the RNA transcripts of the ilvC gene and of 16S rRNA was determined in samples isolated from patient samples. Nucleic acid was isolated, cDNA prepared, and expression determined using real-time PCR. Values are in copy/μg cDNA.

FIG. 22 is a graphical representation of the comparative detection of recombinant protein and M.tb. by four target assays. Whole cell lysate of cultured cells of the M.tb. strain H37Rv were prepared and material was serially diluted in PBS-T and applied to ELISA using conditions as used for screening clinical sputum as described herein.

FIG. 23 is a graphical representation of the cross-reactivity determination for anti-KARI Mo2B1 for common micro-organisms. Whole cell lysate of cultured cells of the M.tb. strain H37Rv were prepared and material was serially diluted in PBS-T and applied to ELISA using conditions as used for screening clinical sputum as described herein.

FIG. 24 is a graphical representation of the KARI protein in sub-cellular fractions of cell wall and cell membrane of M.tb. Subcellular fractions of M.tb. strain H3tRv was serially diluted in PBS-T for application to the ELISA using conditions as used for screening clinical sputum as described herein.

FIG. 25 is a graphical representation of a standard curve of M.tb. in buffer with KARI protein and a point-of-care assay format (DiagnostIQ). Chaotropic equilibration buffer was spiked with M.tb. (H37Rv WCL) ranging from 7.5-120 μg/mL. 500 μL of each sample was added to DiagnostIq test and detected with a old conjugated monoclonal 2B1. Endogenous KARI was captured with chicken anti-KARI (Ch34/35). Each assay point was performed in duplicate. 5 μg H37Rv WCL is approx 2 ng of recombinant KARI (ilvC).

FIG. 26 is a graphical representation of screening results of M.tb. in clinical sputum samples using KARI as target antigen and antibody Mo1283F. Well characterized clinical samples representing smear negative, 2+, and 3+ individuals, including both HIV positive and negative patients were analysed over a serial dilution series. Results employing antibody 1283F as detector show detection of ilvC in six out of six smear positive clinical samples in ELISA format.

FIG. 27 is a graphical representation of M.tb. in clinical sputum samples using KARI as target antigen and optimised Mo2B1 monoclonal antibody. Well characterized clinical samples representing smear negative, 2+, and 3+ individuals, including both HIV positive and negative patients were analysed over a serial dilution series. Control sputa were also run through the sample process with and without a 10 □g/mL M.tb. WCL spike at time of solubilisation. 12 samples were run per ELISA, with each sample analysed at serial dilution in replicate (triplicates were employed where dilutions of 1/1 and 1/3 only were assayed, and duplicates where 1/1, 1/3, and 1/9 dilution are shown. MPC denotes samples sourced from Cameroon.

FIG. 28 is a photographic representation of the detection of rilvC (rKARI) and endogenous ilvC (KARI) by western blot analysis. Recombinant KARI protein (rilvC), and whole cell lysate of cultured M.tb. (WCL H37Rv) were loaded onto SDS-PAGE at 10 ng/lane and 10 □g/lane respectively. Blots were probed with primary antibody in the presence and absence of excess recombinant protein.

FIG. 29 is a photographic representation of the detection of rilvC (rKARI) and endogenous ilvC (KARI) by wester blot analysis. MW markers, recombinant ilvC, and whole cell lysate of cultured M.tb. (WCL H37Rv) were loaded onto SD S-PAGE at 1 ng/lane and 5 mg/lane respectively in lanes 1-3. Blaots were probed with primary antibody. Ch34/35 from original chicken antibody pool, Ch35 was subsequently re-boosted with a highly purified preparation of recombinant ilvC. Mo2B1, Mo1E7, Mo2C7 and Mo3A2 are monoclonal antibodies which bind to regions 1, 2, 5, and 6 of the KARI peptide backbone respectively (cf FIG. 36).

FIG. 30 is a graphical representation showing specificity of KARI (ilvC) ELISA format. Whole cell lysates of cultured cells were prepared and serially diluted in PBS-T for application to the ELISA under conditions identical to those employed for standards in the screening of clinical sputum. No significant cross-reactivity was detected for other common bacterial targets: E. coli, Pseudomonas aeruginosa, Bacillus subtillus, and Saccharomyces cerevisiae.

FIG. 31 is a graphical representation of the detection of KARI (ilvC) in clinical samples using Mo2B1 monoclonal antibody capture assay. Clinical samples (n>25) were processed as described herein. Representative Data are shown above for a subset of clinical samples screened.

### DETAILED DESCRIPTION OF THE PREFERRED EMBODIMENTS

### Detection Means

The detection means of the method provides for high sensitivity and/or high specificity and/or high reproducibility e.g., compared to conventional methods, and/or other nucleic acid amplification methods known in the art.

The term “high sensitivity” is taken to mean detection about 100% detection of ilvC nucleic acid(s) in a sample; or about 99% detection of ilvC nucleic acid(s) in a sample; or about 98% detection of ilvC nucleic acid(s) in a sample; or about 97% detection of ilvC nucleic acid(s) in a sample; or about 96% detection of ilvC nucleic acid(s) in a sample; or about 95% detection of ilvC nucleic acid(s) in a sample; or about 94% detection of ilvC nucleic acid(s) in a sample; or about 93% detection of ilvC nucleic acid(s) in a sample; or about 92% detection of ilvC nucleic acid(s) in a sample; or about 91% detection of ilvC nucleic acid(s) in a sample; or about 90% detection of ilvC nucleic acid(s) in a sample; or about 89% detection of ilvC nucleic acid(s) in a sample; or about 88% detection of ilvC nucleic acid(s) in a sample; or about 87% detection of ilvC nucleic acid(s) in a sample; or about 86% detection of ilvC nucleic acid(s) in a sample; or about 85% detection of ilvC nucleic acid(s) in a sample; or about 80 to about 84% detection of ilvC nucleic acid(s) in a sample.

In one example, the detection means detects ilvC nucleic acid is about 15-20 times more sensitive than known methods. In another example, the method of the invention detects ilvC nucleic acid is about 20 to 30 times more sensitive than known methods. In another example, the method of the invention detects ilvC nucleic acid is about 30 to 50 times more sensitive than known methods. In another example, the method of the invention detects ilvC nucleic acid is about 2 to 15 times more sensitive than known methods. In another example, the method of the invention detects ilvC nucleic acid is about 5 to 10 times more sensitive than known methods.

The term “high specificity” is taken to mean about 100% specificity for ilvC nucleic acid detection; or about 99% specificity for ilvC nucleic acid detection; or about 98% specificity for ilvC nucleic acid detection; or about 97% specificity for ilvC nucleic acid detection; or about 96% specificity for ilvC nucleic acid detection; or about 95% specificity for ilvC nucleic acid detection; or about 80% to about 95% specificity for ilvC nucleic acid detection; or about 80% to about 85% specificity for ilvC nucleic acid detection.

In one example, the detection means according to any embodiment described herein, reproducibly detects at least about 1 copy of template/μg of starting template. In another example, the method of the invention according to any embodiment described herein, reproducibly detects between about 1 to 2000 copies of template/μg of starting template. In another example, the method of the invention according to any embodiment described herein detects between about 1 to 1000 copies of template/μg of starting template. In another example, the method of the invention according to any embodiment described herein detects between 1 to 500 of template/μg of starting template. In another example, the method of the invention according to any embodiment described herein detects between about 1 to 400 copies of template/μg of starting template. In another example, the method of the invention according to any embodiment described herein detects between about 1 to 300 copies of template/μg of starting template. In another example, the method of the invention according to any embodiment described herein detects between about 1 to 200 copies of template/μg of starting template. In another example, the method of the invention according to any embodiment described herein detects between about 1 to 100 copies of template/μg of starting template. In another example, the method of the invention according to any embodiment described herein detects between about 1 to 50 copies of template/μg of starting template.

The detection means employed in the inventive method may comprise a nucleic acid hybridization employing a probe or peptide nucleic acid (PNA) or locked nucleic acid (LNA) probe) to detect input nucleic acid e.g., by producing a detectable signal. Alternatively, the detection means employed in the inventive method may comprise an amplification reaction e.g., an amplification reaction requiring thermal cycling or amplification reaction that does not require thermal cycling, employing one or more primers and optionally, one or more probes as described herein, to thereby produce an amplicon comprising ilvC nucleic acid derived from the input nucleic acid.

### Input Nucleic Acid or Template

The nucleic acid amplification-based assays as described in any example hereof are for detecting and/or quantitating ilvC nucleic acid(s) (ilvC-NAA) in clinical samples at high sensitivity and/or high selectivity and reproducibility amongst a multiplicity of samples from the same subject. The ilvC-NAA based tests also provide reproducible results across a large cohort of clinical samples, e.g., ilvC transcript level correlating with severity of infection and/or disease progression. For example, this may be achieved by detection of amplified nucleic acid and quantitation.

The “template” is taken to be any ilvC nucleic acid/s and may comprise DNA, RNA or RNA/DNA with or without any nucleotide analogs therein including single-stranded or double-stranded genomic DNA, mRNA or cDNA. However, it is preferred that the template is a cDNA produced from an mRNA transcript of the ilvC gene.

In one example, the template is a nucleic acid encoding the KARI protein as set forth in SEQ ID NO: 1. In another example the template is a nucleic acid encoding a protein that is at least 80% homologous to the KARI protein as set forth in SEQ ID NO: 1. In another example the template is a nucleic acid encoding a protein that is between 80% to 100% homologous to the KARI protein as set forth in SEQ ID NO: 1. In another example the template is a nucleic acid encoding a protein that is between 85% to 100% homologous to the KARI protein as set forth in SEQ ID NO: 1. In another example the template is a nucleic acid encoding a protein that is between 90% to 100% homologous to the KARI protein as set forth in SEQ ID NO: 1. In another example the template is a nucleic acid encoding a protein that is between 95% to 100% homologous to the KARI protein as set forth in SEQ ID NO: 1.

In another example, the template is a nucleotide coding sequence as set forth in SEQ ID NO: 2. In another example the template is a nucleotide coding sequence that is at least 80% homologous to the coding sequence as set forth in SEQ ID NO: 2. In another example the template is a nucleotide coding sequence that is at least 80% to 100% homologous to the coding sequence as set forth in SEQ ID NO: 2. In another example the template is a nucleotide coding sequence that is at least 85% to 100% homologous to the coding sequence as set forth in SEQ ID NO: 2. In another example the template is a nucleotide coding sequence that is at least 90% to 100% homologous to the coding sequence as set forth in SEQ ID NO: 2. In another example the template is a nucleotide coding sequence that is at least 95% to 100% homologous to the coding sequence as set forth in SEQ ID NO: 2. In another example the template is a nucleotide coding sequence that is at least 85% to 100% homologous to the coding sequence as set forth in SEQ ID NO: 2.

It is to be understood in this context that the stated proteins and encoding sequences supra include homologs of the exemplified proteins and encoding sequences exemplified by way of the Sequence Listing, wherein said homologs are expressed by one or a plurality of bacteria of the M. tuberculosis complex e.g., M. tuberculosis and/or M. bovis and/or M. africanum and/or M. canetti and/or M. microti.

In another example, the specific amplification of an ilvC nucleic acid of M. tb or M.tb complex in accordance with the invention utilizes a nucleic acid concentration in a standard 25 μl reaction of less than about 10 ng or less than about 5 ng or less than 4 ng or less than 3 ng or less than 2 ng or less than 1 ng or less than about 500 pg or less than about 100 pg or less than about 50 pg, excluding any contribution from a primer or probe. Preferred nucleic acid concentrations in a standard 25 μl reaction are at about 10 pg or 15 pg or 20 pg or 25 pg or 30 pg or 35 pg or 40 pg or 45 pg or 50 pg, such as e.g., about 35 pg to about 45 pg excluding any contribution from a primer or probe. Lower concentrations of input nucleic acid (excluding any contribution from a primer or probe) may reduce non-specific amplification effects such as amplification of M. avium sequence for certain primer pairs described herein. A skilled artisan will be able to adjust these nucleic acid concentrations for different reaction conditions without undue experimentation or effort.

### Primer Design

As will be known to the skilled artisan, a “primer” is a nucleic acid molecule comprising any combination of ribonucleotides, deoxyribonucleotides and analogs thereof such that it comprises DNA, RNA or DNA/RNA with one or more ribonucleotide or deoxyribonucleotide analogs contained therein, and capable of annealing to a nucleic acid template to act as a binding site for an enzyme, e.g., DNA or RNA polymerase, thereby providing a site for initiation of replication of a specific nucleic acid in the 5′ to 3′ direction. The nucleotide sequence of a primer is generally substantially complementary to the nucleotide sequence of a template nucleic acid to be amplified, or at least comprises a region of complementarity sufficient for annealing to occur and extension in the 5′ to 3′ direction there from. However, as will be apparent to the skilled artisan a degree of non-complementarity will not significantly adversely affect the ability of a primer to initiate extension. Suitable methods for designing and/or producing a primer suitable for use in the method of the invention are known in the art and/or described herein. Primers are generally, but not necessarily, short synthetic nucleic acids of about 12-50 nucleotides in length. Preferably, the first primer or each primer of the set of first primers comprises at least about 12-15 nucleotides in length capable of annealing to a strand of the nucleic acid template. Primers may also comprise at least about 20 or 25 or 30 nucleotides in length capable of annealing to a strand of the template.

As will be apparent to the skilled artisan, the number of nucleotides capable of annealing to a nucleic acid template is related to the stringency under which the primer will anneal. Preferably, a primer for use in the method of the invention anneals to a nucleic acid template under moderate to high stringency conditions.

In one embodiment, the stringency under which a primer of the invention anneals to a template nucleic acid is determined empirically. In another example, the conditions under which a primer anneals to a nucleic acid template are determined in silico. In another example, the nearest neighbour method described by Breslauer et al., Proc. Natl. Acad. Sci. USA, 83:3746-3750, 1986 is useful for determining the Tm of a primer. Other exemplary methods for determining the Tm of a primer are described herein.

The design of primers for the specific detection and sensitive detection of ilvC nucleic acid/s are described herein. For example, the primers and the putative amplified product may be screened to ensure specificity by performing a BLAST search as exemplified herein. Accordingly, primers with high homology across the M.tb. complex, however showing no significant homology with other mycobacteria are disclosed. In one embodiment, a primer is designed such that it comprises a sequence having at least about 80% identity overall to a region of a strand of a template nucleic acid. More preferably, the degree of sequence identity is at least about 80% or 85% or 90% or 95% or 98% or 99% or 100%. For example, the primer or a region of a primer may comprise a sequence having at least about 80% identity to a region of strand of a template of interest e.g., ilvC or other M.tb complex sequence.

In one embodiment, a primer is designed such that it comprises a sequence having at least about 80% identity overall to a strand of a template nucleic acid, e.g., an ilvC transcript in the case of a single analyte NAA-based assay of the invention, or any one of the other transcripts as described in the multi-analyte tests, e.g., a BSX transcript. More preferably, the degree of sequence identity to the template is at least about 85% or 90% or 95% or 98% or 99%. For example, the primer or a region of a primer may comprise a sequence having at least about 80% identity to a strand of a template of nucleic acid.

Clearly, the specific composition of a primer of the invention will depend upon the sequence of the nucleic acid of interest. In particular, the sequence need only be sufficient to allow for annealing of the primer to a template nucleic acid and initiation of an amplification reaction.

In the present context, the term “annealing” or similar term shall be taken to mean that a primer and a nucleic acid to be amplified (i.e., template or primary amplification product) are base-paired to each other to form a double-stranded or partially double-stranded nucleic acid, using a temperature or other reaction condition known in the art to promote or permit base-pairing between complementary nucleotide residues. As will be known to the skilled artisan, the ability to form a duplex and/or the stability of a formed duplex depend on one or more factors including the length of a region of complementarity between the primer and nucleic acid to be amplified, the percentage content of adenine and thymine in a region of complementarity (i.e., “A+T content”), the incubation temperature relative to the melting temperature (Tm) of a duplex, and the salt concentration of a buffer or other solution in which the amplification is performed. Generally, to promote annealing, the primers and nucleic acid to be amplified are incubated at a temperature that is at least about 1-5° C. below a primer Tm that is predicted from its A+T content and length. Duplex formation can also be enhanced or stabilized by increasing the amount of a salt (e.g., NaCl, MgCl₂, KCl, sodium citrate, etc) in the reaction buffer, or by increasing the time period of the incubation, as described by Sambrook et al., Molecular Cloning: A Laboratory Manual, Cold Spring Harbor Laboratory Press; Hames and Higgins, Nucleic Acid Hybridization: A Practical Approach, IRL Press, Oxford (1985); Berger and Kimmel, Guide to Molecular Cloning Techniques, In: Methods in Enzymology, Vol 152, Academic Press, San Diego Calif. (1987); or Ausubel et al., Current Protocols in Molecular Biology, Wiley Interscience, ISBN 047150338 (1992).

As a primer is generally extended in the 5′- to 3′-direction it is preferred that at least the 3′-terminal nucleotide is complementary to the relevant nucleotide in the template nucleic acid. More preferably, at least the 3 or 4 or 6 or 8 or 10 contiguous nucleotides at the 3′-terminus of the primer are complementary to the relevant nucleotides in the template nucleic acid. The complementarity of the 3′ terminus of the primer ensures that the extending end of the primer is capable of initiating amplification of the template nucleic acid, for example, by a polymerase.

As regions of non-complementarity reduce the predicted Tm of a primer and may be associated with amplification of non-template nucleic acid it is preferred that a primer of the invention does not comprise multiple contiguous nucleotides that are not identical to a strand of the template nucleic acid. Preferably, the primer comprises no more than 6 or 5 or 4 or 3 or 2 contiguous nucleotides that are not identical to a strand of the template nucleic acid. More preferably, any nucleotides that are not identical to a strand of the template nucleic acid are non-contiguous.

To determine whether or not two nucleotide sequences fall within a particular percentage identity limitation recited herein, those skilled in the art will be aware that it is necessary to conduct a side-by-side comparison or multiple alignment of sequences. In such comparisons or alignments, differences may arise in the positioning of non-identical residues, depending upon the algorithm used to perform the alignment. In the present context, reference to a percentage identity between two or more nucleotide sequences shall be taken to refer to the number of identical residues between said sequences as determined using any standard algorithm known to those skilled in the art. For example, nucleotide sequences may be aligned and their identity calculated using the BESTFIT program or other appropriate program of the Computer Genetics Group, Inc., University Research Park, Madison, Wis., United States of America (Devereaux et al, Nucl. Acids Res. 12, 387-395, 1984).

Alternatively, a suite of commonly used and freely available sequence comparison algorithms is provided by the National Center for Biotechnology Information (NCBI) Basic Local Alignment Search Tool (BLAST) (Altschul et al. J. Mol. Biol. 215: 403-410, 1990), which is available from several sources, including the NCBI, Bethesda, Md. The BLAST software suite includes various sequence analysis programs including “blastn,” that is used to align a known nucleotide sequence with other polynucleotide sequences from a variety of databases. Also available is a tool called “BLAST 2 Sequences” that is used for direct pairwise comparison of two nucleotide sequences.

As used herein the term “NCBI” shall be taken to mean the database of the National Center for Biotechnology Information at the National Library of Medicine at the National Institutes of Health of the Government of the United States of America, Bethesda, Md., 20894.

Generally, a primer comprises or consists of at least about 10 nucleotides, more preferably at least about 12 nucleotides or at least about 15 or 20 nucleotides that anneal to a nucleic acid template or are complementary to the nucleic acid template. However, longer primers are also used in amplification reactions, for example, reactions in which a long region of nucleic acid (e.g., greater than 1000 bp) is amplified. Accordingly, the present invention additionally contemplates a primer comprising at least about 25 or 30 or 35 nucleotides that anneal to a nucleic acid template or are complementary to the nucleic acid template.

Alternatively, a primer comprising one or modified bases, such as, for example, locked nucleic acid (LNA) or peptide nucleic acid (PNA) need only comprise a region of at least about 8 nucleotides that anneal to a nucleic acid template or are complementary to the nucleic acid template. Preferably, the complementary nucleotides are contiguous.

As will be apparent to the skilled artisan, the number of nucleotides capable of annealing to a nucleic acid template is related to the stringency under which the primer will anneal. Preferably, a primer of the invention anneals to a nucleic acid template under moderate to high stringency conditions.

In one embodiment, the stringency under which a primer of the invention anneals to a template nucleic acid is determined empirically. Generally, such a method requires performance of an amplification reaction using one or more primers under various conditions and determining the level of specific amplification produced.

Alternatively, a primer of the invention is labelled with a detectable marker (e.g., a radionucleotide or a fluorescent marker) and the level of primer that has annealed to a target nucleic acid under suitably stringent conditions is determined.

For the purposes of defining the level of stringency, a moderate stringency annealing conditions will generally be achieved using a condition selected from the group consisting of:
(i) an incubation temperature between about 42° C. and about 55° C.;
(ii) an incubation temperature between about 15° C. and 10° C. less than the predicted Tm for a primer; and
(iii) a Mg²⁺ concentration of between about 2 mM and 3 mM.

High stringency annealing conditions will generally be achieved using a condition selected from the group consisting of:
(i) an incubation temperature above about 55° C. and preferably above about 65° C.;
(ii) an incubation temperature between about 10° C. and 1° C. less than the predicted Tm for a primer; and
(iii) a Mg²⁺ concentration of between about 1 mM and 1.9 mM.

Alternative or additional conditions for enhancing stringency of annealing will be apparent to the skilled artisan. For example, a reagent such as, for example, glycerol (5-10%), DMSO (2-10%), formamide (1-5%), Betaine (0.5-2M) or tetramethylammonium chloride (TMAC, >50 mM) are known to alter the annealing temperature of a primer and a nucleic acid template (Sarkar et al., Nucl. Acids Res. 18: 7465; 1990, Baskaran et al. Genome Res. 6: 633-638, 1996; and Frackman et al., Promega Notes 65: 27, 1998).

Conditions for altering the stringency of an amplification reaction are understood by those skilled in the art. For the purposes of further clarification only, reference to the parameters affecting annealing between nucleic acid molecules is found in Ausubel et al. (Current Protocols in Molecular Biology, Wiley Interscience, ISBN 047150338, 1992), which is herein incorporated by reference.

Alternatively, the conditions under which a primer anneals to a nucleic acid template are determined in silico. For example, methods for determining the predicted melting temperature (or Tm) of a primer (or the temperature at which a primer denatures from a specific nucleic acid) are known in the art.

For example, the method of Wallace et al., (Nucleic Acids Res. 6, 3543, 1979) estimates the Tm of a primer based on the G, C, T and A content. In particular, the described method uses the formula 2 (A+G)+4(G+C) to estimate the Tm of a probe or primer.

Alternatively, the nearest neighbour method described by Breslauer et al., Proc. Natl. Acad. Sci. USA, 83:3746-3750, 1986 is useful for determining the Tm of a primer. The nearest neighbour method uses the formula:

Tₘ(calc)=ΣΔH⁰/(R1n(Cₜ/n)+ΣΔS⁰),

wherein ΔH⁰ is standard enthalpy for helix formation, ΔS⁰ is standard entropy for helix formation, C_{f} is the total strand concentration, n reflects the symmetry factor, which is 1 in the case of self-complementary strands and 4 in the case of non-self-complementary strands and R is the gas constant (1.987).

Ryuchlik et al., Nucl. Acids Res. 18: 6409-6412, 1990 described an alternative formula for determining Tm of an oligonucleotide:

$${Tm}^{primer} = \frac{dH}{dS + R ln ( c / 4 )} + 16.61 g \frac{[ {K}^{+} ]}{1 + 0.7 [ {K}^{+} ]} - 273.15 ,$$

wherein, dH is enthalpy for helix formation, dS is entropy for helix formation, R is molar gas constant (1.987 cal/° C. mol), “c” is the nucleic acid molar concentration (determined empirically, W. Rychlik et. al., supra), (default value is 0.2 μM for unified thermodynamic parameters), [K⁺] is salt molar concentration (default value is 50 mM).

Suitable software for determining the Tm of an oligonucleotide using the nearest neighbour method is known in the art and available from, for example, US Department of Commerce, Northwest Fisheries Service Center and Department of Molecular Genetics and Biochemistry, University of Pittsburgh School of Medicine.

Alternatively, for longer primers (i.e., a primer comprising at least about 200 nucleotides), the method of Meinkoth and Wahl (In: Anal Biochem, 138: 267-284, 1984), is useful for determining the Tm of the primer. This method uses the formula:

81.5+16.6(log₁₀M)+0.41(% GC)−0.61(% form)−500/Length in bp,

wherein M is the molarity of Na⁺ and % form is the percentage of formamide (set to 50%)

For a primer that comprises or consists of PNA the Tm is determined using the formula (described by Giesen et al., Nucl. Acids Res., 26: 5004-5006):

T_{mpred}=c_{O}+c₁*T_{mnnDNA}+c₂*f_{pyr}+c₃*length,

wherein, in which T_{mnnDNA} is the melting temperature as calculated using a nearest neighbour model for the corresponding DNA/DNA duplex applying ΔH⁰ and ΔS⁰ values as described by SantaLucia et al. Biochemistry, 35: 3555-3562, 1995. f_{pyr} denotes the fractional pyrimidine content, and length is the PNA sequence length in bases. The constants are c₀=20.79, c₁=0.83, c₂=−26.13 and c₃=0.44

To determine the Tm of a primer comprising one or more LNA residues a modified form of the formula of SantaLucia et al. Biochemistry, 35: 3555-3562, 1995 is used:

$$Tm = \frac{Δ H}{Δ S + ln ( {[ Na ]}^{0.36} * {( C / 4 )}^{1.987} )} ,$$

A suitable program for determining the Tm of a primer comprising LNA is available from, for example, Exiqon, Vedbaek, Germany.

A temperature that is similar to (e.g., within 5° C. or within 10° C.) or equal to the proposed/estimated temperature at which a primer denatures from a template nucleic acid is considered to be high stringency. Medium stringency is to be considered to be within 10° C. to 20° C. or 10° C. to 15° C. of the calculated Tm of the probe or primer.

Preferably, a primer of the invention selectively anneals to a target nucleic acid. The term “selectively anneals” means that the probe is used under conditions where a target nucleic acid, anneals to the probe to produce a signal that is significantly above background (i.e., a high signal-to-noise ratio). The level of specificity of annealing is determined, for example, by performing an amplification reaction using the primer and detecting the number of different amplicons produced. By “different amplicons” is meant that amplified nucleic acids of differing nucleotide sequence and/or molecular weight are produced. Clearly, amplicons that differ in molecular weight are readily identified, for example, using gel electrophoresis. A primer that selectively anneals to a target nucleic acid produces an amplicon at a level greater than any other amplicon. Preferably only one amplicon is produced at a detectable level.

An alternative technique to determine the selective annealing of a primer of the invention comprises performing a search of known nucleotide sequences from the sample being assayed (e.g., a database of known sequences from an organism or cell from which the template nucleic acid is derived). Using this technique a sequence similar to or complementary to the sequence of the primer is identified. While such a technique does not ensure selective annealing it is useful for determining a primer (or set of primers) capable of annealing to a plurality of sites in a nucleic acid and possibly producing multiple amplicons (i.e., non-selective annealing).

A primer or primer sequence that is predicted to be or shown to be capable of selectively annealing to a nucleic acid template is also optionally analyzed for one or more additional characteristics that make it suitable for use as a primer in the method of the invention. For example, a primer is analyzed to ensure that it is unlikely to form secondary structures (i.e., the primer does not comprise regions of self-complementarity).

Furthermore, should the primer be proposed to be used in a reaction with one or more other primers, all primers may be assessed to determine their ability to anneal to one another and form “primer dimers”. Methods for determining a primer that is capable of self-dimerization and/or primer dimer formation are known in the art and/or described supra.

Methods for designing and/or selecting a primer suitable for use in an amplification reaction are known in the art and described, for example, in Innis and Gelfand (1990) (In: Optimization of PCRs. pp. 3-12 in: PCR Protocols (Innis, Gelfand, Sninsky and White, eds.); Academic Press, New York) and Dieffenbach and Dveksler (Eds) (In: PCR Primer: A Laboratory Manual, Cold Spring Harbour Laboratories, NY, 1995). Such methods are particularly suited, for example, for designing a target specific sequence of a primer of the invention. As used herein target is taken to mean a region spanning at least one nucleotide of a template of interest.

Generally, it is recommended that a primer satisfies the following criteria:
(i). the primer comprises a region that is to anneal to a target sequence having at least about 17-28 bases in length;
(ii). the primer comprises about 50-60% (G+C);
(iii) the 3′-terminus of the primer is a G or C, or CG or GC (this prevents “breathing” of ends and increases efficiency of initiation of amplification);
(iv) preferably, the primer has a Tm between about 55 and about 80° C.;
(v) the primer does not comprise three or more contiguous Cs and/or Gs at the 3′-ends of primers (as this may promote mispriming at G or C-rich sequences due to the stability of annealing);
(vi) the 3′-end of a primer should not be complementary with another primer in a reaction; and
(vii) the primer does not comprise a region of self-complementarity.

Several software programs are available that enable the design of one or more primers, or a region of a primer. For example, a program selected from the group consisting of:
(i) Primer 3, available from the Center for Genome Research, Cambridge, Mass., USA, designs one or more primers for use in an amplification reaction based upon a known template sequence;
(ii) Primer Premier 5, available from Biosoft International, Palo Alto, Calif., USA, designs and/or analyzes primers;
(iii) CODEHOP, available from Fred Hutchinson Cancer Research Centre, Seattle, Wash., USA, designs primers based on multiple protein alignments; and
(iv) FastPCR, available from Institute of Biotechnology, University of Helsinki, Finland, designs multiple primers, including primers for use in a multiplex reaction, based on one or more known sequences.

When designing a primer of the invention, the composition of the template nucleic acid is considered (i.e. the nucleotide sequence) as is the type of amplification reaction to be used.

In designing primers for the specific detection and sensitive detection of ilvC nucleic acid/s, the above criteria are employed. The primers may and the putative amplified product may then be screened to ensure specificity by performing a BLAST search as exemplified herein. In this manner, primers with high homology across the M.tb. complex, with no other mycobacteria showing significant homology may be designed. It will be apparent to a person skilled in the art that the same methods may be used to design primers for the mutli-analyte tests, e.g, for detection of BSX, S9 etc., transcripts.

### Primer Synthesis

Following design and or analysis a specific the primer is produced and/or synthesized. Methods for producing/synthesizing a primer of the present invention are known in the art. For example, oligonucleotide synthesis is described, in Gait (Ed) (In: Oligonucleotide Synthesis: A Practical Approach, IRL Press, Oxford, 1984). For example, a probe or primer may be obtained by biological synthesis (eg. by digestion of a nucleic acid with a restriction endonuclease) or by chemical synthesis. For short sequences (up to about 100 nucleotides) chemical synthesis is preferable.

In one embodiment, a nucleotide comprising deoxynucleotides (e.g., a DNA based oligonucleotide) is produced using standard solid-phase phosphoramidite chemistry. Essentially, this method uses protected nucleoside phosphoramidites to produce a short oligonucleotide (i.e., up to about 80 nucleotides). Typically, an initial 5′-protected nucleoside is attached to a polymer resin by its 3′-hydroxy group. The 5′ hydroxyl group is then de-protected and the subsequent nucleoside-3′-phosphoramidite in the sequence is coupled to the de-protected group. An internucleotide bond is then formed by oxidizing the linked nucleosides to form a phosphotriester. By repeating the steps of de-protection, coupling and oxidation an oligonucleotide of desired length and sequence is obtained. Suitable methods of oligonucleotide synthesis are described, for example, in Caruthers, M. H., et al., “Methods in Enzymology,” Vol. 154, pp. 287-314 (1988).

Other methods for oligonucleotide synthesis include, for example, phosphotriester and phosphodiester methods (Narang, et al. Meth. Enzymol 68: 90, 1979) and synthesis on a support (Beaucage, et al Tetrahedron Letters 22: 1859-1862, 1981), and others described in “Synthesis and Applications of DNA and RNA,” S. A. Narang, editor, Academic Press, New York, 1987, and the references contained therein.

For longer sequences standard replication methods employed in molecular biology are useful, such as, for example, the use of M13 for single stranded DNA as described by J. Messing (1983) Methods Enzymol, 101, 20-78.

Alternatively, a plurality of primers are produced using standard techniques, each primer comprising a portion of a desired primer and a region that allows for annealing to another primer. The primers are then used in an overlap extension method that comprises allowing the primers to anneal and synthesizing copies of a complete primer using a polymerase. Such a method is described, for example, by Stemmer et al., Gene 164, 49-53, 1995.

As discussed supra a primer of the invention, or for use in the method of the invention may also include one or more nucleic acid analogs. For example, a primer comprises a phosphate ester analog and/or a pentose sugar analog. Alternatively, or in addition, a primer of the invention comprises polynucleotide in which the phosphate ester and/or sugar phosphate ester linkages are replaced with other types of linkages, such as N-(2-aminoethyl)-glycine amides and other amides (see, e.g., Nielsen et al., Science 254: 1497-1500, 1991; WO 92/20702; and U.S. Pat. No. 5,719,262); morpholinos (see, for example, U.S. Pat. No. 5,698,685); carbamates (for example, as described in Stirchak & Summerton, J. Org. Chem. 52: 4202, 1987); methylene (methylimino) (as described, for example, in Vasseur et al., J. Am. Chem. Soc. 114: 4006, 1992); 3′-thioformacetals (see, for example, Jones et al., J. Org. Chem. 58: 2983, 1993); sulfamates (as described, for example in, U.S. Pat. No. 5,470,967); 2-aminoethylglycine, commonly referred to as PNA (see, for example, WO 92/20702). Phosphate ester analogs include, but are not limited to, (i) C₁-C₄ alkylphosphonate, e.g. methylphosphonate; (ii) phosphoramidate; (iii) C₁-C₆ alkyl-phosphotriester; (iv) phosphorothioate; and (v) phosphorodithioate. Methods for the production of a primer comprising such a modified nucleotide or nucleotide linkage are known in the art and discussed in the documents referred to supra.

For example, a probe or primer of the invention comprises one or more LNA and/or PNA residues. Probes or primers comprising one or more LNA or PNA residues have been previously shown to anneal to nucleic acid template at a higher temperature than a probe or primer that comprises substantially the same sequence but does not comprise the LNA or PNA residues. Furthermore, incorporation of LNA into a probe or primer has been shown to result in increased signal produced in reactions in which the level of the probe or primer is limiting (Latorra et al., Mol. Cell Probes 17: 253-259, 2003).

Methods for the synthesis of an oligonucleotide comprising LNA are described, for example, in Nielsen et al, J. Chem. Soc. Perkin Trans., 1: 3423, 1997; Singh and Wengel, Chem. Commun. 1247, 1998. Methods for the synthesis of an oligonucleotide comprising are described, for example, in Egholm et al., Am. Chem. Soc., 114: 1895, 1992; Egholm et al., Nature, 365: 566, 1993; and Orum et al., Nucl. Acids Res., 21: 5332, 1993.

Exemplary Primers for Amplifying ilvC Nucleic Acid

This invention also provides sets of amplification primers for use in providing highly-specific amplification of ilvC gene or mRNA sequences from M. tuberculosis or M. tuberculosis complex organisms. In one example, a forward primer has the nucleotide sequence as set forth in SEQ ID NO: 19, and a reverse primer has the nucleotide sequence as set forth in SEQ ID NO: 20. In another example, a forward primer has the nucleotide sequence as set forth in SEQ ID NO: 27, and a reverse primer has the nucleotide sequence as set forth in SEQ ID NO: 28. In another example, a forward primer has the nucleotide sequence as set forth in SEQ ID NO: 29, and a reverse primer has the nucleotide sequence as set forth in SEQ ID NO: 30. In another example, a forward primer has the nucleotide sequence as set forth in SEQ ID NO: 31, and a reverse primer has the nucleotide sequence as set forth in SEQ ID NO: 32. In another example, a forward primer has the nucleotide sequence as set forth in SEQ ID NO: 27, and a reverse primer has the nucleotide sequence as set forth in SEQ ID NO: 20. In another example, a forward primer has the nucleotide sequence as set forth in SEQ ID NO: 27, and a reverse primer has the nucleotide sequence as set forth in SEQ ID NO: 30. In another example, a forward primer has the nucleotide sequence as set forth in SEQ ID NO: 27, and a reverse primer has the nucleotide sequence as set forth in SEQ ID NO: 32. In another example, a forward primer has the nucleotide sequence as set forth in SEQ ID NO: 29, and a reverse primer has the nucleotide sequence as set forth in SEQ ID NO: 20. In another example, a forward primer has the nucleotide sequence as set forth in SEQ ID NO: 29, and a reverse primer has the nucleotide sequence as set forth in SEQ ID NO: 32. In another example, a forward primer has the nucleotide sequence as set forth in SEQ ID NO: 31, and a reverse primer has the nucleotide sequence as set forth in SEQ ID NO: 20. In another example, a forward primer has the nucleotide sequence as set forth in SEQ ID NO:, and a reverse primer has the nucleotide sequence as set forth in SEQ ID NO:. In another example, a forward primer has the nucleotide sequence as set forth in SEQ ID NO: 27, and a reverse primer has the nucleotide sequence as set forth in SEQ ID NO: 36. In another example, a forward primer has the nucleotide sequence as set forth in SEQ ID NO: 27, and a reverse primer has the nucleotide sequence as set forth in SEQ ID NO: 37. In another example, a forward primer has the nucleotide sequence as set forth in SEQ ID NO: 27, and a reverse primer has the nucleotide sequence as set forth in SEQ ID NO: 38. In another example, a forward primer has the nucleotide sequence as set forth in SEQ ID NO: 27, and a reverse primer has the nucleotide sequence as set forth in SEQ ID NO: 39. In another example, a forward primer has the nucleotide sequence as set forth in SEQ ID NO: 27, and a reverse primer has the nucleotide sequence as set forth in SEQ ID NO: 40. In another example, a forward primer has the nucleotide sequence as set forth in SEQ ID NO: 29, and a reverse primer has the nucleotide sequence as set forth in SEQ ID NO: 37. In another example, a forward primer has the nucleotide sequence as set forth in SEQ ID NO: 29, and a reverse primer has the nucleotide sequence as set forth in SEQ ID NO: 38. In another example, a forward primer has the nucleotide sequence as set forth in SEQ ID NO: 29, and a reverse primer has the nucleotide sequence as set forth in SEQ ID NO: 39. In another example, a forward primer has the nucleotide sequence as set forth in SEQ ID NO: 29, and a reverse primer has the nucleotide sequence as set forth in SEQ ID NO: 40. In another example, a forward primer has the nucleotide sequence as set forth in SEQ ID NO: 29, and a reverse primer has the nucleotide sequence as set forth in SEQ ID NO: 37. In another example, a forward primer has the nucleotide sequence as set forth in SEQ ID NO: 29, and a reverse primer has the nucleotide sequence as set forth in SEQ ID NO: 38. In another example, a forward primer has the nucleotide sequence as set forth in SEQ ID NO: 29, and a reverse primer has the nucleotide sequence as set forth in SEQ ID NO: 39. In another example, a forward primer has the nucleotide sequence as set forth in SEQ ID NO: 29, and a reverse primer has the nucleotide sequence as set forth in SEQ ID NO: 40. In another example, a forward primer has the nucleotide sequence as set forth in SEQ ID NO: 31, and a reverse primer has the nucleotide sequence as set forth in SEQ ID NO: 37. In another example, a forward primer has the nucleotide sequence as set forth in SEQ ID NO: 31, and a reverse primer has the nucleotide sequence as set forth in SEQ ID NO: 38. In another example, a forward primer has the nucleotide sequence as set forth in SEQ ID NO: 31, and a reverse primer has the nucleotide sequence as set forth in SEQ ID NO: 39. In another example, a forward primer has the nucleotide sequence as set forth in SEQ ID NO: 31, and a reverse primer has the nucleotide sequence as set forth in SEQ ID NO: 40. In another example, a forward primer has the nucleotide sequence as set forth in SEQ ID NO: 35, and a reverse primer has the nucleotide sequence as set forth in SEQ ID NO: 20. In another example, a forward primer has the nucleotide sequence as set forth in SEQ ID NO: 35, and a reverse primer has the nucleotide sequence as set forth in SEQ ID NO: 37. In another example, a forward primer has the nucleotide sequence as set forth in SEQ ID NO: 35, and a reverse primer has the nucleotide sequence as set forth in SEQ ID NO: 38. In another example, a forward primer has the nucleotide sequence as set forth in SEQ ID NO: 35, and a reverse primer has the nucleotide sequence as set forth in SEQ ID NO: 39. In another example, a forward primer has the nucleotide sequence as set forth in SEQ ID NO: 35, and a reverse primer has the nucleotide sequence as set forth in SEQ ID NO: 40.

In another example, the present invention provides a primer pair for providing highly-specific amplification of ilvC gene or mRNA sequences from M. tuberculosis or M. tuberculosis complex organisms, wherein said primer pairs are selected from the group consisting of:
(a) SEQ ID NO: 19 and SEQ ID NO: 20; and
(b) SEQ ID NO: 20 and a primer selected from SEQ ID NOs: 27, 29, 31 and 35.

In another example, the present invention provides a primer pair for providing highly-specific amplification of ilvC gene or mRNA sequences from M. tuberculosis or M. tuberculosis complex organisms, wherein said primer pairs are selected from the group consisting of:
(a) SEQ ID NO: 27 and a primer selected from SEQ ID NOs: 28, 30 and 32;
(b) SEQ ID NO: 29 and a primer selected from SEQ ID NOs: 30 and 32;
(c) SEQ ID NO: 31 and SEQ ID NO: 32.

In another example, the present invention provides a primer pair for providing highly-specific amplification of ilvC gene or mRNA sequences from M. tuberculosis or M. tuberculosis complex organisms, wherein said primer pairs are selected from the group consisting of:
(a) SEQ ID NO: 27 and a primer selected from SEQ ID NOs: 28 and 30; and
(b) SEQ ID NO: 29 and SEQ ID NO: 30.

In another example, the present invention provides a primer pair for providing highly-specific amplification of ilvC gene or mRNA sequences from M. tuberculosis or M. tuberculosis complex organisms, wherein said primer pairs are selected from the group consisting of:
(a) SEQ ID NO: 29 and a primer selected from SEQ ID NOs: 30 and 32; and
(b) SEQ ID NO: 31 and SEQ ID NO: 32.

In another example, the present invention provides a primer pair for providing highly-specific amplification of ilvC gene or mRNA sequences from M. tuberculosis or M. tuberculosis complex organisms, and optionally for use with a molecular beacon wherein said primer pairs are selected from the group consisting of:
(a) SEQ ID NO: 32 and SEQ ID NO: 38;
(b) SEQ ID NO: 32 and SEQ ID NO: 39;
(c) SEQ ID NO: 32 and SEQ ID NO: 40;
(d) SEQ ID NO: 35 and SEQ ID NO: 38;
(e) SEQ ID NO: 35 and SEQ ID NO: 39; and
(f) SEQ ID NO: 35 and SEQ ID NO: 40.

In another example, the present invention provides a primer pair for providing highly-specific amplification of ilvC gene or mRNA sequences from M. tuberculosis or M. tuberculosis complex organisms, and optionally for use with a molecular beacon wherein said primer pairs are selected from the group consisting of:
(a) SEQ ID NO: 32 and SEQ ID NO: 38;
(b) SEQ ID NO: 32 and SEQ ID NO: 40;
(c) SEQ ID NO: 35 and SEQ ID NO: 38; and
(d) SEQ ID NO: 35 and SEQ ID NO: 39.

As will be known to those skilled in the art, the length of a primer of the present invention may be varied by addition of one or more nucleotides to the 5′-end and/or 3′-end, or by deletion of one or more nucleotides from the 5′-end and/or 3′-end. In one example, the present invention clearly encompasses the use of one or more variant primers of a primer exemplified herein, wherein the variant primer comprises 1 or 2 or 3 or 4 or 5 additional nucleotides at the 5′-end and/or 3′-end relative to any one or more of SEQ ID NOs: 19, 20 or 27-32 subject to the proviso that any 3′-terminal addition to said SEQ ID NO(s) is contiguous with the nucleotide sequence of SEQ ID NO: 2 or a sequence complementary to SEQ ID NO: 2 immediately downstream of said SEQ ID NO(s) such that amplification of ilvC-specific sequence may occur using the variant primer. Alternatively, or in addition, a variant primer may also comprise a 5′-terminal addition to said SEQ ID NO(s) that is contiguous with the nucleotide sequence of SEQ ID NO: 2 or a sequence complementary to SEQ ID NO: 2 immediately upstream of said SEQ ID NO(s), however such a requirement for contiguity is generally not required for amplification of ilvC-specific sequence to occur using the variant primer. In another example, the present invention clearly encompasses the use of one or more variant primers of a primer exemplified herein, wherein the variant primer has 1 or 2 or 3 or 4 or 5 nucleotides removed from the 5′-end and/or 3′-end relative to any one or more of SEQ ID NOs: 19, 20 or 27-32 or 35-40.

### Nucleic Acid Sequence Based Amplification (NASBA)

Nucleic acid sequence-based amplification (NASBA) refers to any primer dependent, homogeneous, amplification process for the detection of RNA in the absence of thermal cycling e.g., under isothermal conditions such as at a constant temperature of about 41° C. NASBA can produce up to about 100-fold amplification of a specific RNA sequence, or more, and offers the unique possibility to amplify RNA in a background of genomic DNA. Detection of RNA can be used to monitor gene expression or cell viability or can be a prognostic indication for virus replication and production. Amplification of RNA beyond the detection limit is easily achieved when RNA targets are present at high copy number in a sample.

Briefly, an RNA template is provided to a reaction mixture under conditions such that a first primer attaches to its complementary site at the 3′ end of the template, a reverse transcriptase enzyme then synthesizes the complementary DNA and RNAse H enzyme degrades the RNA template of the RNA/DNA hybrids, and a second primer attaches to the 5′ end of the DNA strand to thereby permit T7 RNA polymerase to produces a complementary RNA strand which then acts as a template for a second cycle.

### Polymerase Chain Reaction (PCR)

In one example, the detection means may comprise a PCR amplification assay format. As used herein, the term “PCR” or “polymerase chain reaction” shall be taken to mean an amplification reaction employing multiple cycles of (i) denaturation of double-stranded nucleic acid such as a nucleic acid “template” to be amplified or a hybrid between a “template” and a complementary “primer”; (ii) annealing of a primer to its complementary sequence in the single-stranded “template”; and (iii) extension of the primer in the 5′- to 3′-direction by a polymerase activity e.g., an activity of a thermostable polymerase, such as, Taq, to thereby produce a double-stranded nucleic acid comprising a newly-synthesized strand complementary to the single-stranded template. By utilizing two primers capable of annealing to the complementary strands in the double-stranded template (i.e., to each denatured single-stranded template), multiple copies of the template are produced in each cycle, thereby amplifying the template.

Many formats of PCR are known in the art and are contemplated for use in any embodiment as described herein including, for example, one-armed (or single-primer) PCR, reverse-transcriptase mediated PCR (RT-PCR), nested PCR, touch-up and loop incorporated primers (TULIP) PCR, touch-down PCR, competitive PCR, rapid competitive PCR (RC-PCR), multiplex PCR, LAMP, RT-LAMP and LAMP-ELISA-hybridization. In an other example, RT-PCR amplification is performed in a reaction comprising amplification primers and a molecular beacon, wherein the molecular beacon comprises a sequence capable of hybridizing to a nucleic acid product of the amplification primers to thereby produce a signal e.g., a fluorescent signal.

Methods of PCR are known in the art and described, for example, in Dieffenbach (ed) and Dveksler (ed) (In: PCR Primer: A Laboratory Manual, Cold Spring Harbour Laboratories, NY, 1995). Generally, for PCR two non-complementary nucleic acid primers comprising at least about 8, more preferably, at least about 15 or 20 nucleotides are annealed to different strands of a template nucleic acid, and amplified nucleic acid of the template are amplified enzymatically using a polymerase, preferably, a thermostable DNA polymerase.

Reagents required for PCR are known in the art and include for example, one or more primers (described herein), a suitable polymerase, deoxynucleotides and/or ribonucleotides, a buffer. Suitable reagents are described for example, in Dieffenbach (ed) and Dveksler (ed) (In: PCR Primer: A Laboratory Manual, Cold Spring Harbour Laboratories, NY, 1995).

For example, a suitable polymerase for use in the method of the invention include, a DNA polymerase, a RNA polymerase, a reverse transcriptase, a T7 polymerase, a SP6 polymerase, a T3 polymerase, Sequenase™, a Klenow fragment, a Taq polymerase, a Taq polymerase derivative, a Taq polymerase variant, a Pfu polymerase, a Pfx polymerase, an AmpliTaq™ FS polymerase, a thermostable DNA polymerase with minimal or no 3′-5′ exonuclease activity, or an enzymatically active variant or fragment of any of the above polymerases. Preferably, a polymerase used in the method of the invention is a thermostable polymerase.

In one embodiment, a mixture of two or more polymerases is used. For example, the mixture of a Pfx or Pfu polymerase and a Taq polymerase has been previously shown to be useful for amplifying templates comprising a high GC content or for amplifying a large template.

Suitable commercial sources for a polymerase useful for the performance of the invention will be apparent to the skilled artisan and include, for example, Stratagene (La Jolla, Calif., USA), Promega (Madison, Wis., USA), Invitrogen (Carlsbad, Calif., USA), Applied Biosystems (Foster City, Calif., USA) and New England Biolabs (Beverly, Mass., USA).

The specific amplification of an ilvC nucleic acid of M. tb or M.tb complex in accordance with the invention may utilize about 25 or 30 or 35 or 40 or 45 or 50 amplification cycles, and more preferably more than 28 or 29 or 30 or 31 or 32 or 33 or 34 or 35 amplification cycles. Optionally, where a larger number of amplification cycles is performed, non-specific amplification products e.g., primer dimers and or M. avium nucleic acid, are excluded e.g., by resolving the amplification products and excluding products that do not comprise M.tb ilvC nucleic acid or M.tb complex ilvC nucleic acid. Standard means are employed to resolve specific and non-specific amplification products and exclude non-specific products e.g., by producing melt curves for the amplification products and selecting an amplification product having a melt curve that indicates a Tm of M. tb or M. tb complex nucleic acid product, and/or by performing gel electrophoresis to resolve the amplification products and selecting a nucleic acid fragment in the electophoretogram having a size/length that indicates a Tm of M. tb or M. tb complex nucleic acid product.

In another example, the specific amplification of an ilvC nucleic acid of M. tb or M.tb complex in accordance with the invention utilizes a lower number of amplification cycles e.g., to reduce non-specific amplification effects such as amplification of M. avium sequence for certain primer pairs described herein. For example, a standard amplification protocol may employ less than 30 cycles or less than 29 cycles or less than 28 cycles or less than 27 cycles or less than 26 cycles or less than 25 cycles. Alternatively, or in addition, at least about 12 cycles or at least about 13 cycles or at least about 14 cycles or at least about 15 cycles or at least about 16 cycles or at least about 17 cycles or at least about 18 cycles or at least about 19 cycles or at least about 20 cycles or at least about 21 cycles or at least about 22 cycles are employed. A particularly preferred cycle protocol employs about 17 or 18 or 19 or 20 or 21 or 22 or 23 or 24 or 25 or 26 or 27 amplification cycles, more preferably about 22 or 23 or 24 or 25 or 26 or 27 amplification cycles, even more preferably about 22 or 23 or 24 or 25 amplification cycles.

In another example, nucleic acid template at a preferred concentration herein is combined with a preferred primer pair referred to herein and amplification performed over about 17 to about 27 amplification cycles or for about 22 to about 25 amplification cycles. Conventional primer concentrations may be employed, and conveniently the exemplified primers are at about 50 nM concentration or about 100 nM× concentration or about 150 nM concentration or about 200 nM concentration or about 250 nM concentration.

### Reverse Transcription PCR (RT-PCR)

For RT-PCR, RNA is reverse transcribed using a reverse transcriptase (such as, for example, Moloney Murine Leukemia Virus reverse transcriptase) to produce cDNA. In this regard, the reverse transcription of the RNA is primed using, for example, a random primer (e.g., a hexa-nucleotide random primer) or oligo-dT (that binds to a poly-adenylation signal in mRNA). Alternatively, a site-specific primer is used to prime the reverse transcription. A sample is heated to ensure production of single stranded nucleic acid and then cooled to enable annealing of the primer. The sample is then incubated under conditions sufficient for reverse-transcription of the nucleic acid adjacent to an annealed primer by a reverse transcriptase. Following reverse transcription, the cDNA is used as a template nucleic acid for a PCR reaction, e.g., standard, PCR, or any other PCR as described herein, for example, it may be combined with LAMP (i.e. RT-LAMP). It will be apparent that any commercially available kit for producing cDNA is also contemplated by the method of the invention, e.g., using a cDNA synthesis kit (Marligen).

A person skilled in the art will understand that any RT-PCR may also comprise the use of an internal reference standard to quantitate target RNA. In this respect, when dealing with the highly purified intact RNA samples, intact total RNA can be estimated by OD values. However, crude RNA samples are also contemplated in the method of the invention for use in RT-PCR. In the case of crude RNA samples; intact RNA is smaller than the amount estimated by OD values due to DNA contaminants and/or degraded RNA. Therefore, OD values cannot be used for the comparison among crude RNA samples. Especially, when comparing the expression level of the target gene among samples, RNA amounts can be corrected to obtain precise comparison results. To assay the expression level of the target gene in cells precisely, RNA amounts applied to the assay can be corrected to the fixed amounts among the samples. In this respect, to correct the RNA amount applicable to the assay among the samples, by performing RT-PCR amplifying internal reference template such as housekeeping genes.

Suitable housekeeping genes for use in the method of the invention will be apparent to a person skilled in the art. In one example, it is preferred that the housekeeping gene is generally expressed at high levels such that the expression product is detectable and provides a suitable estimate of total intact RNA. For example, the housekeeping gene may be RNA from the host, e.g., β-actin. In another example, the housekeeping gene is an expression product of any one of the organisms of the M. tuberculosis complex, e.g., 16S rRNA. In another example, detection the use of primers that detect all 16S rRNA species are contemplated.

### One-Armed PCR

As the name suggests single primer PCR uses only one primer to amplify nucleic acid. Generally this technique comprises annealing a first primer to template nucleic acid at sufficiently low stringency to ensure that it anneals to multiple sites in the template. Following polymerase mediated replication, nucleic acid products are produced in those cases wherein the primer has annealed sufficiently closely to enable amplification. Using a second primer or set thereof, the amplified nucleic acid is amplified further.

### Nested PCR

Nested PCR is described in detail in, for example, Dieffenbach (ed) and Dveksler (ed) (In: PCR Primer: A Laboratory Manual, Cold Spring Harbour Laboratories, NY, 1995). Essentially a nested PCR reaction involves the use of two sets of primers that are specific to a sequence of interest. The first set of primers is used to amplify the nucleic acid template to a desired level. The second set of primers is designed to anneal to a region of the nucleic acid between the sites of annealing of the first primers and further amplify the nucleic acid template.

The nested PCR reaction may be performed with all primers in a single closed tube. Alternatively, the initial PCR is performed in one tube and the second PCR is performed in a separate closed tube.

### Touchdown PCR

Touchdown PCR is another modification of conventional PCR that also reduces nonspecific amplification. Touchdown PCR is described in detail in Don R H et al (Nucleic Acids Res. 1991 Jul. 25; 19(14):4008). Essentially, Touchdown PCR involves the use of an annealing temperature that is higher than the target optimum in early PCR cycles. The annealing temperature is decreased by 1° C. every cycle or every second cycle until a specified or ‘touchdown’ annealing temperature is reached. The touchdown temperature is then used for the remaining number of cycles. This allows for the enrichment of the correct product over any non-specific product.

### Touch-Up and Loop Incorporated Primer (TULIP)-PCR

TULIPS-PCR is described in detail, for example, in Ailenberg, M., et al. (BioTechniques 29(5), 1018-23 (2000)). TULIPS-PCR utilizes loop primers which are additional non-template 5′ sequence that set-anneals to the 3′ region and inhibits initiation of polymerisation. Upon heating of the reaction, the primers melt, initiating hot start. The reaction also uses touch-up pre-cycling with gradual elevation in annealing temperatures to ensure correct pairing. Further details on primer design and reaction conditions may be found online, for example, at Ailenberg and Silverman (Application Note, online reference: http://209.197.88.225/articles/abl/b0110ail.pdf).

### Competitive and Rapid Competitive PCR

Competitive PCR involves the coamplification of a target DNA or RNA sample with known amounts of a competitor DNA or RNA that shares most of the nucleotide sequence with the target; in this way, any predictable or unpredictable variable affecting PCR amplification has the same effect on both molecular species. Competitive PCR therefore permits the quantification of the absolute number of target molecules in comparison to the amount of competitor DNA. A person skilled in the art will understand that competitive PCR is a fast and accurate method for quantifying numbers of target molecules. Competitive PCR is described in detail, for example, in Celi et al, (Nucleic Acids Research 21; p. 1047 (1991)); Schneegberger et al, PCR Methods and Applications 4; p. 234-238 (1995)); and in Quantitative RT-PCR (Methods and Applications Book 3; Clontech Laboratories, Inc.).

In this method quantitation of target DNA or RNA can be relatively quantified compared with the initial amount of competitor. Initial amount of target DNA or RNA can be estimated by T/C* ratio.

*T: Amount of amplified product from target DNA or RNA
C: Amount of amplified product from competitor

Initial amount of target DNA or RNA will correspond to the competitor's amount, when T/C ratio=1.

Competitive PCR generally comprises the addition of serial dilutions of internal standard. A variation of competitive PCR, rapid competitive PCR (RC-PCR) may also be used in the method of the invention. RC-PCR is characterized by measuring relative gene expression at the mRNA level of two or more samples with a nonradioactive assay based on competitive PCR amplification between identical sequences of internal standard and target cDNA. Only a single reaction tube per sample is used in this technique. RC-PCR is described in detail, for example, in Jiang et al (Clinical Chemistry, 42(2): 227-231).

### Loop-Mediated Isothermal Amplification (LAMP)

The method of the present invention also contemplates the use of loop-mediated isothermal amplification (LAMP). LAMP works on the amplification of 6 distant regions in target DNA autocycled by Bst DNA polymerase in one tube. This reaction can produce extremely high amounts of target DNA and/or RNA fragment for detection at a constant temperature. LAMP is described in detail, for example, in Notomi et al (Nucleic Acids Res. 28: E63 (2000)). A person skilled in the art would also be able to use an online tool for designing LAMP primers using an online primer tool, for example the Netlaboratory support software program at http://primerexplorer.jp/e/index.html).

The ilvC Reaction Product or Amplicon

The one or more ilvC nucleic acids detected in the assay or the amplicon may comprise a sequence of at least about 20 or 30 contiguous nucleotides in length of SEQ ID NO: 2. Amplicons will generally comprise at least about 40 or 50 contiguous nucleotides in length of SEQ ID NO: 2, because they will include the sequences of amplification primers which are generally each at least about 12-15 nucleotides in length. For example, the ilvC nucleic acids detected in the assay or the amplicon may comprise a sequence of at least about 55 or at least about 60 or at least about 65 or at least about 70 or at least about 75 or at least about 80 or at least about 85 or at least about 90 or at least about 95 or at least about 100 or at least about 105 or at least about 110 or at least about 115 or at least about 120 or at least about 125 or at least about 130 or at least about 135 or at least about 140 or at least about 145 or at least about 150 or at least about 155 or at least about 160 contiguous nucleotides in length of SEQ ID NO: 2. Exemplary amplicons described herein are in the range of about 75 to abut 135 nucleotides in length Longer ilvC nucleic acids may be detected, or longer amplicons produced, in the assay of the present invention, e.g., about 900 contiguous nucleotides of SEQ ID NO 2 or the entire sequence of SEQ ID NO: 2.

### Detection and Quantitation of Amplified Nucleic Acid

It will be apparent to the skilled artisan that any method of detecting an amplified nucleic acid known in the art is contemplated in the method of the invention. A variety of methods known to the skilled artisan may be employed, including the use of probes such as TaqMan probes, SYBR green I or II-labelled primers, molecular beacons, or scorpion probes.

In one example, a TaqMan Probe is employed. TaqMan probes depend on the 5′-nuclease activity of the DNA polymerase used for PCR to hydrolyze an oligonucleotide that is hybridized to the target amplicon. TaqMan probes are oligonucleotides that have a fluorescent reporter dye attached to the 5′-end and a quencher moeity coupled to the 3′-end. These probes are designed to hybridize to an internal region of a PCR product. In the unhybridized state, the proximity of the fluor and the quench molecules prevents the detection of fluorescent signal from the probe. During PCR, when the polymerase replicates a template on which a TaqMan probe is bound, the 5′-nuclease activity of the polymerase cleaves the probe. This decouples the fluorescent and quenching dyes and FRET no longer occurs. Thus, fluorescence increases in each cycle, proportional to the amount of probe cleavage. Well-designed TaqMan probes may require little optimization. TaqMan probes are also used for multiplex assays described herein e.g., by designing each probe with a spectrally unique fluor/quench pair.

In another example, a Molecular Beacon is employed. Molecular Beacons use FRET to detect and quantitate a PCR product i.e., amplicon via a fluor coupled to the 5′-end and a quench attached to the 3′-end of an oligonucleotide substrate. Unlike TaqMan probes, Molecular Beacons remain intact during the amplification reaction, and rebind to target in every cycle for signal measurement. Molecular Beacons form a stem-loop structure when free in solution and the close proximity of the fluor and quench molecules prevents the probe from fluorescing. When a Molecular Beacon hybridizes to a target, the fluorescent dye and quencher are separated, and the fluorescent dye emits light upon irradiation. Molecular Beacons can be used for multiplex assays described herein by using spectrally separated fluor/quench moieties on each probe.

In another example, a Scorpion probe is employed. Scorpion probes provide for sequence-specific priming and amplicon detection using a single oligonucleotide. A suitable Scorpion probe maintains a stem-loop configuration in the unhybridized state and, by virtue of having a fluorophore attached to the 5′-end and a quencher moiety attached to the 3′-end signal is quenched in the unhybridized state. The 3′-portion of the stem comprises a sequence that is complementary to the extension product of the primer and that is linked to the 5′-end of a specific primer via a non-amplifiable monomer. After extension from a Scorpion primer, the specific probe sequence hybridizes to a complementary sequence in the extended amplicon thereby opening the hairpin loop to prevent quenching of fluorescence and providing for a signal.

In another example, SYBR Green is employed in an amplification primer or probe. SYBR Green provides a simple and economic format for detecting and quantitating PCR products in real-time reactions. SYBR Green binds to double-stranded DNA e.g., an amplicon produced in RT-PCR or standard PCR, to thereby emit light on excitation. Accordingly, the level of fluorescence produced is proportional to the amount of amplicon produced. SYBR Green works well and with low non-specific background signal being generated.

In accordance with these examples, a probe e.g., a molecular beacon or TaqMan probe or Scorpion probe, will comprise a sequence contained within a strand of an amplicon produced by the amplification primers. Any primer combination referred to herein may be employed in this example of the invention subject to the proviso that the primer combination provides for an amplicon of sufficient length for a probe e.g., a molecular beacon or TaqMan probe or Scorpion probe, to hybridize to it. An exemplary probe e.g., a molecular beacon or TaqMan probe or Scorpion probe, will comprise the sequence set forth in SEQ ID NO: 33 or 34. Exemplary primer combinations for use with a probe comprising SEQ ID NO: 33 or 34 are selected from the group consisting of (a) SEQ ID NO: 32 and SEQ ID NO: 38;
(b) SEQ ID NO: 32 and SEQ ID NO: 39;
(c) SEQ ID NO: 32 and SEQ ID NO: 40;
(d) SEQ ID NO: 35 and SEQ ID NO: 38;
(e) SEQ ID NO: 35 and SEQ ID NO: 39; and
(f) SEQ ID NO: 35 and SEQ ID NO: 40.

Variants of these primers may also be employed e.g., variants comprising 5′-end and/or 3′-end additions or deletions as described herein above.

Exemplary primer combinations for use with a probe e.g., a molecular beacon or TaqMan probe or Scorpion probe, comprising SEQ ID NO: 33 or 34 are also selected from the group consisting of:
(a) SEQ ID NO: 32 and SEQ ID NO: 38;
(b) SEQ ID NO: 32 and SEQ ID NO: 40;
(c) SEQ ID NO: 35 and SEQ ID NO: 38; and
(d) SEQ ID NO: 35 and SEQ ID NO: 39.

Variants of these primers may also be employed e.g., variants comprising 5′-end and/or 3′-end additions or deletions as described herein above.

In another example, amplified nucleic acid(s) using the method of the invention is/are separated using gel electrophoresis. The separated nucleic acid(s) are then detected using a detectable marker that selectively binds nucleic acid, such as, for example, ethidium bromide, 4′-6-diamidino-2-phenylinodole (DAPI), methylene blue or SYBR® green I or II (available from Sigma Aldrich). Suitable methods for detection of a nucleic acid using gel electrophoresis are known in the art and described, for example, in Ausubel et al (In: Current Protocols in Molecular Biology. Wiley Interscience, ISBN 047 150338, 1987) and Sambrook et al (In: Molecular Cloning: Molecular Cloning: A Laboratory Manual, Cold Spring Harbor Laboratories, New York, Third Edition 2001). For example, the nucleic acid is separated using one dimensional agarose, agaorse-acrylamide or polyacrylamide gel electrophoresis. Such separation techniques separate nucleic acids on the basis of molecular weight.

In another example, amplified nucleic acid(s) is separated using two dimensional electrophoresis and detected using a detectable marker (e.g., as described supra). Two dimensional agarose gel electrophoresis is adapted from the procedure by Bell and Byers Anal. Biochem. 130:527, 1983. The first dimension gel is run at low voltage in low percentage agarose to separate DNA molecules in proportion to their mass. The second dimension is run at high voltage in a gel of higher agarose concentration in the presence of ethidium bromide so that the mobility of a non-linear molecule is drastically influenced by its shape.

In another example, an amplification product is characterized or isolated using capillary electrophoresis. Capillary electrophoresis is reviewed in, for example, Heller, Electrophoresis 22:629-43, 2001; Dovichi et al., Methods Mol Biol 167:225-39, 2001; Mitchelson, Methods Mol Biol 162:3-26, 2001; or Dolnik, J Biochem Biophys Methods 41:103-19, 1999. Capillary electrophoresis uses high voltage to separate molecules according to their size and charge. A voltage gradient is produced in a column (i.e. a capillary) and this gradient drives molecules of different sizes and charges through the tube at different rates.

In another example, an amplification product is identified and/or isolated using chromatography. For example, ion pair-reversed phase HPLC has been shown to be useful for isolating a PCR product (Shaw-Bruha and Lamb, Biotechniques. 28:794-7, 2000.

The present invention also contemplates an automated or semi-automated method for detection of a nucleic acid of the invention. Automated systems are available from, for example, Applied Biosystems.

In another example, the amplified nucleic acid is detected using, for example, mass spectrometry (e.g., MALDI-TOF). For example, a sample comprising nucleic acid amplified using the method of the invention is incorporated into a matrix, such as for example 3-hydroxypropionic acid, α-cyano-4-hydroxycinnamic acid, 3,5 dimethoxy-4-hydroxycinnamic acid (Sinapinic acid) or 2,5 dihydroxybenzoic acid (Gentisic acid). The sample and matrix are then spotted onto a metal plate and subjected to irradiation by a laser, promoting the formation of molecular ions. The mass of the produced molecular ion is analyzed by its time of flight (TOF), essentially as described by Yates, J. Mass Spectrom. 33, 1-19, 1998 and references cited therein. A time of flight instrument measures the mass to charge ratio (m/z) ratio of an ion by determining the time required for it to traverse the length of a flight tube. Optionally, such a TOF mass analyzer includes an ion mirror at one end of the flight tube that reflects said ion back through the flight tube to a detector. Accordingly, an ion mirror serves to increase the length of a flight tube, increasing the accuracy of this form of analysis. By determining the time of flight of the ion, the molecular weight of an amplified nucleic acid is determined.

It will be apparent that any method of quantitation of the above detection methods is contemplated, e.g. by standard densitometry techniques.

In a preferred embodiment, any known method of quantitative PCR is employed, such as for example, real-time PCR. Real-time PCR is based on any PCR reaction, which is used to amplify and simultaneously quantify a targeted DNA molecule. It enables both detection and quantification (as absolute number of copies or relative amount when normalized to DNA input or additional normalizing genes) of a specific sequence in a DNA sample. The procedure follows the general principle of polymerase chain reaction; its key feature is that the amplified DNA is quantified as it accumulates in the reaction in real time after each amplification cycle. Two common methods of quantification are the use of fluorescent dyes that intercalate with double-stranded DNA, and modified DNA oligonucleotide probes that fluoresce when hybridized with a complementary DNA. Real-time PCR is particularly, useful when combined with RT-PCR to quantify mRNA transcripts. It will be apparent that any known method of real-time PCR and/or commercially available platforms may be used in the method of the invention, for example, as described in Whelan et al (Journal of Immunological Methods, 278: 261-269). As exemplified herein, the Rotor-Gene 3000 system (Corbett) has been used by the inventors according to the manufacturers protocols.

In another example, amplified nucleic acid may be detected using a solid phase detection system, e.g., ELISA. It is understood that any PCR assay format described herein is adaptable to a PCR-ELISA format. This is also particularly useful with a LAMP PCR format. In this example, NucleoLink tubes (Nalge Nunc International) may be employed. These tubes have an activated heat stable polymer that has a secondary amino group covalently grafted onto ist surface. Conditions for PCR-ELISA formats have been described in detail, for example, Wilson et al (Journal of Microbiological Methods, 51(2): 163-170 (2002)).

### Multiplex Assay Formats

It is within the scope of the present invention to include a multi-analyte NAA test in this assay format, wherein multiple nucleic acids are detected in addition to the ilvC nucleic acids. Other nucleic acids encoded from genes of other proteins derived from any one of the proteins expressed by one or more Mycobacteria of the M. tuberculosis complex are used to confirm a diagnosis obtained using ilvC-NAA.

Without limitation to a specific assay format, the method of the present invention also contemplates the use of multiplex NASBA or multiplex PCR. Multiplex methods amplifies multiple RNA or DNA targets using more than one pair of primers in one reaction tube.

A person skilled in the art will understand that quantitative multiplex reactions may require optimization to determine the primer concentration, template concentration, cycling condition and buffer components. Conditions and details for performing, and optimizing multiplex platforms are known see e.g., Garcia-Garcia et al (Hum Mutat. 27(8):822-8 (2006)); Tettelin et al, Genomics 62, 500-7 (1991)); Wittwer et al (Methods. 25(4):430-42 (2001)); Markoulatos et al (J Clin Lab Anal. 17(4):108-12 (2003)). Further details may also be found online, for example, at http://biowww.net/detail-416.html. A person skilled in the art would also be able to use an online tool for designing multiplex primers, for example as found at http://genomics14.bu.edu:8080/MuPlex/MuPlex.html developed by Rachlin et al, (muPlex: A Multi-Objective Approach to Multiplex PCR Assay Design. Nucleic Acids Research. 33 (Web Server Issue):W544-W547 (2005).

As will be apparent to those skilled in the art a diagnostic or prognostic assay described herein may be a multiplexed assay. As used herein the term “multiplex”, shall be understood not only to mean the detection of two or more diagnostic or prognostic markers in a single sample simultaneously, but also to encompass consecutive detection of two or more diagnostic or prognostic markers in a single sample, simultaneous detection of two or more diagnostic or prognostic markers in distinct but matched samples, and consecutive detection of two or more diagnostic or prognostic markers in distinct but matched samples. As used herein the term “matched samples” shall be understood to mean two or more samples derived from the same initial biological sample, or two or more biological samples isolated at the same point in time.

In one example, the primers are about 100% homologous to an ilvC-nucleic acid across two or more such nucleic acids of the M.tb. complex. In one example, the primers are about 99% homologous to an ilvC-nucleic acid across two or more such nucleic acids of the M.tb. complex. In one example, the primers are about 98% homologous to an ilvC-nucleic acid across two or more such nucleic acids of the M.tb. complex. In one example, the primers are about 97% homologous to an ilvC-nucleic acid across two or more such nucleic acids of the M.tb. complex. In one example, the primers are about 96% homologous to an ilvC-nucleic acid across two or more such nucleic acids of the M.tb. complex. In one example, the primers are about 95% homologous to an ilvC-nucleic acid across two or more such nucleic acids of the M.tb. complex. In one example, the primers are about 94% homologous to an ilvC-nucleic acid across two or more such nucleic acids of the M.tb. complex. In one example, the primers are about 93% homologous to an ilvC-nucleic acid across two or more such nucleic acids of the M.tb. complex. In one example, the primers are about 92% homologous to an ilvC-nucleic acid across two or more such nucleic acids of the M.tb. complex. In one example, the primers are about 91% homologous to an ilvC-nucleic acid across two or more such nucleic acids of the M.tb. complex. In one example, the primers are about 90% homologous to an ilvC-nucleic acid across two or more such nucleic acids of the M.tb. complex.

For example, the other M. tuberculosis complex-derived protein is selected from the group consisting of BSX protein (UnitProtKB/TrEMBL Accession No. A5TZK2; SEQ ID NO: 3), ribosomal protein S9 (UniProtKB/Swiss-Prot Accession No. A5U8B8; SEQ ID NO: 5), protein Rv1265 (UniProtKB/Swiss-Prot Accession No. P64789; SEQ ID NO: 7), elongation factor-Tu (EF-Tu) protein (UniProtKB/Swiss-Prot Accession No. A5U071; SEQ ID NO: 9), P5CR protein (UniProtKB/Swiss-Prot Accession No. Q11141; SEQ ID NO: 11), TetR-like protein (UnitProtKB/TrEMBL Accession No. A1QW92; SEQ ID NO: 13) glutamine synthase (GS) protein (UnitProtKB/TrEMBL Accession No. 033342; SEQ ID NO: 15) and/or the nucleic acid 16S rRNA (SEQ ID NO:27). It will be apparent to a skilled artisan that any 16s rRNA sequence that is expressed by one or a plurality of bacteria of the M. tuberculosis complex e.g., M. tuberculosis and/or M. bovis and/or M. africanum and/or M. canetti and/or M. microti can be readily obtained from available curated sequence databases, and as described herein. It is to be understood in this context that the stated proteins and encoding sequences include homologs of the exemplified proteins and encoding sequences exemplified by way of the Sequence Listing, wherein said homologs are expressed by one or a plurality of bacteria of the M. tuberculosis complex e.g., M. tuberculosis and/or M. bovis and/or M. africanum and/or M. canetti and/or M. microti.

The skilled artisan will be aware that UniProtKB/Swiss-Prot is a curated protein sequence database of the Swiss Institute of Bioinformatics providing data on protein function, domain structure, post-translational modifications, and variants; and that UniProtKB/TrEMBL is a computer-annotated supplement of Swiss-Prot that contains translations of EMBL nucleotide sequence entries not yet integrated in Swiss-Prot. Access to UniProtKB/Swiss-Prot and UniProtKB/TrEMBL data can be obtained readily e.g., via the ExPASy (Expert Protein Analysis System) proteomics server of the Swiss Institute of Bioinformatics.

Assays for one or more secondary analytes e.g., nucleic acids encoding BSX, S9, EF-Tu, P5CR, TetR-like protein, glutamine synthetase and or 16s rRNA, are conveniently performed in the same manner as described herein for detecting ilvC nucleic acids (ilvC-NAA) in clinical samples. Accordingly, a person skilled in the art will understand that in the detection of these secondary analytes the template is as defined herein for ilvC nucleic acids, except it is encoded by the gene of the protein represented by the secondary analyte. The assays may be performed simultaneously or at different times, and using the same or different patient samples.

In one example, the template is a nucleic acid encoding the BSX protein as set forth in SEQ ID NO: 3. In another example the template is a nucleic acid encoding a protein that is at least 80% homologous to the BSX protein as set forth in SEQ ID NO: 3. In another example the template is a nucleic acid encoding a protein that is between 80% to 100% homologous to the BSX protein as set forth in SEQ ID NO: 3. In another example the template is a nucleic acid encoding a protein that is between 85% to 100% homologous to the BSX protein as set forth in SEQ ID NO: 3. In another example the template is a nucleic acid encoding a protein that is between 90% to 100% homologous to the BSX protein as set forth in SEQ ID NO: 3. In another example the template is a nucleic acid encoding a protein that is between 95% to 100% homologous to the BSX protein as set forth in SEQ ID NO: 3.

In another example, the template is a nucleotide coding sequence as set forth in SEQ ID NO: 4. In another example the template is a nucleotide coding sequence that is at least 80% homologous to the coding sequence as set forth in SEQ ID NO: 4. In another example the template is a nucleotide coding sequence that is at least 80% to 100% homologous to the coding sequence as set forth in SEQ ID NO: 4. In another example the template is a nucleotide coding sequence that is at least 85% to 100% homologous to the coding sequence as set forth in SEQ ID NO: 4. In another example the template is a nucleotide coding sequence that is at least 90% to 100% homologous to the coding sequence as set forth in SEQ ID NO: 4. In another example the template is a nucleotide coding sequence that is at least 95% to 100% homologous to the coding sequence as set forth in SEQ ID NO: 4. In another example the template is a nucleotide coding sequence that is at least 85% to 100% homologous to the coding sequence as set forth in SEQ ID NO: 4.

In one example, the template is a nucleic acid encoding the S9 protein as set forth in SEQ ID NO: 5. In another example the template is a nucleic acid encoding a protein that is at least 80% homologous to the S9 protein as set forth in SEQ ID NO: 5. In another example the template is a nucleic acid encoding a protein that is between 80% to 100% homologous to the S9 protein as set forth in SEQ ID NO: 5. In another example the template is a nucleic acid encoding a protein that is between 85% to 100% homologous to the S9 protein as set forth in SEQ ID NO: 5. In another example the template is a nucleic acid encoding a protein that is between 90% to 100% homologous to the S9 protein as set forth in SEQ ID NO: 5. In another example the template is a nucleic acid encoding a protein that is between 95% to 100% homologous to the S9 protein as set forth in SEQ ID NO: 5.

In another example, the template is a nucleotide coding sequence as set forth in SEQ ID NO: 6. In another example the template is a nucleotide coding sequence that is at least 80% homologous to the coding sequence as set forth in SEQ ID NO: 6. In another example the template is a nucleotide coding sequence that is at least 80% to 100% homologous to the coding sequence as set forth in SEQ ID NO: 6. In another example the template is a nucleotide coding sequence that is at least 85% to 100% homologous to the coding sequence as set forth in SEQ ID NO: 6. In another example the template is a nucleotide coding sequence that is at least 90% to 100% homologous to the coding sequence as set forth in SEQ ID NO: 6. In another example the template is a nucleotide coding sequence that is at least 95% to 100% homologous to the coding sequence as set forth in SEQ ID NO: 6. In another example the template is a nucleotide coding sequence that is at least 85% to 100% homologous to the coding sequence as set forth in SEQ ID NO: 6.

In one example, the template is a nucleic acid encoding the Rv1265 protein as set forth in SEQ ID NO: 7. In another example the template is a nucleic acid encoding a protein that is at least 80% homologous to the Rv1265 protein as set forth in SEQ ID NO: 7. In another example the template is a nucleic acid encoding a protein that is between 80% to 100% homologous to the Rv1265 protein as set forth in SEQ ID NO: 7. In another example the template is a nucleic acid encoding a protein that is between 85% to 100% homologous to the Rv1265 protein as set forth in SEQ ID NO: 7. In another example the template is a nucleic acid encoding a protein that is between 90% to 100% homologous to the Rv1265 protein as set forth in SEQ ID NO: 7. In another example the template is a nucleic acid encoding a protein that is between 95% to 100% homologous to the Rv1265 protein as set forth in SEQ ID NO: 7.

In another example, the template is a nucleotide coding sequence as set forth in SEQ ID NO: 8. In another example the template is a nucleotide coding sequence that is at least 80% homologous to the coding sequence as set forth in SEQ ID NO: 8. In another example the template is a nucleotide coding sequence that is at least 80% to 100% homologous to the coding sequence as set forth in SEQ ID NO: 8. In another example the template is a nucleotide coding sequence that is at least 85% to 100% homologous to the coding sequence as set forth in SEQ ID NO: 8. In another example the template is a nucleotide coding sequence that is at least 90% to 100% homologous to the coding sequence as set forth in SEQ ID NO: 8. In another example the template is a nucleotide coding sequence that is at least 95% to 100% homologous to the coding sequence as set forth in SEQ ID NO: 8. In another example the template is a nucleotide coding sequence that is at least 85% to 100% homologous to the coding sequence as set forth in SEQ ID NO: 8.

In one example, the template is a nucleic acid encoding the EF-Tu protein as set forth in SEQ ID NO: 9. In another example the template is a nucleic acid encoding a protein that is at least 80% homologous to the EF-Tu protein as set forth in SEQ ID NO: 9. In another example the template is a nucleic acid encoding a protein that is between 80% to 100% homologous to the EF-Tu protein as set forth in SEQ ID NO: 9. In another example the template is a nucleic acid encoding a protein that is between 85% to 100% homologous to the EF-Tu protein as set forth in SEQ ID NO: 9. In another example the template is a nucleic acid encoding a protein that is between 90% to 100% homologous to the EF-Tu protein as set forth in SEQ ID NO: 9. In another example the template is a nucleic acid encoding a protein that is between 95% to 100% homologous to the EF-Tu protein as set forth in SEQ ID NO: 9.

In another example, the template is a nucleotide coding sequence as set forth in SEQ ID NO: 10. In another example the template is a nucleotide coding sequence that is at least 80% homologous to the coding sequence as set forth in SEQ ID NO: 10. In another example the template is a nucleotide coding sequence that is at least 80% to 100% homologous to the coding sequence as set forth in SEQ ID NO: 10. In another example the template is a nucleotide coding sequence that is at least 85% to 100% homologous to the coding sequence as set forth in SEQ ID NO: 10. In another example the template is a nucleotide coding sequence that is at least 90% to 100% homologous to the coding sequence as set forth in SEQ ID NO: 10. In another example the template is a nucleotide coding sequence that is at least 95% to 100% homologous to the coding sequence as set forth in SEQ ID NO: 10. In another example the template is a nucleotide coding sequence that is at least 85% to 100% homologous to the coding sequence as set forth in SEQ ID NO: 10.

In one example, the template is a nucleic acid encoding the P5CR protein as set forth in SEQ ID NO: 11. In another example the template is a nucleic acid encoding a protein that is at least 80% homologous to the P5CR protein as set forth in SEQ ID NO: 11. In another example the template is a nucleic acid encoding a protein that is between 80% to 100% homologous to the P5CR protein as set forth in SEQ ID NO: 11. In another example the template is a nucleic acid encoding a protein that is between 85% to 100% homologous to the P5CR protein as set forth in SEQ ID NO: 11. In another example the template is a nucleic acid encoding a protein that is between 90% to 100% homologous to the P5CR protein as set forth in SEQ ID NO: 11. In another example the template is a nucleic acid encoding a protein that is between 95% to 100% homologous to the P5CR protein as set forth in SEQ ID NO: 11.

In another example, the template is a nucleotide coding sequence as set forth in SEQ ID NO: 12. In another example the template is a nucleotide coding sequence that is at least 80% homologous to the coding sequence as set forth in SEQ ID NO: 12. In another example the template is a nucleotide coding sequence that is at least 80% to 100% homologous to the coding sequence as set forth in SEQ ID NO: 12. In another example the template is a nucleotide coding sequence that is at least 85% to 100% homologous to the coding sequence as set forth in SEQ ID NO: 12. In another example the template is a nucleotide coding sequence that is at least 90% to 100% homologous to the coding sequence as set forth in SEQ ID NO: 12. In another example the template is a nucleotide coding sequence that is at least 95% to 100% homologous to the coding sequence as set forth in SEQ ID NO: 12. In another example the template is a nucleotide coding sequence that is at least 85% to 100% homologous to the coding sequence as set forth in SEQ ID NO: 12.

In one example, the template is a nucleic acid encoding the TetR-like protein as set forth in SEQ ID NO: 13. In another example the template is a nucleic acid encoding a protein that is at least 80% homologous to the TetR-like protein as set forth in SEQ ID NO: 13. In another example the template is a nucleic acid encoding a protein that is between 80% to 100% homologous to the TetR-like protein as set forth in SEQ ID NO: 13. In another example the template is a nucleic acid encoding a protein that is between 85% to 100% homologous to the TetR-like protein as set forth in SEQ ID NO: 13. In another example the template is a nucleic acid encoding a protein that is between 90% to 100% homologous to the TetR-like protein as set forth in SEQ ID NO: 13. In another example the template is a nucleic acid encoding a protein that is between 95% to 100% homologous to the TetR-like protein as set forth in SEQ ID NO: 13.

In another example, the template is a nucleotide coding sequence as set forth in SEQ ID NO: 14. In another example the template is a nucleotide coding sequence that is at least 80% homologous to the coding sequence as set forth in SEQ ID NO: 14. In another example the template is a nucleotide coding sequence that is at least 80% to 100% homologous to the coding sequence as set forth in SEQ ID NO: 14. In another example the template is a nucleotide coding sequence that is at least 85% to 100% homologous to the coding sequence as set forth in SEQ ID NO: 14. In another example the template is a nucleotide coding sequence that is at least 90% to 100% homologous to the coding sequence as set forth in SEQ ID NO: 14. In another example the template is a nucleotide coding sequence that is at least 95% to 100% homologous to the coding sequence as set forth in SEQ ID NO: 14. In another example the template is a nucleotide coding sequence that is at least 85% to 100% homologous to the coding sequence as set forth in SEQ ID NO: 14.

In one example, the template is a nucleic acid encoding the GS protein as set forth in SEQ ID NO: 15. In another example the template is a nucleic acid encoding a protein that is at least 80% homologous to the GS protein as set forth in SEQ ID NO: 15. In another example the template is a nucleic acid encoding a protein that is between 80% to 100% homologous to the GS protein as set forth in SEQ ID NO: 15. In another example the template is a nucleic acid encoding a protein that is between 85% to 100% homologous to the GS protein as set forth in SEQ ID NO: 15. In another example the template is a nucleic acid encoding a protein that is between 90% to 100% homologous to the GS protein as set forth in SEQ ID NO: 15. In another example the template is a nucleic acid encoding a protein that is between 95% to 100% homologous to the GS protein as set forth in SEQ ID NO: 15.

In one example, the template is a nucleic acid encoding a 16S rRNA of one or more bacteria of the M. Tuberculosis complex. In another example the template is a nucleic acid encoding a 16S rRNA that is at least 80% homologous to a nucleic acid encoding a 16s RNA of one or more bacteria of the M. Tuberculosis complex. In another example the template is a nucleic acid encoding 16S rRNA that is between 80% to 100% homologous to a nucleic acid encoding a 16s RNA of one or more bacteria of the M. Tuberculosis complex. In another example the template is a nucleic acid encoding a 16S rRNA that is between 85% to 100% homologous to a nucleic acid encoding a 16s RNA of one or more bacteria of the M. Tuberculosis complex. In another example the template is a nucleic acid encoding rRNA that is between 90% to 100% homologous to a nucleic acid encoding a 16s RNA of one or more bacteria of the M. Tuberculosis complex. In another example the template is a nucleic acid encoding a protein that is between 95% to 100% homologous to a nucleic acid encoding a 16s RNA of one or more bacteria of the M. Tuberculosis complex.

In another example, the template is a nucleotide coding sequence as set forth in SEQ ID NO: 16. In another example the template is a nucleotide coding sequence that is at least 80% homologous to the coding sequence as set forth in SEQ ID NO: 16. In another example the template is a nucleotide coding sequence that is at least 80% to 100% homologous to the coding sequence as set forth in SEQ ID NO: 16. In another example the template is a nucleotide coding sequence that is at least 85% to 100% homologous to the coding sequence as set forth in SEQ ID NO: 16. In another example the template is a nucleotide coding sequence that is at least 90% to 100% homologous to the coding sequence as set forth in SEQ ID NO: 16. In another example the template is a nucleotide coding sequence that is at least 95% to 100% homologous to the coding sequence as set forth in SEQ ID NO: 16. In another example the template is a nucleotide coding sequence that is at least 85% to 100% homologous to the coding sequence as set forth in SEQ ID NO: 16.

In one example, the template is a nucleic acid encoding a 16S rRNA of one or more bacteria of the M. Tuberculosis complex. In another example the template is a nucleic acid encoding a 16S rRNA that is at least 80% homologous to a nucleic acid encoding a 16s RNA of one or more bacteria of the M. Tuberculosis complex. In another example the template is a nucleic acid encoding 16S rRNA that is between 80% to 100% homologous to a nucleic acid encoding a 16s RNA of one or more bacteria of the M. Tuberculosis complex. In another example the template is a nucleic acid encoding a 16S rRNA that is between 85% to 100% homologous to a nucleic acid encoding a 16s RNA of one or more bacteria of the M. Tuberculosis complex. In another example the template is a nucleic acid encoding rRNA that is between 90% to 100% homologous to a nucleic acid encoding a 16s RNA of one or more bacteria of the M. Tuberculosis complex. In another example the template is a nucleic acid encoding a protein that is between 95% to 100% homologous to a nucleic acid encoding a 16s RNA of one or more bacteria of the M. Tuberculosis complex.

It is to be understood in this context that the stated proteins and/or rRNA and sequences encoding said proteins and/or rRNA include homologs of the exemplified proteins and/or rRNA and encoding sequences exemplified by way of the Sequence Listing, wherein said homologs are expressed by one or a plurality of bacteria of the M. tuberculosis complex e.g., M. tuberculosis and/or M. bovis and/or M. africanum and/or M. canetti and/or M. microti.

It will be understood by the skilled artisan any one or more primers for amplifying the one or more secondary analytes e.g., nucleic acids encoding BSX, S9, EF-Tu, P5CR, TetR-like protein, glutamine synthetase and/or 16s rRNA, are designed as described herein for the ilvC nucleic acids such that the one or more primer(s) comprises a sequence having at least about 80% identity overall to a strand of each corresponding template nucleic acid. More preferably, the degree of sequence identity is at least about 85% or 90% or 95% or 98% or 99%. For example, the primer or a region of a primer may comprise a sequence having at least about 80% identity to a region of a strand of a template of interest.

In one example, for amplifying nucleic acids encoding BSX, the forward primer has the nucleotide sequence as set forth in SEQ ID NO: 21, and the reverse primer has the nucleotide sequence as set forth in SEQ ID NO: 22.

In another example, for amplifying nucleic acids encoding Rv1265, the forward primer has the nucleotide sequence as set forth in SEQ ID NO: 23, and the reverse primer has the nucleotide sequence as set forth in SEQ ID NO: 24.

In another example, for amplifying nucleic acids encoding S9, the forward primer has the nucleotide sequence as set forth in SEQ ID NO: 25, and the reverse primer has the nucleotide sequence as set forth in SEQ ID NO: 26.

Real-time Reporters for Multiplex assays may also be employed in multiplex assay formats e.g., TaqMan probes, Molecular Beacons or Scorpion probes, to thereby allow multiple RNA or DNA species to be measured in the same sample, subject to the proviso that fluorescent dyes with different emission spectra are attached to different probes. Multiplex reactions also provides for internal controls to be co-amplified in single-tube, homogeneous assays.

### NAA-Antigen Combined Assay Formats

It will be understood that any one of the embodiments described herein supra may be combined with an antigen-based test for detecting KARI protein or a fragment thereof e.g., as described in AU 2008902611, such as to confirm the diagnosis and/or prognosis. In particular, one advantage of detecting M. tuberculosis antigen, as opposed to an antibody-based assay is that severely immune-compromised patients may not produce antibody at detectable levels, and the level of the antibody in any patient does not reflect bacilli burden. On the other hand antigen levels should reflect bacilli burden and, being a product of the bacilli, are a direct method of detecting its presence.

In one example, the ilvC-NAA diagnostic and prognostic assays as described according to any embodiment herein are combined with an antigen-based assay to provide a combined NAA-antigen based assay, wherein said antigen-based assay comprises detecting in a biological sample from said subject a KARI protein, or a fragment thereof. Combined assays are performed simultaneously or in any order e.g., NAA followed by antigen-based detection or antigen-based detection followed by NAA. For example, the biological sample is contacted with an isolated ligand, e.g., a small molecule, peptide, antibody, or immune reactive fragment of an antibody, that binds specifically to an immunogenic KARI protein of Mycobacterium tuberculosis or other organism of the M. tuberculosis complex, an immunogenic KARI peptide or immunogenic KARI fragment or epitope thereof. Preferred ligands are peptides or antibodies. Preferred antibodies include, for example, a monoclonal or polyclonal antibody preparation. This extends to any isolated antibody-producing cell or antibody-producing cell population, e.g., a hybridoma or plasmacytoma producing antibodies that bind to a KARI protein or immunogenic fragment of a KARI protein or other immunogenic peptide comprising a sequence derived from the sequence of a KARI protein. Suitable ligands, antibodies, methods and reagents for use in an antigen-based assay is as described in AU 2008902611.

The combined NAA-antigen based assay format according to any embodiment herein is useful for detecting a past or present (i.e., active) infection or a latent infection by an organism of the M. tuberculosis complex e.g., M. tuberculosis in a subject, wherein said infection is determined by the presence of a nucleic acid encoding KARI protein using the ilvC-NAA based assay and is confirmed by the binding of the ligand in an antigen-based assay to KARI protein or an immunogenic fragment or epitope thereof present in a biological sample obtained from the subject.

The combined NAA-antigen based assay format according to any embodiment herein is useful for identifying a bacterium of the M. tuberculosis complex or cells infected by a bacterium of the M. tuberculosis complex or for sorting or counting of said bacterium or said cells. This example clearly encompasses the identification of a plurality of bacteria of the M. tuberculosis complex e.g., M. tuberculosis and/or M. bovis and/or M. africanum and/or M. canetti and/or M. microti.

The combined NAA-antigen based assay format as described according to any embodiment is useful for subjects who are not immunocompromised, e.g., HIV-negative subjects, the assay is also particularly useful for detecting TB in a subject that is immune compromised or immune deficient, e.g., a subject that is infected with human immune-deficiency virus (i.e., “HIV+”). The samples used for conducting such assays include, for example, (i) an extract from a tissue selected from the group consisting of brain, breast, ovary, lung, colon, pancreas, testes, liver, muscle, bone and mixtures thereof; (ii) body fluid(s) selected from the group consisting of sputum, serum, plasma, whole blood, saliva, urine, pleural fluid and mixtures thereof; and (iii) samples derived from body fluid(s) selected from the group consisting of sputum, serum, plasma, whole blood, saliva, urine, pleural fluid and mixtures thereof.

Preferred samples may comprise circulating immune complexes comprising the KARI protein or fragments thereof complexed with human immune-globulin. The detection of such immune complexes is clearly within the scope of the present invention. In accordance with this embodiment, a capture reagent e.g., a capture antibody is used to capture the KARI antigen (KARI protein, polypeptide or an immune-active fragment or epitope thereof) complexed with the subject's immune-globulin, in addition to isolated antigen in the subject's circulation. Anti-Ig antibodies, optionally conjugated to a detectable label, are used to specifically bind the captured CIC thereby detecting CIC patient samples. Within the scope of this invention, the anti-Ig antibody binds preferentially to IgM, IgA or IgG in the sample. In a particularly preferred embodiment, the anti-Ig antibody binds to human Ig, e.g., human IgA, human IgG or human IgM. The anti-Ig antibody may be conjugated to any standard detectable label known in the art. This is particularly useful for detecting infection by a pathogenic agent, e.g., a bacterium or virus, or for the diagnosis of any disease or disorder associated with CICs. Accordingly, the diagnostic methods described according to any embodiment herein are amenable to a modification wherein the sample derived from the subject comprises one or more circulating immune complexes comprising immune-globulin (Ig) bound to KARI protein of Mycobacterium tuberculosis or one or more immunogenic KARI peptides, fragments or epitopes thereof and wherein detecting the formation of an antigen-antibody complex comprises contacting an anti-Ig antibody with an immune-globulin moiety of the circulating immune complex(es) for a time and under conditions sufficient for a complex to form than then detecting the bound anti-Ig antibody.

### NAA-Antibody-Based Assays

It is also contemplated that the diagnosis of tuberculosis or an infection by M. tuberculosis in a subject obtained using the method of the invention further comprises a confirmation of diagnosis by detecting in a biological sample from said subject antibodies that bind to a KARI protein or an immunogenic fragment or epitope thereof, wherein the presence of said antibodies in the sample is indicative of infection. The infection may be a past or present infection, or a latent infection.

In a further example, the ilvC-NAA diagnostic and prognostic assays as described according to any embodiment herein are combined with an antibody-based assay for the diagnosis of tuberculosis or an infection by one or more Mycobacteria of the M. tuberculosis complex in a subject, wherein said antibody-based assay comprises detecting in a biological sample from said subject antibodies that bind to an immunogenic KARI protein or an immunogenic KARI peptide or immunogenic KARI fragment or epitope thereof, the presence of said antibodies in the sample being indicative of infection. Combined assays are performed simultaneously or in any order e.g., NAA followed by antibody-based detection or antibody-based detection followed by NAA. The infection may be a past or active infection, or a latent infection; however in a preferred embodiment, this assay format is particularly useful for detecting active infection and/or recent infection.

For example, the antibody-based assay may be an immunoassay, e.g., comprising contacting a biological sample derived from the subject with the isolated or recombinant immunogenic KARI protein or an immunogenic KARI peptide or immunogenic KARI fragment or epitope thereof according to any embodiment described herein or a combination or mixture of said peptides or epitopes or fragments for a time and under conditions sufficient for an antigen-antibody complex to form and then detecting the formation of an antigen-antibody complex. The sample is an antibody-containing sample e.g., a sample that comprises blood or serum or plasma or an immune-globulin fraction obtained from the subject. The sample may contain circulating antibodies in the form of complexes with KARI antigenic fragments. Generally, the antigen-antibody complex will be detected in such assay formats using antibodies capable of binding to the patient's immunoglobulin e.g., anti-human Ig antibodies. Suitable antibodies, methods and reagents for use in an antibody-based assay is as described in AU 2008902611.

Preferably, a subject undergoing NAA-antibody-based testing is suspected of suffering from tuberculosis or an infection by M. tuberculosis and/or is at risk of developing tuberculosis and/or at risk of being infected by M. tuberculosis.

Antibody-based assays are primarily used for confirming active infections by M. tuberculosis. Preferably, the clinical history of the subject is considered due to residual antibody levels that may persist in recent past infections or chronic infections by M. tuberculosis.

The format is inexpensive and highly sensitive, however not as useful as an antigen-based assay format for confirming a detection of infection in immune-compromised individuals. However, antibody-based assays are clearly useful for confirming a detection of M. tuberculosis infections in HIV⁻ or HIV⁺ individuals who are not immune-compromised.

The antibody-based assay is as described in AU 2008902611 and comprises contacting a biological sample derived from the subject with a KARI protein or an immunogenic fragment or epitope thereof and detecting the formation of an antigen-antibody complex.

In the antibody based assays, it is preferred that the KARI protein or immunogenic fragment or epitope thereof used to detect the antibodies is not highly cross-reactive with anti-sera from non-infected subjects. Accordingly, isolated or recombinant KARI is preferred for use in the antibody-based platforms described herein.

In another embodiment, the method of the invention comprising the antibody-based assays are useful for confirming the determination of progression of tuberculosis or an infection by M. tuberculosis in a subject, as determined by the NAA-based assay described according to any embodiment herein. In accordance with these prognostic applications of the invention, the amount of antibodies that bind to a KARI protein or fragment or epitope in blood or serum, plasma, or an immune-globulin fraction from the subject is positively correlated with the infectious state. For example, a level of the anti-KARI protein antibodies thereto that is less than the level of the anti-KARI protein antibodies detectable in a subject suffering from the symptoms of tuberculosis or an infection indicates that the subject is recovering from the infection. Similarly, a higher level of the antibodies in a sample from the subject compared to a healthy individual indicates that the subject has not been rendered free of the disease or infection.

In a further embodiment, the method of the invention comprising the antibody-based assays confirm the determination of the response of a subject having tuberculosis or an infection by M. tuberculosis to treatment with a therapeutic compound for said tuberculosis or infection, said method further comprising detecting antibodies that bind to a KARI protein or an immunogenic fragment or epitope thereof in a biological sample from said subject, wherein a level of the antibodies that is enhanced compared to the level of the antibodies detectable in a normal or healthy subject indicates that the subject is not responding to said treatment or has not been rendered free of disease or infection. Again, isolated or recombinant KARI protein is preferred.

In an alternative embodiment, the method of the invention comprising the antibody-based assays confirm the determination of the response of a subject having tuberculosis or an infection by M. tuberculosis to treatment with a therapeutic compound for said tuberculosis or infection, said method further comprising detecting antibodies that bind to a KARI protein or an immunogenic fragment or epitope thereof in a biological sample from said subject, wherein a level of the antibodies that is lower than the level of the antibodies detectable in a subject suffering from tuberculosis or infection by M. tuberculosis indicates that the subject is responding to said treatment or has been rendered free of disease or infection.

The amount of an antibody against the KARI protein or fragment that is detected in a biological sample from a subject with tuberculosis may be compared to a reference sample, wherein the reference sample is derived from one or more healthy subjects who have not been previously infected with M. tuberculosis or not recently-infected with M. tuberculosis. Such negative control subjects will have a low circulating antibody titer making them suitable standards in antibody-based assay formats. For example, antibodies that bind to a KARI protein or immunogenic fragment thereof are not detected in the reference sample and only in a patient sample, indicating that the patient from whom the sample was derived is suffering from tuberculosis or infection by M. tuberculosis or will develop an acute infection. Isolated or recombinant KARI protein is preferred for use in such embodiments.

In one embodiment of the diagnostic/prognostic methods described utilizing antibody-based assays, the biological sample is obtained previously from the subject. In accordance with such an embodiment, the prognostic or diagnostic method is performed ex vivo.

In yet another embodiment, the subject diagnostic/prognostic methods described utilizing antibody-based assays further comprise processing the sample from the subject to produce a derivative or extract that comprises the analyte (e.g., blood, serum, plasma, or any immune-globulin-containing sample).

Suitable samples for use in the antibody-based assays include, for example, extracts from tissues comprising an immune-globulin such as blood, bone, or body fluids such as serum, plasma, whole blood, an immune-globulin-containing fraction of serum, an immune-globulin-containing fraction of plasma, an immune-globulin-containing fraction of blood.

### Detection Systems for Antigen and Antibody Based Assays

Preferred detection systems contemplated herein include any known assay for detecting proteins or antibodies in a biological sample isolated from a human subject, such as, for example, SDS/PAGE, isoelectric focusing, 2-dimensional gel electrophoresis comprising SDS/PAGE and isoelectric focusing, an immune-assay, a detection based system using an antibody or non-antibody ligand of the protein, such as, for example, a small molecule (e.g. a chemical compound, agonist, antagonist, allosteric modulator, competitive inhibitor, or non-competitive inhibitor, of the protein). In accordance with these embodiments, the antibody or small molecule may be used in any standard solid phase or solution phase assay format amenable to the detection of proteins. Optical or fluorescent detection, such as, for example, using mass spectrometry, MALDI-TOF, biosensor technology, evanescent fiber optics, or fluorescence resonance energy transfer, is clearly encompassed by the present invention. Assay systems suitable for use in high throughput screening of mass samples, particularly a high throughput spectroscopy resonance method (e.g. MALDI-TOF, electrospray MS or nano-electrospray MS), are particularly contemplated.

Immunoassay formats are particularly preferred, e.g., selected from the group consisting of, an immune-blot, a Western blot, a dot blot, an enzyme linked immune-sorbent assay (ELISA), radioimmunoassay (RIA), enzyme immunoassay. Modified immunoassays utilizing fluorescence resonance energy transfer (FRET), isotope-coded affinity tags (ICAT), mass spectrometry, e.g., matrix-assisted laser desorption/ionization time of flight (MALDI-TOF), electrospray ionization (ESI), biosensor technology, evanescent fiber-optics technology or protein chip technology are also useful.

Preferably, the assay is a semi-quantitative assay or quantitative assay.

Standard solid phase ELISA formats are particularly useful in determining the concentration of a protein or antibody from a variety of patient samples.

In one form such as an assay involves immobilising a biological sample comprising anti-KARI protein antibodies, or alternatively KARI protein or an immunogenic fragment thereof, onto a solid matrix, such as, for example a polystyrene or polycarbonate microwell or dipstick, a membrane, or a glass support (e.g. a glass slide).

In the case of an antigen-based assay, an immobilised antibody that specifically binds a KARI protein is brought into direct contact with the biological sample, and forms a direct bond with any of its target protein present in said sample. For an antibody-based assay, an immobilised isolated or recombinant KARI protein or an immunogenic fragment or epitope thereof will be contacted with the biological sample. The added antibody or protein in solution is generally labelled with a detectable reporter molecule, such as for example, colloidal gold, a fluorescent label (e.g. FITC or Texas Red) or an enzyme (e.g. horseradish peroxidase (HRP)), alkaline phosphatase (AP) or β-galactosidase).

Alternatively, or in addition, a second labelled antibody can be used that binds to the first antibody or to the isolated/recombinant KARI antigen. Following washing to remove any unbound antibody or KARI antigen, the label may be detected either directly, in the case of a fluorescent label, or through the addition of a substrate, such as for example hydrogen peroxide, TMB, or toluidine, or 5-bromo-4-chloro-3-indol-beta-D-galaotopyranoside (x-gal).

Such ELISA based systems are particularly suitable for quantification of the amount of a protein or antibody in a sample, such as, for example, by calibrating the detection system against known amounts of a standard.

In another form, an ELISA consists of immobilizing an antibody that specifically binds a KARI protein on a solid matrix, such as, for example, a membrane, a polystyrene or polycarbonate microwell, a polystyrene or polycarbonate dipstick or a glass support. A patient sample is then brought into physical relation with said antibody, and the antigen in the sample is bound or ‘captured’. The bound protein can then be detected using a labelled antibody. For example if the protein is captured from a human sample, an anti-human Ig antibody is used to detect the captured protein.

An exemplary assay comprises:

(i) immobilizing an antibody that specifically binds an immunogenic KARI peptide or KARI protein to a solid matrix or support;
(ii) contacting the bound antibody with a sample obtained from a subject, preferably an antibody-containing sample such as blood, serum or Ig-containing fraction thereof for a time and under conditions sufficient for the immobilized antibody to bind to a KARI protein or fragment thereof in the sample thereby forming an antigen-antibody complex; and
(iii) detecting the antigen-antibody complex formed in a process comprising contacting said complex with an antibody that recognizes human Ig, wherein the presence of said human Ig indicates the presence of M. tuberculosis in the patient sample.

Specificity of the immobilized antibody ensures that isolated or immune-complexed KARI protein or fragments comprising the epitope that the antibody recognizes actually bind, whilst specificity of anti-human Ig ensures that only immune-complexed KARI protein or fragment is detected. In this context, the term “immunocomplexed” shall be taken to mean that the KARI protein or fragments thereof in the patient sample are complexed with human Ig such as human IgA or human IgM or human IgG, etc. Accordingly, this embodiment is particularly useful for detecting the presence of M. tuberculosis or an infection by M. tuberculosis that has produced an immune response in a subject. By appropriately selecting the detection antibody, e.g., anti-human IgA or anti-human IgG or anti-human IgM, it is further possible to isotype the immune response of the subject. Such detection antibodies that bind to human IgA, IgM and IgG are publicly available to the art.

Alternatively or in addition to the preceding embodiments, a third labelled antibody can be used that binds the second (detecting) antibody.

It will be apparent to the skilled person that the assay formats described herein are amenable to high throughput formats, such as, for example automation of screening processes, or a microarray format as described in Mendoza et al, Biotechniques 27(4): 778-788, 1999. Furthermore, variations of the above described assay will be apparent to those skilled in the art, such as, for example, a competitive ELISA.

Alternatively, the presence of anti-KARI protein antibodies, or alternatively a KARI protein or an immunogenic fragment thereof, is detected using a radioimmune-assay (RIA). The basic principle of the assay is the use of a radiolabelled antibody or antigen to detect antibody antigen interactions. For example, an antibody that specifically binds to a KARI protein can be bound to a solid support and a biological sample brought into direct contact with said antibody. To detect the bound antigen, an isolated and/or recombinant form of the antigen is radiolabelled is brought into contact with the same antibody. Following washing the amount of bound radioactivity is detected. As any antigen in the biological sample inhibits binding of the radiolabelled antigen the amount of radioactivity detected is inversely proportional to the amount of antigen in the sample. Such an assay may be quantitated by using a standard curve using increasing known concentrations of the isolated antigen.

As will be apparent to the skilled artisan, such an assay may be modified to use any reporter molecule, such as, for example, an enzyme or a fluorescent molecule, in place of a radioactive label.

Western blotting is also useful for detecting a KARI protein or an immunogenic fragment thereof. In such an assay, protein from a biological sample is separated using sodium dodecyl sulphate (SDS) polyacrylamide gel electrophoresis (SD S-PAGE) using techniques well known in the art and described in, for example, Scopes (In: Protein Purification: Principles and Practice, Third Edition, Springer Verlag, 1994). Separated proteins are then transferred to a solid support, such as, for example, a membrane or more specifically, nitrocellulose membrane, nylon membrane or PVDF membrane, using methods well known in the art, for example, electrotransfer. This membrane may then be blocked and probed with a labelled antibody or ligand that specifically binds a KARI protein. Alternatively, a labelled secondary, or even tertiary, antibody or ligand can be used to detect the binding of a specific primary antibody.

High-throughput methods for detecting the presence or absence of anti-KARI protein antibodies, or alternatively KARI protein or an immunogenic fragment thereof are particularly preferred.

In one embodiment, mass spectrometry, e.g., MALDI-TOF is used for the rapid identification of a protein that has been separated by either one- or two-dimensional gel electrophoresis. Accordingly, there is no need to detect the proteins of interest using an antibody or ligand that specifically binds to the protein of interest. Rather, proteins from a biological sample are separated using gel electrophoresis using methods known in the art and those proteins at approximately the correct molecular weight and/or isoelectric point are analysed using MALDI-TOF to determine the presence or absence of a protein of interest.

Alternatively, mass spectrometry, e.g., MALDI or ESI, or a combination of approaches is used to determine the concentration of a particular protein in a biological sample, such as, for example sputum. Such proteins are preferably well characterised previously with regard to parameters such as molecular weight and isoelectric point.

Biosensor devices generally employ an electrode surface in combination with current or impedance measuring elements to be integrated into a device in combination with the assay substrate (such as that described in U.S. Pat. No. 5,567,301). An antibody or ligand that specifically binds to a protein of interest is preferably incorporated onto the surface of a biosensor device and a biological sample isolated from a patient (for example sputum that has been solubilized using the methods described herein) contacted to said device. A change in the detected current or impedance by the biosensor device indicates protein binding to said antibody or ligand. Some forms of biosensors known in the art also rely on surface plasmon resonance to detect protein interactions, whereby a change in the surface plasmon resonance surface of reflection is indicative of a protein binding to a ligand or antibody (U.S. Pat. Nos. 5,485,277 and 5,492,840).

Biosensors are of particular use in high throughput analysis due to the ease of adapting such systems to micro- or nano-scales. Furthermore, such systems are conveniently adapted to incorporate several detection reagents, allowing for multiplexing of diagnostic reagents in a single biosensor unit. This permits the simultaneous detection of several epitopes in a small amount of body fluids.

Evanescent biosensors are also preferred as they do not require the pre-treatment of a biological sample prior to detection of a protein of interest. An evanescent biosensor generally relies upon light of a predetermined wavelength interacting with a fluorescent molecule, such as for example, a fluorescent antibody attached near the probe's surface, to emit fluorescence at a different wavelength upon binding of the diagnostic protein to the antibody or ligand.

To produce protein chips, the proteins, peptides, polypeptides, antibodies or ligands that are able to bind specific antibodies or proteins of interest are bound to a solid support such as for example glass, polycarbonate, polytetrafluoroethylene, polystyrene, silicon oxide, metal or silicon nitride. This immobilization is either direct (e.g. by covalent linkage, such as, for example, Schiff's base formation, disulfide linkage, or amide or urea bond formation) or indirect. Methods of generating a protein chip are known in the art and are described in for example U.S. Patent Application No. 20020136821, 20020192654, 20020102617 and U.S. Pat. No. 6,391,625. In order to bind a protein to a solid support it is often necessary to treat the solid support so as to create chemically reactive groups on the surface, such as, for example, with an aldehyde-containing silane reagent. Alternatively, an antibody or ligand may be captured on a microfabricated polyacrylamide gel pad and accelerated into the gel using microelectrophoresis as described in, Arenkov et al. Anal. Biochem. 278:123-131, 2000.

A protein chip is preferably generated such that several proteins, ligands or antibodies are arrayed on said chip. This format permits the simultaneous screening for the presence of several proteins in a sample.

Alternatively, a protein chip may comprise only one protein, ligand or antibody, and be used to screen one or more patient samples for the presence of one polypeptide of interest. Such a chip may also be used to simultaneously screen an array of patient samples for a polypeptide of interest.

Preferably, a sample to be analysed using a protein chip is attached to a reporter molecule, such as, for example, a fluorescent molecule, a radioactive molecule, an enzyme, or an antibody that is detectable using methods well known in the art. Accordingly, by contacting a protein chip with a labelled sample and subsequent washing to remove any unbound proteins the presence of a bound protein is detected using methods well known in the art, such as, for example using a DNA microarray reader.

Alternatively, biomolecular interaction analysis-mass spectrometry (BIA-MS) is used to rapidly detect and characterise a protein present in complex biological samples at the low- to sub-femptamole (fmol) level (Nelson et al. Electrophoresis 21: 1155-1163, 2000). One technique useful in the analysis of a protein chip is surface enhanced laser desorption/ionization-time of flight-mass spectrometry (SELDI-TOF-MS) technology to characterise a protein bound to the protein chip. Alternatively, the protein chip is analysed using ESI as described in U.S. Patent Application 20020139751.

As will be apparent to the skilled artisan, protein chips are particularly amenable to multiplexing of detection reagents. Accordingly, several antibodies or ligands each able to specifically bind a different peptide or protein may be bound to different regions of said protein chip. Analysis of a biological sample using said chip then permits the detecting of multiple proteins of interest, or multiple B cell epitopes of the KARI protein. Multiplexing of diagnostic and prognostic markers is particularly contemplated in the present invention.

In a further embodiment, the samples are analysed using ICAT or ITRAC, essentially as described in US Patent Application No. 20020076739. This system relies upon the labelling of a protein sample from one source (i.e. a healthy individual) with a reagent and the labelling of a protein sample from another source (i.e. a tuberculosis patient) with a second reagent that is chemically identical to the first reagent, but differs in mass due to isotope composition. It is preferable that the first and second reagents also comprise a biotin molecule. Equal concentrations of the two samples are then mixed, and peptides recovered by avidin affinity chromatography. Samples are then analysed using mass spectrometry. Any difference in peak heights between the heavy and light peptide ions directly correlates with a difference in protein abundance in a biological sample. The identity of such proteins may then be determined using a method well known in the art, such as, for example MALDI-TOF, or ESI.

In a particularly preferred embodiment, a biological sample comprising anti-KARI protein antibodies, or alternatively KARI protein or an immunogenic fragment thereof, is subjected to 2-dimensional gel electrophoresis. In accordance with this embodiment, it is preferable to remove certain particulate matter from the sample prior to electrophoresis, such as, for example, by centrifugation, filtering, or a combination of centrifugation and filtering. Proteins in the biological sample are then separated. For example, the proteins may be separated according to their charge using isoelectric focussing and/or according to their molecular weight. Two-dimensional separations allow various isoforms of proteins to be identified, as proteins with similar molecular weight are also separated by their charge. Using mass spectrometry, it is possible to determine whether or not a protein of interest is present in a patient sample.

Other assay format s include e.g., solid phase ELISA, flow through immunoassay formats, lateral flow formats, capillary formats, and for the purification or isolation of immunogenic proteins, peptides, fragments (e.g., using a solid matrix conjugated to antibody, protein G or protein A). Suitable assay formats for use in an antibody-based or antigen-based assay are as described in AU 2008902611.

### Other Combination Testing

The ilvC nucleic acid-based test of the present invention with or without adjunct antigen-based and/or antibody-based testing as described herein is combined with one or more standard tests for diagnosing tuberculosis or a condition associated with tuberculosis.

In one example, the standard test is a culture test for the diagnosis of tuberculosis e.g., by virtue of detecting the presence of M. tuberculosis or other organisms of the M. tuberculosis complex in clinical samples e.g., to confirm an initial diagnosis and/or to indicate the specific pathogen involved. In one example, a culture test confirms the presence of M. tuberculosis in a clinical sample as opposed to another mycobacteria pathogen. In another example, a culture test confirms the strain of M. tuberculosis or other mycobacteria pathogen present in a clinical specimen obtained from a subject. It will be apparent that any known culture test that is available for detecting the presence of M. tuberculosis or other organisms of the M. tuberculosis complex in clinical samples may be used. For example, cultivation in the MGIT-960 system according to the manufacturer's standard protocol (Becton Dickinson Diagnostic Instrument Systems) may be employed.

The ilvC-NAA diagnostic and prognostic assays as described according to any embodiment herein when combined with standard culture tests for the diagnosis of tuberculosis may be useful to provide a faster result to short term culture than a culture test alone, e.g., MGIT alone. In this example, the samples are cultured e.g. in a MGIT system, and the ilvC-NAA is performed on culture samples. Primers are labelled as described herein, to produce a detectable endpoint, e.g., a colorimetric endpoint detectable in a MGIT system. In this example, it is preferred that the culture time necessary to obtain a positive result is between about 3 days to 4 weeks; or about 1 to 2 weeks; or about 1 week; or about 5 days; or about 3 days; or about 1 day.

In another example, the standard test is a smear test.

In another example, the standard test is a test for detecting the presence of one or more known markers of drug resistance (MDR) and extensively drug resistance (XDR) in one or more organisms of the M. tuberculosis complex in clinical samples obtained from a subject. It will be apparent that any known test that is available for detecting the presence of one or more MDR or XDR of M. tuberculosis or other organisms of the M. tuberculosis complex in clinical samples may be used. For example, known line-probe assays for the detection of mutations associated with drug resistance to rifampin (RIF), isoniazid (INH), and streptomycin (STR) may be used. Examples of commercially available line-probe assays that may be used include, but are not limited to, INNO-LiPA Rif. TB kit (Innogenetics NV, Gent, Belgium) and GenoType M.TBDRplus (Hain Lifescienc GmbH, Nehren, Germany).

### Biological Samples and Reference Samples

### 1. Test Samples

Preferably, the biological sample according to any embodiment as described herein is a sample selected from the group consisting of lung, lymphoid tissue associated with the lung, paranasal sinuses, bronchi, a bronchiole, alveolus, ciliated mucosal epithelia of the respiratory tract, mucosal epithelia of the respiratory tract, broncheoalveolar lavage fluid (BAL), alveolar lining fluid, sputum, mucus, saliva, blood, serum, plasma, urine, peritoneal fluid, pericardial fluid, pleural fluid, squamous epithelial cells of the respiratory tract, a mast cell, a goblet cell, a pneumocyte (type 1 or type 2), an intra epithelial dendritic cell, a PBMC, a neutrophil, a monocyte, or any immune-globulin-containing fraction of any one or more of said tissues, fluids or cells.

In one embodiment a biological sample is obtained previously from a subject.

Preferably, the subject from which the sample is obtained is suspected of suffering from tuberculosis or being infected by M. tuberculosis and/or is at risk of developing tuberculosis and/or at risk of being infected by M. tuberculosis.

In one embodiment a biological sample is obtained from a subject by a method selected from the group consisting of surgery or other excision method, aspiration of a body fluid such as hypertonic saline or propylene glycol, broncheoalveolar lavage, bronchoscopy, saliva collection with a glass tube, salivette (Sarstedt A G, Sevelen, Switzerland), Ora-sure (Epitope Technologies Pty Ltd, Melbourne, Victoria, Australia), omni-sal (Saliva Diagnostic Systems, Brooklyn, N.Y., USA) and blood collection using any method well known in the art, such as, for example using a syringe.

It is particularly preferred that a biological sample is sputum, isolated from lung of a patient using, for example the method described in Gershman, N. H. et al, J Allergy Clin Immuno., 10(4): 322-328, 1999. Preferably, the sputum is expectorated i.e., coughed naturally.

In another preferred embodiment a biological sample is plasma that has been isolated from blood collected from a patient using a method well known in the art.

### 2. Reference Samples

As will be apparent, the diagnostic and prognostic methods provided by the present invention may be further facilitated by providing a degree of quantification to determine either, the amount of DNA, RNA or a protein that is diagnostic or prognostic of an infection or disease. Such quantification can be determined by the inclusion of appropriate reference samples in the assays described herein, wherein said reference samples are derived from healthy or normal individuals.

In one embodiment, the reference sample comprises for example cells, fluids or tissues from a healthy subject who has not been previously or recently infected and is not suffering from an infection or disease. Conveniently, such reference samples are from fluids or tissues that do not require surgical resection or intervention to obtain them. Accordingly, bodily fluids and derivatives thereof are preferred. Highly preferred reference samples comprise sputum, mucus, saliva, blood, serum, plasma, urine, BAL fluid, peritoneal fluid, pericardial fluid, pleural fluid, a PBMC, a neutrophil, a monocyte, or any immunoglobulin-containing fraction of any one or more of said tissues, fluids or cells.

A reference sample and a test (or patient) sample are processed, analysed or assayed and data obtained for a reference sample and a test sample are compared. In one embodiment, a reference sample and a test sample are processed, analysed or assayed at the same time. In another embodiment, a reference sample and a test sample are processed, analysed or assayed at a different time.

In an alternate embodiment, a reference sample is not included in an assay. Instead, a reference sample may be derived from an established data set that has been previously generated. Accordingly, in one embodiment, a reference sample comprises data from a sample population study of healthy individuals, such as, for example, statistically significant data for the healthy range of the integer being tested. Data derived from processing, analysing or assaying a test sample is then compared to data obtained for the sample population.

Data obtained from a sufficiently large number of reference samples so as to be representative of a population allows the generation of a data set for determining the average level of a particular parameter. Accordingly, the amount of a nucleic acid alone, or in combination with the amount of a protein that are diagnostic or prognostic of an infection or disease can be determined for any population of individuals, and for any sample derived from said individual, for subsequent comparison to levels of the expression product determined for a sample being assayed. Where such normalized data sets are relied upon, internal controls are preferably included in each assay conducted to control for variation.

### Sample Preparation for NAA-Based Assays

It will be apparent that any method known in the art, or as commercially available protocols for extracting nucleic acids from a biological sample is suitable for use in the method of the invention. For example, total RNA may be extracted according to manufacturers protocols using commercially available reagents e.g., Trizol™ (Invitrogen) or using commercially available kits for DNA and RNA extraction, e.g., Perfect RNA™ Eukaryotic Kit (Eppendorf A G, Hamburg, D E); MasterPure™ Complete DNA and RNA purification kits (Epicentre Biotechnologies, Madison, Wis.).

### Sample Preparation for Antigen and or Antibody-Based Assays of Combined Assay Formats

In one embodiment, a biological sample is treated to lyse a cell in said sample. Such methods include the use of detergents, enzymes, repeatedly freezing and thawing said cells, sonication or vortexing said cells in the presence of glass beads, amongst others.

In another embodiment, a biological sample is treated to denature a protein present in said sample. Methods of denaturing a protein include heating a sample, treating a sample with 2-mercaptoethanol, dithiotreitol (DTT), N-acetylcysteine, detergent or other compound such as, for example, guanidinium or urea. For example, the use of DTT is preferred for liquefying sputum.

In yet another embodiment, a biological sample is treated to concentrate a protein is said sample. Methods of concentrating proteins include precipitation, freeze drying, use of funnel tube gels (TerBush and Novick, Journal of Biomolecular Techniques, 10(3); 1999), ultrafiltration or dialysis.

Diagnostic/Prognostic Methods for Detecting Tuberculosis or M. tuberculosis Infection

This invention provides a method of diagnosing tuberculosis or an infection by M. tuberculosis in a subject comprising detecting in a biological sample from said subject one or more ilvC nucleic acids of one or more Mycobacteria of the M. tuberculosis complex present in a biological sample, wherein the presence of said ilvC nucleic acid in the sample is indicative of infection.

One advantage of detecting ilvC nucleic acids, e.g. based on expression of the ilvC gene, as opposed to other NAA based tests is that the reflects antigen levels, that may not be detectable by other means, and should reflect bacilli burden as the product of the ilvC gene, KARI protein being a product of the bacilli, are a direct method of detecting its presence. Another advantage compared to e.g., an antibody-based assay is that severely immune-compromised patients may not produce antibody at detectable levels, and the level of the antibody in any patient does not reflect bacilli burden. A further advantage over other NAA-based tests using for example, DNA detection, is that the method of the invention provides the means to detect mRNA transcript levels, which also reflect the presence of active infection.

In another embodiment, the NAA-based diagnostic assays of the invention are useful for determining the progression of tuberculosis or an infection by M. tuberculosis in a subject. In accordance with these prognostic applications of the invention, the level of expression of the ilvC gene, e.g., a ilvC transcript in a biological sample is positively correlated with the infectious state. For example, a level of the ilvC transcript that is less than the level of the ilvC transcript detectable in a subject suffering from the symptoms of tuberculosis or an infection indicates that the subject is recovering from the infection. Similarly, a higher level of the ilvC transcript in a sample from the subject compared to a healthy individual indicates that the subject has not been rendered free of the disease or infection.

Accordingly, a further embodiment of the present invention provides a method for determining the response of a subject having tuberculosis or an infection by M. tuberculosis to treatment with a therapeutic compound for said tuberculosis or infection, said method comprising detecting ilvC transcript in a biological sample from said subject, wherein a level of ilvC transcript that is enhanced compared to the level of that protein or fragment or epitope detectable in a normal or healthy subject indicates that the subject is not responding to said treatment or has not been rendered free of disease or infection.

In an alternative embodiment, the present invention provides a method for determining the response of a subject having tuberculosis or an infection by M. tuberculosis to treatment with a therapeutic compound for said tuberculosis or infection, said method comprising detecting a ilvC transcript in a biological sample from said subject, wherein a level of the ilvC transcript is lower than the level of the protein or fragment or epitope detectable in a subject suffering from tuberculosis or infection by M. tuberculosis indicates that the subject is responding to said treatment or has been rendered free of disease or infection. Clearly, if the level ilvC transcript is not detectable in the subject, the subject has responded to treatment.

In a further embodiment, the amount of an ilvC transcript in a biological sample derived from a patient is compared to the amount of the same ilvC transcript detected in a biological sample previously derived from the same patient. As will be apparent to a person skilled in the art, this method may be used to continually monitor a patient with a latent infection or a patient that has developed tuberculosis. In this way a patient may be monitored for the onset or progression of an infection or disease, with the goal of commencing treatment before an infection is established, particularly in an HIV+ individual.

Alternatively, or in addition, the amount of an ilvC transcript detected in a biological sample derived from a subject with tuberculosis may be compared to a reference sample, wherein the reference sample is derived from one or more tuberculosis patients that do not suffer from an infection or disease or alternatively, one or more tuberculosis patients that have recently received successful treatment for infection and/or one or more subjects that do not have tuberculosis and that do not suffer from an infection or disease.

In one embodiment, ilvC transcript is not detected in a reference sample, however, said ilvC transcript is detected in the patient sample, indicating that the patient from whom the sample was derived is suffering from tuberculosis or infection by M. tuberculosis or will develop an acute infection.

Alternatively, the amount of ilvC transcript may be enhanced in the patient sample compared to the level detected in a reference sample. Again, this indicates that the patient from whom the biological sample was isolated is suffering from tuberculosis or infection by M. tuberculosis or will develop an acute infection.

In one embodiment of the diagnostic/prognostic methods described herein, the biological sample is obtained previously from the subject. In accordance with such an embodiment, the prognostic or diagnostic method is performed ex vivo.

In yet another embodiment, the subject diagnostic/prognostic methods further comprise processing the sample from the subject to produce a derivative or extract that comprises the analyte (e.g., pleural fluid or sputum or serum).

Suitable samples include extracts from tissues such as brain, breast, ovary, lung, colon, pancreas, testes, liver, muscle and bone tissues, or body fluids such as sputum, serum, plasma, whole blood, sera or pleural fluid.

Preferably, the biological sample is a bodily fluid or tissue sample selected from the group consisting of saliva, plasma, blood, serum, sputum, urine, and lung. Other samples are not excluded.

In fact, the ilvC-NAA based assay according to any embodiment herein is useful for detecting a past or present (i.e., active) infection or a latent infection by an organism of the M. tuberculosis complex e.g., M. tuberculosis in biological sample from a subject, wherein said infection is determined by the presence of a nucleic acid encoding KARI protein of any one or more of the bacterium of the M. tuberculosis complex. A past infection may be determined by detection in of the nucleic acid in samples such as blood or urine for example. Acute infection is detectable in samples such as sputum, or bronchial lavage samples for example. A past infection may be determined by detection in of the nucleic acid in samples such as blood or urine for example, and compared to sputum samples, wherein negative detection in sputum samples and positive detection in serum may indicate a past infection. This example clearly encompasses the identification of a plurality of nucleic acids encoding KARI protein of bacteria of the M. tuberculosis complex e.g., M. tuberculosis and/or M. bovis and/or M. africanum and/or M. canetti and/or M. microti.

One or more ilvC-NAA based assays also provide a means for diagnosing pulmonary tuberculosis e.g., by the use of sputum, and bronchial lavage samples. One or more ilvC-NAA based tests also provide a means for diagnosing extrapulmonary tuberculosis e.g., by the use of blood, serum, a fraction of serum, etc., or urine samples.

In a preferred embodiment, the subject is suspected of suffering from tuberculosis or an infection by one or more Mycobacteria of the M. tuberculosis complex and/or the subject is at risk of developing tuberculosis or at risk of being infected by said one or more Mycobacteria e.g., M. tuberculosis and/or M. bovis and/or M. africanum and/or M. canetti and/or M. microti.

A subject suspected of suffering from tuberculosis or an infection by one or more Mycobacteria of the M. tuberculosis complex displays one or more symptoms of tuberculosis or such infection, such as, for example, fever, productive cough, haemoptysis (blood in the sputum), chest pain, night sweats, weight loss, malaise, cavitations and/or calcification of the nodes of the lungs. A subject suspected of suffering from tuberculosis or such infection may have been exposed to one or a plurality of bacteria of the M. tuberculosis complex e.g., M. tuberculosis and/or M. bovis and/or M. africanum and/or M. canetti and/or M. microti. e.g. by virtue of having come into contact with a person suffering from tuberculosis.

A subject at risk of developing tuberculosis is a subject that is exposed to a condition or suffers from a condition that increases the risk of developing tuberculosis or being infected by one or more bacteria of the M. tuberculosis complex such subjects include a subject who has come into contact with a person suffering from tuberculosis, a subject that has traveled in a country or region in which tuberculosis is common and/or the causative agent(s) prevalent (e.g. South Africa), a subject that works in a hospital or nursing facility, a subject infected with HIV-1 or HIV-2, a subject that uses corticosteroids, an immuno-compromised or immuno-suppressed subject, a subject that suffers from silicosis or a subject suffering from a latent infection by one or more Mycobacteria of the M. tuberculosis complex, e.g., M. tuberculosis and/or M. bovis and/or M. africanum and/or M. canetti and/or M. microti.

As used herein, the term “infection” shall be understood to mean invasion and/or colonisation by a microorganism and/or multiplication of a micro-organism, in particular, a bacterium or a virus, in the respiratory tract of a subject. Such an infection may be unapparent or result in local cellular injury. The infection may be localised, subclinical and temporary or alternatively may spread by extension to become an acute or chronic clinical infection. The infection may also be a past infection wherein residual nucleic acid(s) derived and/or encoded from an ilvC remain in the host. The infection may also be a latent infection, in which the microorganism is present in a subject, however the subject does not exhibit symptoms of disease associated with the organism. Preferably, the infection is a pulmonary or extra-pulmonary infection by M. tuberculosis, and more preferably an extra-pulmonary infection. By “pulmonary” infection is meant an infection of the airway of the lung, such as, for example, an infection of the lung tissue, bronchi, bronchioles, respiratory bronchioles, alveolar ducts, alveolar sacs, or alveoli. By “extra-pulmonary” is meant outside the lung, encompassing, for example, kidneys, lymph, urinary tract, bone, skin, spinal fluid, intestine, peritoneal, pleural and pericardial cavities.

The ilvC-NAA based assay according to any embodiment herein is useful for subjects who are not immunocompromised, e.g., HIV-negative subjects, the assay is also particularly useful for detecting TB in a subject that is immune compromised or immune deficient, e.g., a subject that is infected with human immuno-deficiency virus (i.e., “HIV+”). The samples used for conducting such assays include, for example, (i) an extract from a tissue selected from the group consisting of brain, breast, ovary, lung, colon, pancreas, testes, liver, muscle, bone and mixtures thereof; (ii) body fluid(s) selected from the group consisting of sputum, serum, plasma, whole blood, saliva, urine, pleural fluid and mixtures thereof; and (iii) samples derived from body fluid(s) selected from the group consisting of sputum, serum, plasma, whole blood, saliva, urine, pleural fluid and mixtures thereof.

The ilvC-NAA based assay according to any embodiment herein is useful for identifying a bacterium of the M. tuberculosis complex or cells infected by a bacterium of the M. tuberculosis complex or for sorting or counting of said bacterium or said cells. This example clearly encompasses the identification of a plurality of bacteria of the M. tuberculosis complex e.g., M. tuberculosis and/or M. bovis and/or M. africanum and/or M. canetti and/or M. microti.

### EXEMPLIFICATION

The present invention will now be illustrated by the following Examples and/or Drawings, which are not intended to be limiting in any way. The teaching of all references cited herein are incorporated herein by reference.

### Example 1

### Sample Collection, Processing, Nucleic Acid Extraction, Synthesis, and Quantatitation, Antibody Production and Immunoassays

Subject to the disclosure in the subsequent examples i.e., Example 2 et seq., the following general methods were employed for sample collection and nucleic acid extraction, processing, synthesis, and quantatitation. A method referred to in this example has been utilized unless an alternate method is specifically recited in a subsequent example i.e., Example 2 et seq. Methods referred to in the subsequent examples are to be construed with reference to that specific example.

### 1. Collection of Patient Sputum Samples

TB-negative and TB-positive sputa were used to evaluate nucleic acid based assays using primer pairs, with optional antigen based assay and with optional antibody based assay for a TB diagnostic as described in the subsequent examples. Eighty (80) patient sputa samples were recruited from Cameroon in 2007. Samples were treated with protease inhibitors and frozen at −30° C.

Similarly unprocessed sputa were also obtained from Becton, Dickinson & CO., Research Triangle Park, Durham, N.C., USA and are referred to herein as “Sputum-BD”. Additional samples were collected from alternate sites in Johannesburg, South Africa, Australia, and from Health Concepts International Limited, Thailand.

### 2. Pre-Treatment of Sputa

### a) “Sputum-M1”

In one example, sputa fractions referred to herein as “sputum-M1” were prepared by diluting collected sputum 1:1 (v/v) to a final concentration of 10 mM freshly-made Dithiothreitol (DTT) in 50 mM phosphate buffer pH 7.4. EDTA-free protease inhibitor cocktail tablets (Roche Molecular Biochemicals, Cat#1873580) were added according to the manufacturer's or supplier's instructions as appropriate to provide a final 1:4 (v/v) dilution of sputum. Samples were agitated by vortexing for about 30 seconds, and then mixed for about 30 min at 4° C. using an orbital shaker or gentle vortexing, taking care to avoid substantial cell lysis. The liquefied sputum was then centrifuged at 2,000×g for about 10 min at 4° C. to pellet cells and remove insoluble material. The supernatant is centrifuged at 14,000×g for 10 min at 4° C., to pellet fine particulate matter. The supernatant is removed and filtered using a 0.2 μm pore size GD/X PVDF sterile filter and the filtrate retained and stored frozen at −20° C.

### b) “Sputum-C1”

In a further example, sputa fractions referred to herein as “sputum-C1” are prepared by diluting collected sputum 1:1 (v/v) to a final concentration of 10 mM freshly-made Dithiothreitol (DTT) in 50 mM phosphate buffer pH 7.4. EDTA-free protease inhibitor cocktail tablets (Roche Molecular Biochemicals, Cat#1873580) are added according to the manufacturer's or supplier's instructions as appropriate to provide a final 1:2 (v/v) dilution of sputum. Samples are agitated by vortexing for about 30 seconds, and then mixed for about 30 min at 4° C. using an orbital shaker or gentle vortexing, taking care to avoid substantial cell lysis. The liquefied sputum is then centrifuged at 2,000×g for about 10 min at 4° C. to pellet cells and remove insoluble material. The supernatant is centrifuged at 14,000×g for 10 min at 4° C., to pellet fine particulate matter. The supernatant is removed and stored frozen at −20° C. without filtration.

### 3. Processing of Frozen Sputa for Immuno Assay

Four alternative processes were employed for further processing of frozen pre-treated sputa prepared as described herein above.

### a) Method 1

Sputum-M1 (2.5 mL) prepared as described herein above is equivalent to about 0.6 mL of undiluted (“neat”) sputum. In this exemplary method, sputum-M1 is unprocessed further for assay in a replacement ELISA using 17×150 uL replacements.

### b) Method 2

Sputum-C1 (1.8 mL) prepared as described above is equivalent to 0.9 mL of undiluted (“neat”) sputum. In this exemplary method, sputum-C1 is reduced to 0.6 mL volume by acetone precipitation for assay in a replacement ELISA using 4×150 uL replacements. In particular, Sputum-C1 is centrifuged to remove insoluble material, the supernatant is transferred into a fresh tube and four (4) volumes of cold acetone are added, and samples incubated at −80° C. for 30 min, after which time they are centrifuged at 4,000×g at 4° C. for 30 min to collect the precipitated protein fraction. The protein pellet is retained and air-dried for about 30 min, re-dissolved gently in 0.6 mL of 50 mM Tris pH 7.8, 5 mM MgCl₂.

### c) Method 3

Sputum-C1 (9 mL) prepared as described above is equivalent to 4.5 mL of undiluted (“neat”) sputum. In this exemplary method, sputum-C1 is size fractionated, desalted and made to about 0.6 mL volume, for assay in a replacement ELISA using 4×150 uL replacements. Briefly, frozen Sputum-C1 is thawed, adjusted to a final concentration of 0.3 mM EDTA, and 4 mL of 50 mM Tris pH 7.8, 5 mM MgCl₂ added. The samples are centrifuged at 4,000×g at ambient temperature for 20 min to pellet insoluble material. The supernatant is retained, transferred to a fresh tube, diluted in an equal volume of 50 mM Tris pH 7.8, 5 mM MgCl₂, applied to a 100 kDa MW cut-off size exclusion spin column, and centrifuged at 4,000×g at ambient temperature for 25 min. The eluate is retained and transferred to a 5 kDa MW cut-off size-exclusion spin column, and centrifuged at 4,000×g (ambient temperature) for at least about 60 min or until ˜0.6 mL eluate is collected. Sample volumes are adjusted to about 0.62 mL using with 50 mM Tris pH 7.8, 5 mM MgCl₂ for assay in replacement ELISA as described above.

### d) Method 4

Sputum-M1 (18 mL) prepared as described above is equivalent to 4.5 mL of undiluted (“neat”) sputum. In this exemplary method, sputum- is size fractionated, desalted and made to about 0.6 mL volume, for assay in a replacement ELISA using 4×150 uL replacements. Briefly, frozen Sputum-M1 is thawed, adjusted to a final concentration of 0.3 mM EDTA, and 4 mL of 50 mM Tris pH 7.8, 5 mM MgCl₂ added. The samples are centrifuged at 4,000×g at ambient temperature for 20 min to pellet insoluble material. The supernatant is retained, transferred to a fresh tube, diluted in an equal volume of 50 mM Tris pH 7.8, 5 mM MgCl₂, applied to a 100 kDa MW cut-off size exclusion spin column, and centrifuged at 4,000×g at ambient temperature for 25 min. The eluate is retained and transferred to a 5 kDa MW cut-off size-exclusion spin column, and centrifuged at 4,000×g (ambient temperature) for at least about 60 min or until ˜0.6 mL eluate is collected. Sample volumes are adjusted to about 0.62 mL using with 50 mM Tris pH 7.8, 5 mM MgCl₂ for assay in replacement ELISA as described above.

4. DNA Extraction from Cultured M. tuberculosis

M. tuberculosis cells were incubated in breaking buffer (50 mM Tris-HCl pH 8, 10 mM EDTA, 100 mM NaCl, 0.6% SDS and proteinase K) for 12 h at 50° C. and disrupted by beadbeating. The cell lysate was centrifuged to remove debris and the DNA was extracted from the supernatant using phenol-chloroform and precipitated in isopropanol overnight. The pellet was washed with 75% ethanol and resuspended in water. The quality and quantity of the DNA was determined using a Nanodrop ND-1000 spectrophotometer (Nanodrop Technologies). This DNA was later used for primer testing and PCR to construct standard curves.

5. RNA Extraction from Sputum

Two hundred or five hundred microliter (500 μl) of sputum samples were resuspended in 50 mg/ml N-acetyl-L-cysteine (NALC) in 1.3% sodium citrate. Two volumes of Trizol (Invitrogen) were added and the samples were homogenized by bead-beating for 30 s. The aqueous phase containing RNA was extracted with 200 μl a chloroform and centrifuged at 13 000 rpm for 20 min. RNA was precipitated overnight in ice-cold isopropanol. After washing the pellet twice with 75% EtOH, the RNA was resuspended in DEPC-treated water. The sample was treated with Turbo DNAse (Ambion) and extracted again with Trizol. A 16S PCR was performed to confirm a DNA-free preparation and total RNA concentration was measured using a Nanodrop ND-1000 spectrophotometer (Nanodrop Technologies). For RNA extraction from a culture suspension, 1 ml of Trizol was added to 250 μl of frozen cells (1.9×10⁹ cells/ml) and the protocol herein was followed.

6. cDNA Synthesis

Total cDNA was prepared using a random cDNA synthesis kit (Marligen). Up to 500 ng RNA from sputum was reverse-transcribed, reaction conditions were 22° C. for 5 min, 42° C. for 2 h and 85° C. for 5 min. Up to 2.5 μl cDNA were used in subsequent real-time analysis.

### 7. Quantitative Real-Time PCR

Specific primers sets for the targets of interest were designed as described in Example 2 herein. The 16S rRNA gene was chosen as a housekeeper. Transcript levels were quantified by qRT-PCR using the Rotor-Gene 3000 system (Corbett). Triplicate reactions were set up, each containing 2 μl cDNA, 12.5 μl Platinum SYBR Green qPCR supermix UDG kit (Invitrogen), 0.5 μl ROX reference dye, 10 μmol of each primer and DEPC-treated water to a final volume of 25 μl. For samples in which no signal was obtained, the reaction was repeated using 100 ng cDNA, to ensure there was no inhibition due to an excessive amount of template. Two-step cycling was performed with initial hold of 60° C. for 5 min and 95° C. for 5 min, followed by 40 cycles at 95° C. for 15 s and 60° C. for 30 s. For Bsx detection, an annealing and extension temperature of 63° C. was used.

### 8. Standard Curve Preparation for Quantitative Real-Time PCR

Absolute quantification was performed using the method described in Whelan, J. A., N. B. Russel and M. A. Whelan. A method for the absolute quantification of cDNA using real-time PCR. Journal of Immunological Methods (2003) 278: 261-269; the disclosure of which is incorporated herein in its entirety. Briefly, M. tuberculosis genomic DNA was used as a PCR template for each primer set. The products were ethanol-precipitated and resuspended in DEPC-treated water. Serial dilutions were used in a real-time PCR reaction as described above and the results were used for the construction of standard curves. Regression lines were fitted and the equations produced were used to calculate the transcript levels in copy/μg cDNA.

### 9. Smear Status Grading

To estimate the severity of acid fast bacilli infections, sputum specimen were also subjected to Ziehl Neelsen staining and smear grading according to conventional method accepted in the art, as per the guidelines under the Revised National Tuberculosis Control Programme (Central TB Division, New Delhi: Manual for Laboratory Technicians, DGHS, Ministry of Health & Family Welfare May 1999) and as described in Selvakumar N., et al., Indian J Med Res 124: pp 439-442 (October 2006) both incorporated herein by reference in their entirety. Serial dilutions (up to 10⁶-fold dilutions) of sputa sample were prepared in up to five replicate sets. The bacterial load in each dilution (smear microscopy status) ranged from 3+ to negative, indicative of severity of infection.

10. Preparation of IgG Fractions from Sputum, Serum or Plasma

Patient sputum, serum or plasma was diluted in 8.2 ml Immune-pure IgG binding buffer (Pierce), then filtered through a 0.22 μm filter before application to a protein A column attached to an ÄKTA Explorer (Amersham Biosciences). Bound antibody was eluted with Immune-pure gentle Ag/Ab elution buffer (Pierce). The eluted fractions (IgG bound to antigens) were pooled and left on ice for 3 hours to allow dissociation of immune complexes. The IgG fraction was then separated from the antigen fraction by filtration through a 100,000 molecular weight cut off column (Millipore). Both fractions and the flow through from the protein A column were dialysed with benzoylated dialysis membrane (Sigma) against 4 litres of phosphate buffered saline pH 7.2 overnight at 4° C., then another 4 litres for 3 hours. All fractions, (flow through and retentate from the 100,000 cut off column and flow through from the protein A column) were acetone precipitated at a ratio of 10 parts acetone to 1 part sample for one hour at −20° C., then spun at 4000 g for 20 minutes. The precipitated samples were solubilized in sample buffer containing 5M urea, 2M thiourea, 2% CHAPS, 2% SB3-10 and 40 mM Tris to a final concentration of approximately 2 mg/ml, and then reduced with 5 mM tributyl phosphine and alkylated with 10 mM acrylamide for 1.5 h. The alkylation reaction was quenched with the addition of DTT to a final concentration of 10 mM. The samples were divided into 200 μl aliquots and stored at −20° C.

11. Selection of M. tuberculosis KARI Antigens for Diagnostic Assays

The primary criteria for selection of M. tuberculosis antigens for diagnostic antigen-based assays is their presence in TB-positive sputa and immunogenicity to provide for a simply diagnostic test. The candidate antigens were identified in TB-positive sputa as described in the following paragraphs.

### a) Determination of Protein Content

The protein content of the samples was estimated using a Bradford assay. Prior to rehydration of IPG strips, samples were centrifuged at 21000×g for 10 minutes. The supernatant was collected and 10 μl of 1% Orange G (Sigma) per ml added as an indicator dye.

### b) Two-Dimensional Gel Electrophoresis

### First Dimension

Dry 11 cm IPG strips (Amersham-Biosciences) are rehydrated for 16-24 hours with 180 μl of protein sample. Rehydrated strips were focussed on a Protean IEF Cell (Bio-Rad, Hercules, Calif.) or Proteome System's IsoElectrIQ electrophoresis equipment for approx 140 kVhr at a maximum of 10 kV. Focussed strips were then equilibrated in urea/SDS/Tris-HCl/bromophenol blue buffer.

### c) Second Dimension

Equilibrated strips were inserted into loading wells of 6-15% (w/v) Tris-acetate SDS-PAGE pre-cast 10 cm×15 cm GelChips (Proteome Systems, Sydney Australia). Electrophoresis was performed at 50 mA per gel for 1.5 hours, or until the tracking dye reached the bottom of the gel. Proteins from the retentate fraction or flow-through fraction were stained using SyproRuby (Molecular Probes). Proteins from the eluate fraction were stained with silver according to the protocol of Shevchenko et al. (Anal Chem. 68(5): 850-8, 1996). Gel images were scanned after destaining using an AlphaImager System (Alpha Innotech Corp.). Gels were then stained with Coomassie G-250 to assist visualisation of protein spots in subsequent analyses.

### d) Mass Spectrometry:

Prior to mass spectrometry protein samples were prepared by in-gel tryptic digestion. Protein gel pieces were excised, destained, digested and desalted using an Xcise™, an excision/liquid handling robot (Proteome Systems, Sydney, Australia and Shimadzu-Biotech, Kyoto, Japan) in association with the Montage In-Gel Digestion Kit (developed by Tyrian Diagnostics and distributed by Millipore, Billerica, Ma, 01821, USA). Prior to spot cutting, the 2-D gel was incubated in water to maintain a constant size and prevent drying. Subsequently, the 2-D gel is placed on the Xcise, a digital image was captured and the spots to be cut are selected. After automated spot excision, gel pieces were subjected to automated liquid handling and in-gel digestion. Briefly, each spot was destained with 100 μl of 50% (v/v) acetonitrile in 100 mM ammonium bicarbonate. The gel pieces were dried by adding 100% acetonitrile, the acetonitrile was removed after 5 seconds and the gels dried completely by evaporating the residual acetonitrile at 37° C. Proteolytic digestion was performed by rehydrating the dried gel pieces with 30 μl of 50 mM ammonium bicarbonate (pH 7.8) containing 5 μg/mL modified porcine trypsin and incubated at 37° C. overnight.

Ten microliters (10 μl) of the tryptic peptide mixture was removed to a clean microtitre plate in the event that additional analysis by Liquid Chromatography (LC)—Electrospray Ionisation (ESI) MS was required.

Automated desalting and concentration of tryptic peptides prior to MALDI MS was performed using R2-based chromatography. Adsorbed peptides were eluted from the tips onto a 384-position MALDI-TOF sample target plate (Kratos, Manchester, UK or Bruker Daltronics, Germany) using 2 μl of 2 mg/ml α-cyano-4-hydroxycinnamic acid in 90% (v/v) acetonitrile and 0.085% (v/v) TFA.

Digests were analyzed using an Axima-CFR MALDI MS mass spectrometer (Kratos, Manchester, UK) in positive ion reflectron mode. A nitrogen laser with a wavelength of 337 nm was used to irradiate the sample. The spectra was acquired in automatic mode in the mass range 600 Da to 4000 Da applying a 64-point raster to each sample spot. Only spectra passing certain criteria were saved. All spectra undergo an internal two point calibration using an autodigested trypsin peak mass, m/z 842.51 Da and spiked adrenocorticotropic hormone (ACTH) peptide, m/z 2465.117 Da. Software designed by Proteome Systems, as contained in the web-based proteomic data management system BioinformatIQ™ (Proteome Systems), was used to extract isotopic peaks from MS spectra.

Protein identification was performed by matching the monoisotopic masses of the tryptic peptides (i.e. the peptide mass fingerprint) with the theoretical masses from protein databases using IonIQ or MASCOT database search software (Proteome System Limited, North Ryde, Sydney, Australia). Querying was done against the non-redundant SwissProt (Release 40) and TrEMBL (Release 20) databases (June 2002 version), and protein identities are ranked through a modification of the MOWSE scoring system. Propionamide-cysteine (cys-PAM) or carboxyamidomethyl-cysteine (cys-CAM) and oxidized methionine modifications were taken into account and a mass tolerance of 100 ppm is allowed.

Miscleavage sites were only considered after an initial search without miscleavages had been performed. The following criteria were used to evaluate the search results: the MOWSE score, the number and intensity of peptides matching the candidate protein, the coverage of the candidate protein's sequence by the matching peptides and the gel location.

In addition, or alternatively, proteins were analysed using LC-ESI-MS. Tryptic digest solutions of proteins (10 μl) were analysed by nanoflow LC/MS using an LCQ Deca Ion Trap mass spectrometer (ThermoFinnigan, San Jose, Calif.) equipped with a Surveyor LC system composed of an autosampler and pump. Peptides were separated using a PepFinder kit (Thermo-Finnigan) coupled to a C18 PicoFrit column (New Objective). Gradient elution from water containing 0.1% (v/v) formic acid (mobile phase A) to 90% (v/v) acetonitrile containing 0.1% (v/v) formic acid (mobile phase B) was performed over a 30-60-minute period. The mass spectrometer was set up to acquire three scan events one full scan (range from 400 to 2000 amu) followed by two data dependant MS/MS scans.

### e) Bioinformatic Analysis:

Following automated collection of mass spectra peaks, data were processed as follows. All spectra were firstly checked for correct calibration of peptide masses. Spectra were then processed to remove background noise including masses corresponding to trypsin peaks and matrix. The data were then searched against publicly-available SwissProt and TrEMBL databases using Tyrian Diagnostics search engine IonIQ v69 and/or MASCOT. PSD data was searched against the same databases using the in-house search engine FragmentastIQ. LC MS-MS Data are also searched against the databases using the SEQUEST search engine software.

12. Validation of M. tuberculosis Antigens and Antibodies for Diagnostic Assays

Validation of candidate diagnostic markers and antibodies was performed by determining the presence of the corresponding endogenous antigen in whole cell lysates (WCL) derived from cultures of the laboratory strain of M. tuberculosis designated H37Rv, and in whole cell lysates (WCL) derived from cultures of two M. tuberculosis clinical strains designated CSU93 and HN878, using an amplified ELISA system as described herein below. Filtrates of cell culture supernatanats harvested from whole cells were also employed. Antigens and antibodies that were detectable in all three strains were selected for further validation.

Validation of candidate diagnostic markers and antibodies was also performed by determining the specific expression of the corresponding endogenous antigen in whole cell lysates (WCL) derived from cultures of non-Mycobacterial organisms e.g., Escherichia coli, Bacillus subtilis and Pseudomonas aeruginosa, using an amplified ELISA system as described herein below. Filtrates of whole cell lysates were also employed. Antigens and antibodies that were detected specifically in M. tuberculosis were preferred.

Validation of candidate diagnostic markers and antibodies was also performed by determining the specific expression of the corresponding endogenous antigen in whole cell lysates (WCL) derived from cultures of the laboratory strain of M. tuberculosis designated H37Rv, as opposed to expression in other Mycobacteria species e.g., M. avium and M. intracellulaire, using an amplified ELISA system as described herein below. Filtrates of supernatants from whole cell cultures were also employed. Antigens that were detected specifically by particular antibodies in M. tuberculosis were preferred, however those that were also expressed in M. avium and/or M. intracellulaire were not discarded. This is because a diagnostic test that tests any Mycobacteria in a sample has utility as a primary generic assay and can be employed in conjunction with species-specific tests for M. tuberculosis e.g., employing specific diagnostic markers as disclosed herein and/or a culture test to determine the presence or absence of M. tuberculosis.

### 13. Preparation of Whole Cell Lysates

Proteins were extracted from lyophilised M. tuberculosis cells. The cells were resuspended in an extraction buffer and processed in a bead mill to rupture the cells and release the proteins. The cell debris was pelleted by centrifugation and the supernatant used as the whole cell lysate (WCL). A Bradford colorimetric assay was done to estimate the protein concentrations. In some instances, a cytosolic extract obtained from Colorado State University was used.

### 14. Antibody Production Methods

Antibodies were prepared by immunization with a synthetic immunogenic peptide derived from a specific immunogenic protein of M. tuberculosis identified as described in the subsequent examples, or alternatively, by immunization with the full-length immunogenic protein of M. tuberculosis or an immunogenic fragment of the M. tuberculosis immunogenic protein produced by recombinant means using standard procedures. For recombinant protein or fragment production, a DNA sequence of M. tuberculosis strain H37Rv that encodes the immunogen was isolated and cloned into a suitable vector for expression in Escherichia coli, and the expressed protein or fragment was purified by standard chromatography techniques.

### 15. Antibody Selection Criteria

Antibodies were selected based on their sensitivity and specificity towards the immunogen in ELISA with a preferred limit of detection (LOD) of e.g., less than about 100 ng/mL recombinant antigen in a single-site ELISA and/or less than about 500 pg/mL antigen in an amplified two-site ELISA. Antibodies were also selected that detected the M. tuberculosis antigen in M. tuberculosis culture with little or no cross-reactivity to other Mycobacteria species or non-Mycobacteria pathogens.

Antibodies were screened initially by reactivity against the immunogen in each case using one-site ELISA. The skilled artisan will be aware that one-site ELISA requires unlabelled recombinant immunogen bound to the surface of a solid substrate and a labelled detector antibody e.g., an antibody conjugated to a detectable marker such as colloidal gold or biotin, wherein the detector antibody specifically binds to an epitope on a target antigen contained in the immobilized immunogen. The detector antibody binds to the immobilized immunogen such that it is immobilized on the solid substrate and labelled indirectly by binding of the label on the detector antibody.

Alternatively, or in addition a two-site ELISA was performed, subject to availability of antibodies with which to pair the test antibody in a two-site test. Two-site ELISA requires an unlabelled capture antibody bound to the surface of a solid substrate and a labelled detector antibody e.g., an antibody conjugated to a detectable marker such as colloidal gold or biotin, wherein both the capture antibody and detector antibody specifically bind to a target antigen albeit to different or non-interfering epitopes on the target antigen. When the antigen is present in a test sample, the detector antibody and capture antibody “sandwich” the antigen such that it is immobilized on the solid substrate and labelled indirectly by binding of the label on the detector antibody.

Generally, for antibodies having a LOD in two-site ELISA of less than about 500 pg/mL, Western blot (WB) immune-electrophoresis was performed to confirm the specificity of antibodies for the recombinant protein and an endogenous M. tuberculosis protein of the expected molecular weight that is present in whole cell lysates. Briefly, recombinant proteins and whole cell lysates were separated by electrophoresis on 1-dimensional SDS/polyacrylamide gels (10% polyacrylamide Nu-PAGE gels) using a MOPS or MES buffer system according to the manufacturer's instructions. The resolved proteins were transferred from the gels onto PVDF membranes using a semi-dry electro blotting system. The membranes were then probed with primary antibodies to the corresponding antigens followed by HRP-conjugated secondary antibodies capable of binding the antibody probe used according to standard procedures. Specific signals were then visualized by a chemiluminescence detection system and an image acquired using either X-ray film followed by scanning or using the Fuji-LAS-3000 imager to acquire an image directly from the treated blot. For each antigen tested, Western blot conditions were optimized in terms of dilution of the primary and secondary antibodies required to provide optimum signal to noise ratio (data not shown). Replica blots were probed with primary and secondary antibodies (positive control) or secondary antibody alone (negative control).

These Western data were confirmed by competition experiments wherein a primary antibody was pre-incubated with a molar excess of recombinant immunogen in solution prior to conducting the Western blot to thereby compete for epitopes on the resolved proteins. Thus, loss of signal in Western blotting confirms an initial conclusion of antibody specificity. Briefly, Western Blot analysis was performed as described in the preceding paragraph, except that primary antibody was pre-incubated with a 100- to 200-molar excess of the corresponding recombinant protein. Blots were probed in the usual manner.

### 16. Selection of Antibody Pairs

For selection of antibody pairs, two-site ELISA was performed using the most sensitive antibodies available that meet the antibody selection criteria described in the preceding section. In performing two-site ELISA in the context of selecting antibody pairs, the inventors employed antibody candidates in both configurations as detector and capture antibody, to thereby determine optimum configurations of capture and detector antibodies. In general, detector antibody was employed at different dilutions against a titration of recombinant immunogen for each capture antibody concentration tested. Preferred detector antibodies for this purpose are biotinylated antibodies that were detectable using poly-HRP-conjugated streptavidin. When biotinylated detector antibody was not available, a preferred detector antibody comprises an unlabelled detector antibody that is detectable by sequential binding of (i) biotinylated secondary antibody (e.g., anti-rabbit Ig or anti-chicken Ig or anti-mouse Ig) to the detector antibody and (ii) poly-HRP-conjugated streptavidin to the bound biotinylated secondary antibody.

### 17. ELISA Formats

### a) One-Site ELISA

A NUNC plate was coated with a serial dilution of the recombinant protein and incubated overnight at 4° C. After blocking, the plate was incubated with various dilutions of the test antibody followed by HRP-conjugated secondary antibody and TMB. The volume of each reaction was 50 μl. The plate was washed between each addition. The immune reaction was stopped by the addition of 0.5 M H₂SO₄ after an appropriate time based on visual examination of color development (usually about 30 min), and the OD read in a microplate reader at wavelengths of 450 nm and 620 nm.

The resulting data were exported to Microsoft Excel where the Delta OD (OD 450-620), referred to as OD, was recorded for data analysis. The sensitivity of the assay was determined as described below and expressed as LODAbT. An antibody with an LODAbT of less than about 100 ng/mL was further tested for suitability as either a capture or detector antibody in a sandwich ELISA.

### b) Standard Two-Site or “Sandwich” ELISA

A standard sandwich ELISA was performed using selected antibody pairs e.g., to determine their suitability as either capture or detector antibodies. NUNC immune-plates were coated with various dilutions of capture antibody, then incubated sequentially with the relevant recombinant protein or whole cell lysate of filtrate comprising a test immunogen, and various dilutions of detector antibody, HRP-conjugated secondary antibody and SIGMA TMB. The volume of each reaction was 50 μl. The plate was washed between each addition. The immune reaction was stopped by the addition of 0.5 M H₂SO₄ after an appropriate time based on visual examination of color development (usually about 30 min), and the OD read in a microplate reader at wavelengths of 450 nm and 620 nm.

The resulting data were exported to Microsoft Excel for analysis. The sensitivity of the assay was determined as described below and referred to as the LOD; antibody pairs producing the lowest LOD scores (e.g., less than about 3 ng/mL) were selected for further optimization in an amplified sandwich ELISA.

### c) Amplified Sandwich ELISA

Amplified sandwich ELISA is performed as for standard sandwich ELISA as described herein above and analysed by the same procedures, except plates were incubated with either a biotinylated detector antibody or a detector antibody followed by a biotinylated secondary antibody. Amplification is achieved by the addition of, 50-200 μl of various dilutions of poly80-HRP-streptavidin then, 50-200 μl of Pierce TMB. Antibody pairs producing the lowest LOD scores (e.g., less than about 500 pg/mL) were selected.

### d) ELISA Data Analysis

ELISA data were exported to Microsoft Excel for analysis. A standard curve was plotted using an X-Y graph with the mean OD+SD (OD=OD_{450 nm}−OD_{620 nm}) on the Y axis and the recombinant protein/peptide concentration (e.g., pg/mL) on the X axis (logarithmic scale). The coefficient of variation, calculated as the standard deviation divided by the mean and expressed as a percent (CV %), was used as a measure of intra-assay and inter-assay variability.

GraphPad Prism software was employed to fit a 4-parameter logistic curve to the standard curve data points. Antigen concentrations for unknown samples were determined by interpolation of the OD values off the generated curve.

### e) Determination of Limit of Detection (LOD Values

The Limit of Detection (LOD) for an antibody pair in a two-site “sandwich” ELISA where a suitable calibration curve was available was defined as the concentration of immunogen that produced an OD value equivalent to the average baseline value plus 3×SD, using functions available in GraphPad Prism software. For one-site ELISA, or for sandwich ELISA where calibration curves were not available, the LOD of the antibody or antibody pair was estimated using Microsoft Excel. For each immunogenic protein being analyzed, the optical density data from ELISA dose response curves were used to calculate LOD values.

### For One-Site ELISA:

For one-site ELISA data or two-site ELISA data where there were insufficient data points for software to apply a non-linear regression curve fit function, an estimate of the LOD was obtained in Excel. The start of the baseline of the dose response curve and the baseline mean OD+3×SD were determined as follows: Increments in optical density, calculated as the difference between optical density for a given immunogen concentration and the optical density for the next higher immunogen concentration, were determined. The data point where the increment in optical density was less than about 0.05 OD units was deemed to be the start of the baseline. Average and SD optical density values for replicate samples at the deemed baseline start point and at the next two incremental points were used to calculate the average+3×SD for the series. The concentration of immunogen producing an optical density greater than the mean baseline OD+3×SD was deemed the LOD value.

### For Sandwich ELISA:

In one approach, the concentration of recombinant protein/peptide immunogen i.e., the value “log₁₀ [immunogen]” and the replicate optical density values obtained from sandwich ELISA were transferred into an Excel worksheet template that has been generated to automatically calculate the necessary values from the raw ELISA data. An R² value was generated as an estimate of the goodness of fit of the standard curve, and a value greater than about 0.99 was accepted as a good fit. The 99% Confidence Interval (CI) value ranges were also calculated. The maximum value in the range for the bottom asymptote of the fitted curve was interpolated from the fitted standard curve. The interpolated value, when un-logged, represented the recombinant protein concentration having an OD value equivalent to the average baseline value plus 3×SD. That value, referred to as the LOD value, was deemed to indicate sensitivity of the ELISA.

Alternatively, the log₁₀ [immunogen] and the corresponding replicate optical density values were exported into GraphPad Prism where a non-linear regression curve fit function was used to fit a 4-parameter logistic curve to the data points. A non-linear regression curve fit function was used to fit a sigmoid curve to the data. An R² value was generated as an estimate of the goodness of fit of the standard curve, and a value greater than about 0.99 was accepted as a good fit. The concentration of recombinant protein/peptide immunogen corresponding to the baseline mean optical density (OD) plus 3×SD was interpolated from the standard curve.

### Example 2

### Detection of Active TB Complex in Clinical Sputa Using Quantitative PCR

In the first set of experiments the inventors sought to develop a quantitative real-time PCR assay for the detection of ilvC biomarker (SEQ ID NO:2) encoding Mycobacterium tuberculosis complex KARI protein (SEQ ID NO:1), as a means of detection of tuberculosis or an infection by one or more organisms of the Mycobacterium tuberculosis complex. The inventors also sought to correlate the results obtained with severity of infection as demonstrated by smear status grading and to compare the sensitivity of the assay for the detection of ilvC biomarker with detection of other biomarkers of one or more organisms of the Mycobacterium tuberculosis complex. Other biomarkers assayed included nucleic acids (SEQ ID NOs: 4, 6, 8 and 28) encoding, Mycobacterium tuberculosis BSX protein (SEQ ID NO:3), Mycobacterium tuberculosis Rv1265 protein (SEQ ID NO:5) and Mycobacterium tuberculosis S9 protein (SEQ ID NO:7). The 16S rRNA marker (SEQ ID NO:27) was also included. Specific primers for the targets of interest were designed as described herein, and are listed in Table 1.

The 16S rRNA gene was chosen as a housekeeping gene. The 16S rRNA primers were not specific to are not specific to organisms of the Mycobacterium tuberculosis complex and may detect several other Mycobacterium sp. However, specificity of the ilvC primers were analyzed, which showed specificity for the M.tb complex amongst the mycobacterial genre. The primers and the 126 base pair amplified product were then screened to ensure specificity by performing a BLAST search across the 940 bacterial, 48 achaeal, and 162 eukaryotic genomes trees available at NCBI. The search was performed and database information was recorded. These data (not shown) indicate 100% homology across the M.tb. complex, with no other mycobacteria showing significant homology. These data confirm the primers were specific for mycobacteria of the M.tb complex and not mycobacteria or other bacteria per se. A similar search on the human genome database gave no hits.

TABLE 1
depicting primers used in this study

| SEQ | | | |
|---|---|---|---|
| ID No. | Primer Name | Sequence (5′-3′) | Target |
| 17 | rtM.tb16SF | CGTTCCCGGGCCTTGTAC | 16S rRNA |
| 18 | rtM.tb16SR | CGGGTGTTACCGACTTTCATG | |
| 19 | rtM.tbilvCF | GGCAACAAACAGCTCGAAGAG | ilvC |
| 20 | rtM.tbilvCR | CATCAGGTCGCGGAGTTTCT | |
| 21 | rtM.tbBSXF | ACCAAGGCCTCCGATGTG | BSX |
| 22 | rtM.tbBSXR | AGACGTGCGCCGTCTCA | |
| 23 | rtM.tbRv1265F | CGAATGACTATCGCCGATGAA | Rv1265 |
| 24 | rtM.tbRv1265R | GCCATGTGCGTGAACAAGAT | |
| 25 | rtM.tbS9F | CACAATCGTTCGTGTTGGAG | S9 |
| 26 | rtM.tbS9R | GTGCACCTTGTTTGGGAAGT | |

### 1. Standard Curves

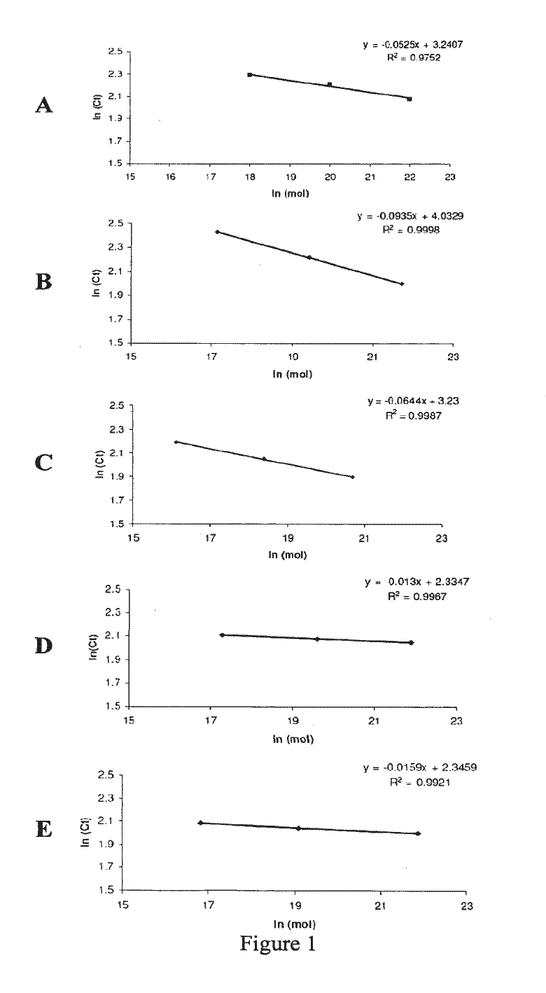

Standard curves for each primer set described in Table 1, were calculated using M. tuberculosis genomic DNA as PCR template as described in Example 1, and are provided in FIG. 1. The standard curves shown in FIG. 1 were used to calculate the amount of transcript copies per μg of cDNA. The curves were linear up to 9.7×10⁹ transcript copies (approximately 1 μg target DNA).

### 2. Quantitative Real Time PCR Assay and Smear Status Grading

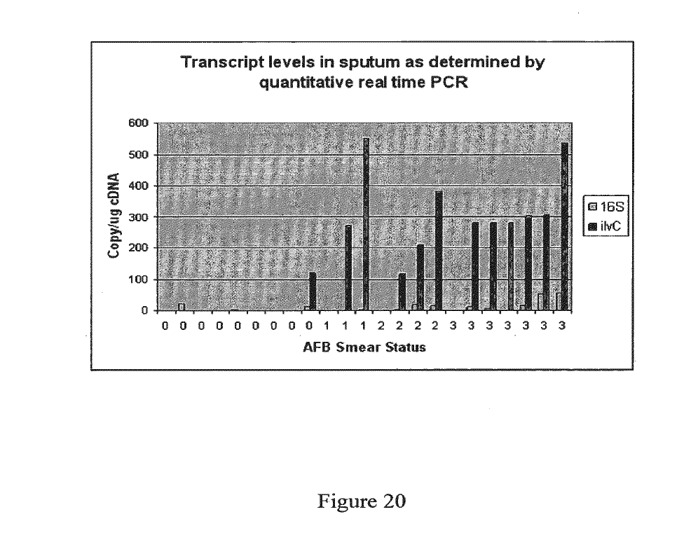

Using the designed primers, a cohort of samples was screened across 2 clinical populations, i.e., Cameroon and USA. Thirteen (13) TB-negative and TB-positive sputa samples collected from subjects as described in Example 1 were used to evaluate nucleic acid based assay using primer pairs. Total RNA was extracted from the samples and cDNA was synthesized and used in quantitative real-time PCR as described in Example 1. The sputa samples were also subjected to smear status grading according to conventional methods accepted in the art, as described in Example 1. The results obtained for sputum samples using real time PCR for ilvC and 16S rRNA and smear grading results for 13 samples are presented in Table 2 below and FIG. 20.

TABLE 2
Transcript levels of ilvC and 16S rRNA as determined
by quantitative real-time PCR.

| Sample ID | Sample Source | Smear Status | 16S | ilvC |
|---|---|---|---|---|
| 359 | Cameroon | 3+ | 55.91 | 535.96 |
| 344 | Cameroon | 2+ | 14.07 | 380.84 |
| 345 | Cameroon | 2+ | 17.22 | 210 |
| 338 | Cameroon | 2+ | 3.1 | 118.2 |
| T114 | Thailand | 1+ | 360 | 93.6 |
| SF98-2005 | BD | neg | 0.38 | 137.5 |
| SF98-2140 | BD | neg | 0.98 | nd |
| 56158 | BD | neg | 3.75 | nd |
| 49998 | BD | neg | 904 | nd |
| 364 | Cameroon | neg | 0.06 | nd |
| 116 | Australia | neg | 23.4 | nd |
| 49916 | BD | neg | 3.17 × 10² | nd |
| 49847 | BD | neg | 2.84 × 10⁴ | nd |

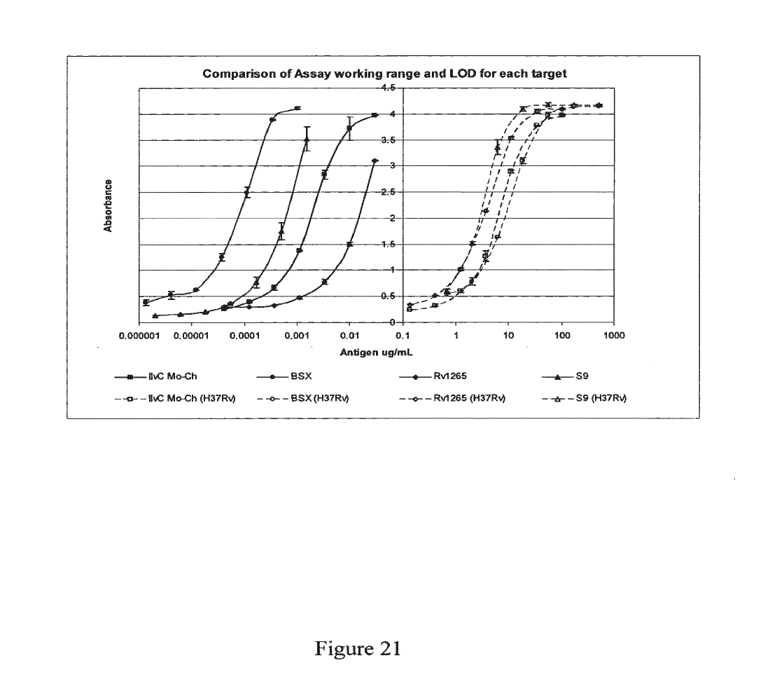

Values are expressed as transcript copy/μg cDNA. ND represents that the target ilvC or 16S rRNA transcript was not detected in the sputum sample, when the highest amount of total RNA (500 ng) extracted from the sputum sample was used for cDNA synthesis. Also shown are smear status grading for each sample, ranged from negative (neg) to 3+ as correlated with severity of infection. In this cohort, the data show sensitivity of 100%, and 88% specificity for ilvC transcript detection. Furthermore, the level of ilvC expression correlated with smear grading. Further samples were analyzed within this cohort. The level of ilvC expression along with the expression of 16rRNA was determined and plotted vs smear status as shown in FIG. 21. These data demonstrate that detection of ilvC nucleic acid is 15-20× more sensitive than 16S rRNA detection by PCR, in this example. Moreover, the data indicate that ilvC transcript remains relatively stable in clinical samples.

3. Real-Time PCR of M. tuberculosis Complex Nucleic Acids in Clinical Samples

Three additional samples ('Spike', ‘116 spike’ and ‘4436 spike’) were then included in the analysis to which M. tuberculosis cells were added, and RNA extraction, cDNA synthesis and real-time PCR was performed as described above. All samples were assayed by real time PCR for ilvC and 16S rRNA, BSX, S9 and Rv1265. The results obtained are shown in Table 3 below.

Comparison of the detection of the ilvC, 16S rRNA, BSX, S9, Rv1265 by real-time PCR assay showed that detection of ilvC transcript was most reliable despite the transcript levels being exceptionally low (0.01 copies/cell for the M. tuberculosis culture, compared to 268 copies/cell of 16S transcript). In contrast, the signals for the other three biomarkers (BSX, S9 and Rv1265) were too low to permit quantitation of those transcripts in sputa when the highest possible amount of nucleic acid was used in the reactions. The inability of the current RT-PCR assay to detect BSX, S9 and Rv1265 in sputa may be due to a relatively low abundance of the respective transcripts, and/or sample degradation due to the action of RNAses in the sample and a high amount of contaminating host RNA.

To estimate the presence of inhibitors in the sputum, which could have interfered with the RNA extraction and cDNA synthesis steps, all samples were spiked with a culture suspension of M.tb. Several samples (49847, thai143, mpc379, mpc359) were found to have a lower number of 16S transcripts when spiked, suggesting that inhibition prevented accurate quantification. The main difficulty when interpreting the results from the spiking is the unknown contribution of contaminating RNA (especially host cells) in the sample, and is the reason for the highly variable levels in 16S.

The results obtained from the RT-PCR assay for the reference 16S gene and ilvC are presented in Table 3. It should be noted that the 16S is not M.tb-specific or M.tb-complex specific, but detects several other Mycobacterium spp., including M. avium, M. avium subsp. paratuberculosis and M. intracellulare. Therefore, the 16S rRNA gene was detected for all samples, regardless of the reported smear result.

Values are expressed as transcript copy/μg cDNA. ND represents that the target ilvC 16S rRNA, BSX, S9, or Rv1265 transcript was not detected in the sputum sample, when the highest amount of total RNA (500 ng) extracted from the sputum sample was used for cDNA synthesis.

Based on the results provided herein, the inventors provide a nucleic acid amplification based assay e.g., using real time PCR, for the detection of the M.tb complex in clinical samples which can be correlated with infection by one or more organisms of the M.tb. complex and which is based on the detection of ilvC target transcript nucleic acids. The inventors have demonstrated herein that ilvC provides a advantageous target biomarker of infection by one or more organisms of the M.tb complex, due to high abundance of ilvC transcript copies in subject sample, for example sputum. Amplification of RNA transcripts for the other target genes of the M.tb. complex e.g., BSX, S9, Rv1265, may be difficult to detect possible due to low abundance of transcript copy number of those genes in cells of the infecting organism.

TABLE 3
depicting transcript levels of ilvC, 16S rRNA, BSX, S9, Rv1265
as determined by quantitative real-time PCR.

| Sample | 16S | ilvC | BSX | S9 | Rv1265 |
|---|---|---|---|---|---|
| mpc344 | 14.07 | 380.84 | 14.9 | nd | nd |
| mpc345 | 17.22 | 210 | nd | nd | nd |
| mpc348 | 3.1 | 118.2 | nd | nd | nd |
| T114 | 360 | 93.6 | nd | nd | nd |
| SF98-2005 | 0.38 | 137.5 | nd | nd | nd |
| SF98-2140 | 0.98 | nd | nd | nd | nd |
| 56158 | 3.75 | nd | nd | nd | nd |
| BD49998 | 904 | nd | nd | nd | nd |
| Spike | 4 × 10⁶ | 1.23 × 10³ | 0.42 | nd | nd |
| 359 | 55.91 | 535.96 | nd | nd | nd |
| 364 | 0.06 | nd | nd | nd | nd |
| 116 | 23.4 | nd | nd | nd | nd |
| 116 Spike | 2.69 × 10⁵ | 1.96 × 10³ | nd | nd | nd |
| 49916 | 3.17 × 10² | nd | nd | nd | nd |
| 4436 spike | 5.2 × 10⁴ | 4.85 × 10⁵ | nd | nd | nd |
| 49847 | 2.84 × 10⁴ | nd | nd | nd | nd |

Notwithstanding that detection of amplified nucleic acids encoding BSX, S9 and Rv1265 may be more difficult to detect that amplification of ilvC nucleic acids in M.tb complex, the ilvC amplification described herein may be employed simultaneously or sequentially with one or more sets of primers against nucleic acids encoding M. tuberculosis Rv1265 and/or M. tuberculosis BSX, and/or M. tuberculosis S9 and/or M. tuberculosis as described herein and/or EF-Tu protein and/or M. tuberculosis P5CR protein and/or M. tuberculosis TetR-like protein and/or glutamine synthetase protein and combinations thereof, which have low cross reactivity to other Mycobacteria tested. In interpreting such a multi analyte test, detection of ilvC nucleic acids indicates the presence of M.tb complex in the clinical sample and the additional detection of nucleic acids encoding M. tuberculosis Rv1265 and/or M. tuberculosis BSX, and/or M. tuberculosis S9 and/or M. tuberculosis and/or EF-Tu protein and/or M. tuberculosis P5CR protein and/or M. tuberculosis TetR-like protein and/or glutamine synthetase protein and combinations thereof indicates a greater likelihood of M. tuberculosis or M.tb complex infection.

Alternatively, or in addition, ilvC-NAA described herein, may be employed simultaneously or sequentially with antigen based assay using antibodies against KARI protein encoded by ilvC as described herein in examples 3 et seq. Alternatively, or in addition, such assays may also be employed simultaneously or sequentially with a ligand-based assay using an isolated KARI protein or fragment thereof encoded by ilvC or fragment thereof to detect a ligand or an antibody again KARI in clinical sample, as described herein.

In summary, the data herein indicate that the qRT-PCR assay is able to detect M.tb RNA or M.tb complex RNA in all sputum samples tested, however ilvC is the preferred biomarker best applicable to quantification by RT-PCR due to its transcript stability and, variability in the assay may arise from the presence of inhibitory compounds e.g., non-M.tb complex host and commensal bacterial RNAs in a low proportion e.g., about 7%, of the samples tested as well as RNA degradation.

### Example 3

### Antigen-Based Diagnosis of Tuberculosis or Infection by M. tuberculosis Using Antibodies that Bind to M. tuberculosis Ketol-Acid Reductoisomerase (KARI)

### 1. Identification of KARI Protein in TB-Positive Subjects

A protein having a molecular weight of about 36 kDa was recognized in TB+ samples. The sequences of ten peptides from MALDI-TOF data matched a sequence encoded by the ilvC gene of M. tuberculosis set forth in SEQ ID NO: 1. The percent coverage of SEQ ID NO: 1 by these 10 peptides was about 37%, suggesting that the peptide fragments were derived from this same protein marker.

The identified protein having the amino acid sequence set forth in SEQ ID NO: 1 is a putative Ketol-Acid Reducto Isomerase and was designated as “KARI”.

### 2. Antibodies

Antibodies were prepared against recombinant KARI protein encoded by the ilvC gene of M. tuberculosis (SEQ ID NO:2) using procedures described herein. Ten (10) antibodies were produced and screened for their suitability as described in Example 1. This process led to the identification of an antibody pair for diagnosis of M. tuberculosis consisting of a mouse-derived antibody designated “Mo1283F” as a preferred capture antibody and a chicken derived polyclonal antibody designated “Ch34/35” as a preferred detector antibody. Other orientations and antibody combinations are not excluded.

### 3. Validation of KARI and Antibodies Thereto as Diagnostic Reagents

The amino acid sequence of KARI protein from M. tuberculosis strain H37Rv is presented as SEQ ID NO: 1. The translation product has an expected molecular mass of about 36 kDa. One-dimensional SD S/PAGE analysis of a hexa-histidine-tagged rKARI protein performed essentially as described in Example 1 showed that the KARI protein migrated as a single band of approximately 37 kDa (data not shown), which is the expected mass of the fusion protein, based on the theoretical mass of the translation product and the hexahistidine tag moiety.

Western blot analyses were carried out using ELISA capture (Mo1283F) and detector (Ch34/35) antibodies separately, to detect recombinant KARI protein and endogenous KARI protein in whole cell lysates of M. tuberculosis H37Rv, M. tuberculosis CSU93 and M. tuberculosis HN878. Both antibodies recognized a band in whole cell lysates derived from all three M. tuberculosis strains that had the expected molecule mass of native KARI protein (i.e., about 36 kDa), as well as detecting the slightly larger recombinant KARI protein (data not shown). Binding was highly-specific with little background. The available data therefore confirm the specificity of the antibodies Mo1283F and Ch34/35 for detecting the M. tuberculosis KARI protein.

Competition Western blot analysis performed essentially as described in Example 1 indicated that binding of the polyclonal antibody Ch34/35 to recombinant KARI protein and endogenous KARI protein could be ablated by pre-incubation of antibodies with excess concentration of unlabelled recombinant KARI protein (data not shown).

In summary, the available data indicate that the antibodies Mo1283F and Ch34/35 antibodies bind to M. tuberculosis KARI protein specifically.

4. Amplified Sandwich ELISA for Detection of M. tuberculosis KARI Protein

Amplified ELISA was performed essentially as described in this example and in Example 1, using 5 μg/mL of Mo1283F antibody as a capture reagent and 2.5 μg/mL of Ch34/35 polyclonal antibody as a detector antibody, and a biotinylated secondary antibody with HRP-conjugated streptavidin to detect the bound detector antibody.

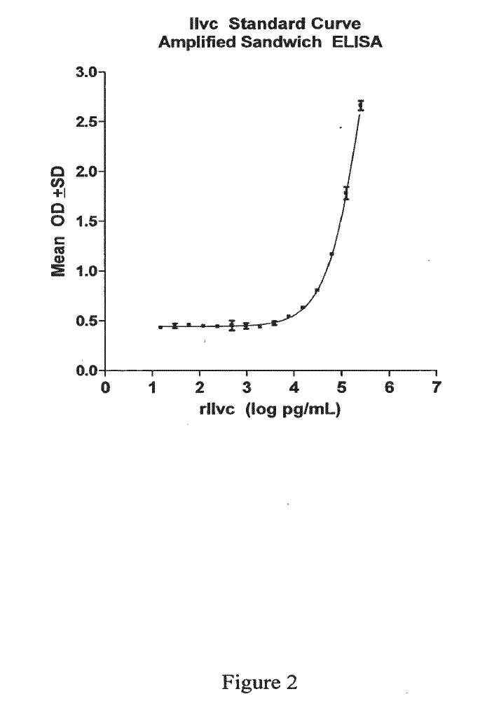

Data presented in FIG. 2 indicate that under the assay conditions tested and with this preferred, albeit not essential, orientation of antibodies, there is low background noise and a LOD of about 1690 pg/mL. Such sensitivity of detection coupled with low background in sandwich ELISA is considered by the inventors to be within useful limits.

5. Cross-Reactivity Between Anti-Kari Antibodies and Different M. tuberculosis Isolates

To further assess the suitability of KARI as a diagnostic marker for the presence of M. tuberculosis in biological samples, and to assess the specificities of antibodies prepared against KARI protein, the inventors compared antibody reactivities in amplified sandwich ELISA performed as described herein above between cellular extracts of the clinical M. tuberculosis strains CSU93 and H878 and the laboratory M. tuberculosis strain H37Rv.

Briefly, an ELISA plate was coated overnight with capture antibody Mo1283F. Following washing to remove unbound antibody, a cellular extract from each isolate was added to the wells of the antibody-coated ELISA plates. As a negative control for each assay, buffer without cellular extract was used. Following incubation for 1 hour and washing to remove unbound antigen, detection antibody Ch34/35 was contacted with the bound antigen-body complexes. Following incubation at room temperature for 1 hour, plates were washed, incubated with 50 μl of diluted secondary antibody (e.g., biotinylated donkey anti-chicken IgG and poly-40 streptavidin-HRP conjugate) for 1 hour, washed again, incubated with TMB for 10 mins, and the absorbance at 450-620 nm was determined. Samples were assayed in duplicate over three dilutions of whole cell extracts. A calibration standard curve was produced based on standardized levels of KARI protein.

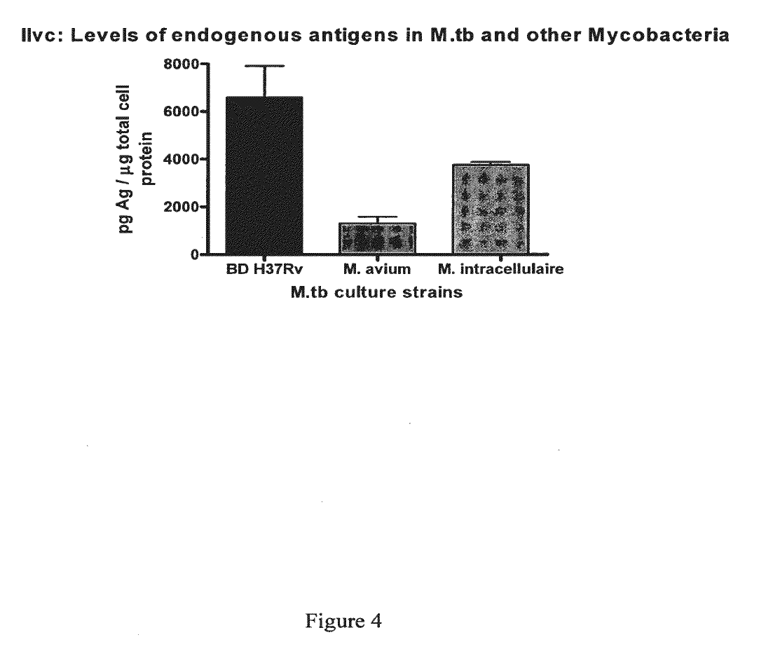

Data presented in FIG. 4 show that M. tuberculosis KARI protein is present in both the clinical M. tuberculosis isolate CSU93 and the laboratory strain H37Rv at comparable levels. Lower levels of KARI protein were detectable in M. tuberculosis H878, suggesting that antibodies against KARI protein may not distinguish specific M. tuberculosis clinical strains.

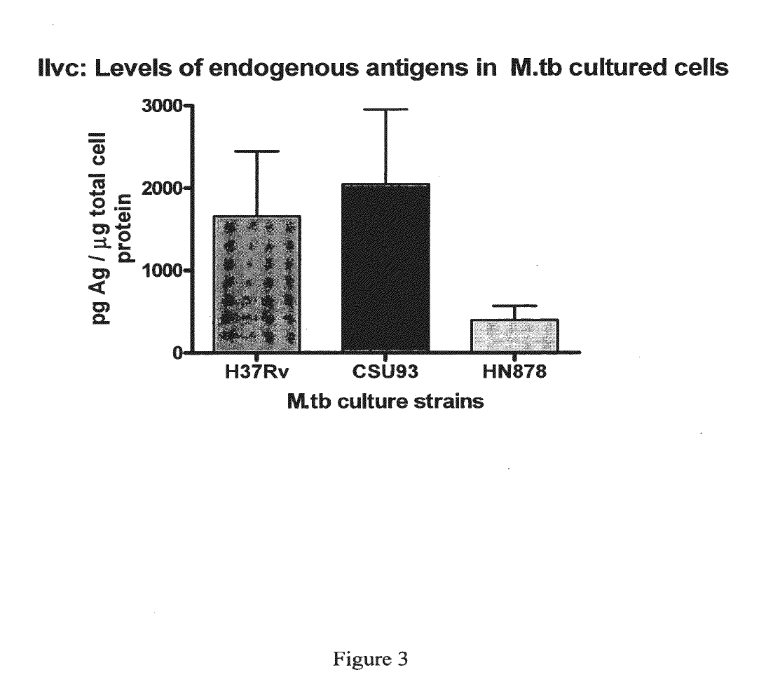

The data presented in FIG. 3 do not abrogate utility of antibodies against KARI protein in a general single-analyte diagnostic test, or alternatively, as part of a multi-analyte test in conjunction with antibodies against specific strains of M. tuberculosis such as described herein or known in the art.

For example, antibodies against KARI protein may be employed in conjunction with subsequent culture of M. tuberculosis from KARI-positive clinical specimens to yield information on clinically-relevant strains present in the sample, if required.

### 6. Cross-Reactivity Between Different Mycobacteria Species

To further assess the suitability of KARI as a diagnostic marker for the presence of M. tuberculosis in biological samples, and to assess the specificities of antibodies prepared against KARI protein, the inventors compared antibody reactivities in amplified sandwich ELISA performed as described herein above between cellular extracts of the Mycobacteria species M. tuberculosis, M. avium and M. intracellulaire.

Briefly, an ELISA plate was coated overnight with capture antibody Mo1283F. Following washing to remove unbound antibody, a cellular extract from each Mycobacteria species was added to the wells of the antibody-coated ELISA plates. As a negative control for each assay, buffer without cellular extract was used. Following incubation for 1 hour and washing to remove unbound antigen, detection antibody Ch34/35 was contacted with the bound antigen-body complexes. Following incubation at room temperature for 1 hour, plates were washed, incubated with 50 μl of diluted secondary antibody (e.g., biotinylated donkey anti-chicken IgG and poly-40 streptavidin-HRP conjugate) for 1 hour, washed again, incubated with TMB for 10 mins, and the absorbance at 450-620 nm was determined. Samples were assayed in duplicate over three dilutions of whole cell extracts. A calibration standard curve was produced based on standardized levels of KARI protein.

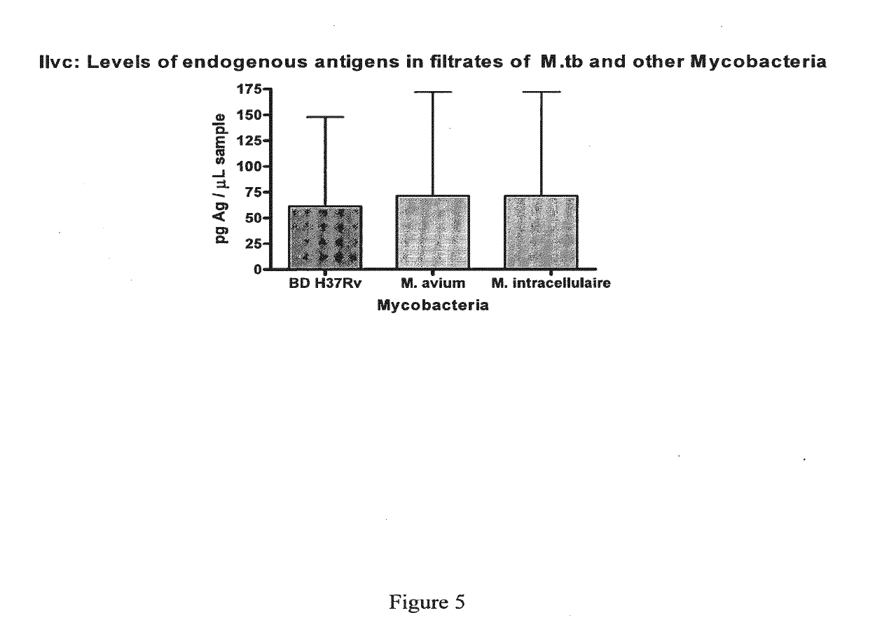

Data presented in FIGS. 4 and 5 show detectable cross-reactivity between the three Mycobacteria species, indicating that M. tuberculosis KARI protein is less suited for species-specific detection of M. tuberculosis under these assay conditions or using the selected antibody pair. This does not abrogate utility of antibodies against KARI protein in a general single-analyte diagnostic test, or alternatively, as part of a multi-analyte test in conjunction with antibodies against a species-specific marker of M. tuberculosis such as described herein or known in the art.

For example, antibodies against KARI protein may be employed in conjunction with subsequent culture of M. tuberculosis from KARI-positive clinical specimens.

Alternatively, or in addition, antibodies against KARI protein may be employed simultaneously with one or more antibodies against M. tuberculosis Rv1265 and/or M. tuberculosis BSX protein and/or M. tuberculosis EF-Tu and/or M. tuberculosis S9 protein as described herein which have low cross-reactivity to the other Mycobacteria tested. In interpreting such a multi-analyte test, binding of antibodies against KARI protein indicates the presence of a Mycobacterium in the clinical sample and the additional binding of antibodies against M. tuberculosis Rv1265 and/or M. tuberculosis BSX protein and/or M. tuberculosis EF-Tu and/or M. tuberculosis S9 protein indicates a greater likelihood of M. tuberculosis infection. The combination of antibodies against M. tuberculosis KARI protein and one or more of antibodies against M. tuberculosis Rv1265 protein and antibodies against M. tuberculosis BSX protein is especially preferred for such applications, based on the low cross-reactivity of the antibodies against Rv1265 and BSX to M. avium and M. intracellulaire.

7. Low Cross-Reactivity Between M. tuberculosis and Non-Mycobacteria Pathogens

To further assess the suitability of KARI as a diagnostic marker for the presence of M. tuberculosis in biological samples, the inventors compared antibody cross-reactivities in amplified sandwich ELISA performed between cellular extracts of M. tuberculosis strain H37Rv (a laboratory strain), Escherichia coli, Bacillus subtilis or Pseudomonas aeruginosa.

Briefly, an ELISA plate was coated overnight with capture antibody Mo1283F. Following washing to remove unbound antibody, a cellular extract from each microorganism was added the wells of the antibody-coated ELISA plates. As a negative control for each assay, buffer without cellular extract was used. Following incubation for 1 hour and washing to remove unbound antigen, detection antibody Ch34/35 was contacted with the bound antigen-body complexes. Following incubation at room temperature for 1 hour, plates were washed, incubated with 50 μl of a secondary antibody (i.e., biotinylated donkey anti-chicken IgG and poly-40 streptavidin-HRP conjugate) for 1 hour, washed again, incubated with TMB for 10 mins, and the absorbance at 450-620 nm was determined.

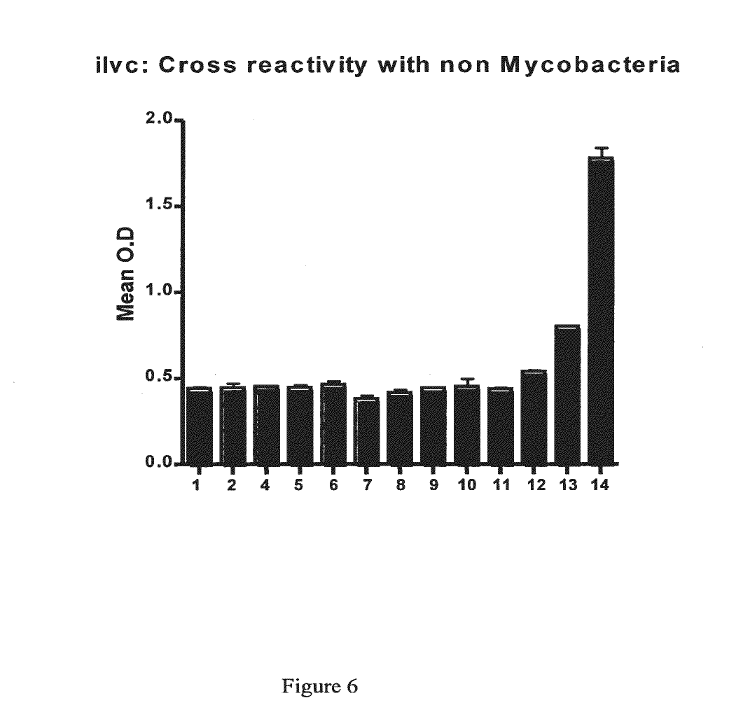

Data presented in FIG. 6 show no significant cross-reactivity of antibodies against M. tuberculosis KARI protein with Escherichia coli, Bacillus subtilis or Pseudomonas aeruginosa cellular extracts under the conditions tested, indicating that the antibodies form the basis of a Mycobacterium-specific test.

### 8. Detection of KARI Protein in Clinical Samples

To further assess the suitability of KARI as a diagnostic marker for the presence of M. tuberculosis in biological samples, the inventors determined the ability of antibodies to detect endogenous KARI protein in clinical samples obtained from TB-positive subjects who had been diagnosed previously on the basis of smear test and M. tuberculosis culture assay results. Patients had been categorized on the basis of both smear and culture test results, and HIV status. All subjects tested were both smear-negative and culture-negative or alternatively, both smear-positive and culture-positive.

Briefly, sandwich ELISA was performed as described herein above on the sputum samples, which were prepared by Method 3 and assayed as 17×150 microlitre aliquots under the replacement amplification protocol (see below). ELISA plate was coated overnight with capture antibody Mo1283F. Following washing to remove unbound antibody, treated sputa were added to the wells of the antibody-coated ELISA plates. As a negative control for each assay, buffer was used. Following incubation for 1 hour and washing to remove unbound antigen, detection antibody Ch34/35 was contacted with the bound antigen-body complexes. Following incubation at room temperature for 1 hour, plates were washed, incubated with 50 μl of a secondary antibody (i.e., biotinylated donkey anti-chicken IgG and poly-40 streptavidin-HRP conjugate) for 1 hour, washed again, incubated with TMB for 10 mins, and the absorbance at 450-620 nm was determined.

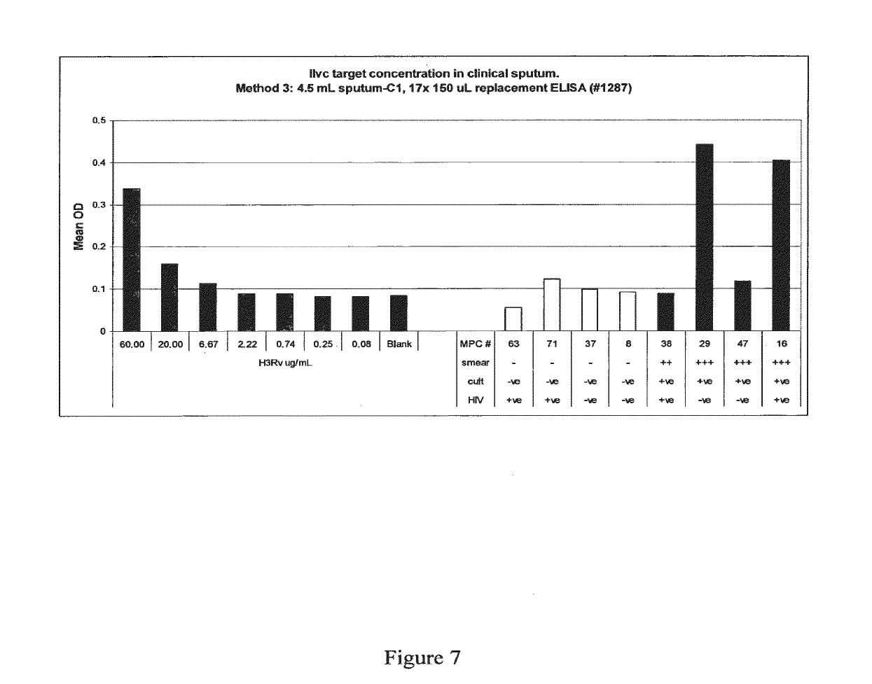

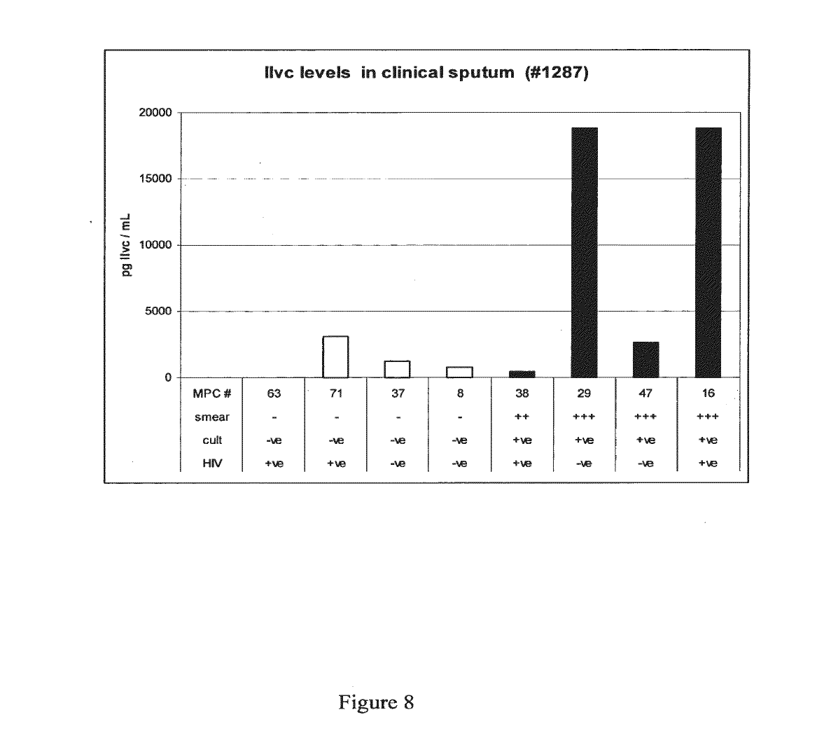

Data presented in FIGS. 7 and 8 show significantly higher levels of KARI protein detected in at least 2 of the 4 TB-positive samples tested that had been shown previously to be culture-positive and smear-positive. In contrast, background signals were detected in all TB-negative samples.

### 9. Evaluation of Signal Inhibition by Sample

To assess whether or not inhibitory or signal-suppressing factors are present in sputa that might adversely affect assay sensitivity e.g., in ELISA or a point-of-care or field test format, sputa samples were spike with 10 ng/mL recombinant M. tuberculosis KARI protein and the resultant samples serially diluted 1 in 27 over three steps. Samples were incubated overnight and assayed by amplified ELISA as described herein above, or assayed immediately.

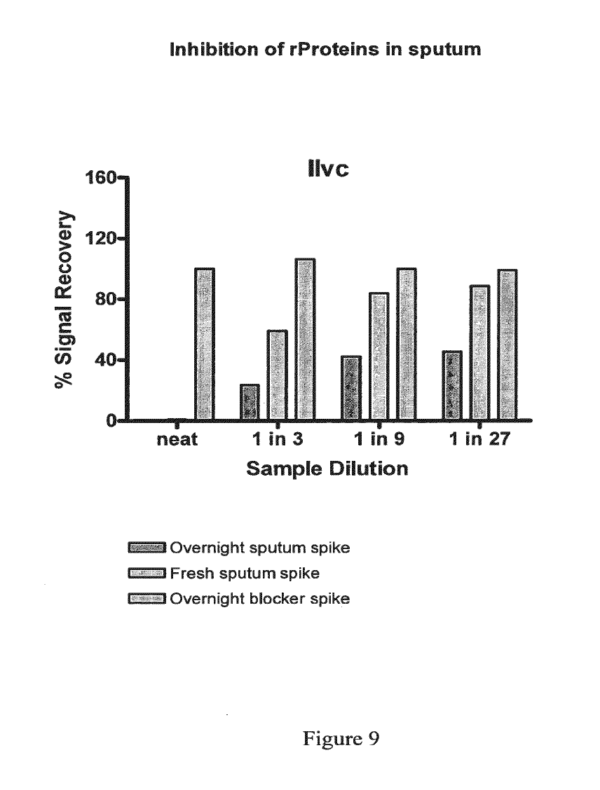

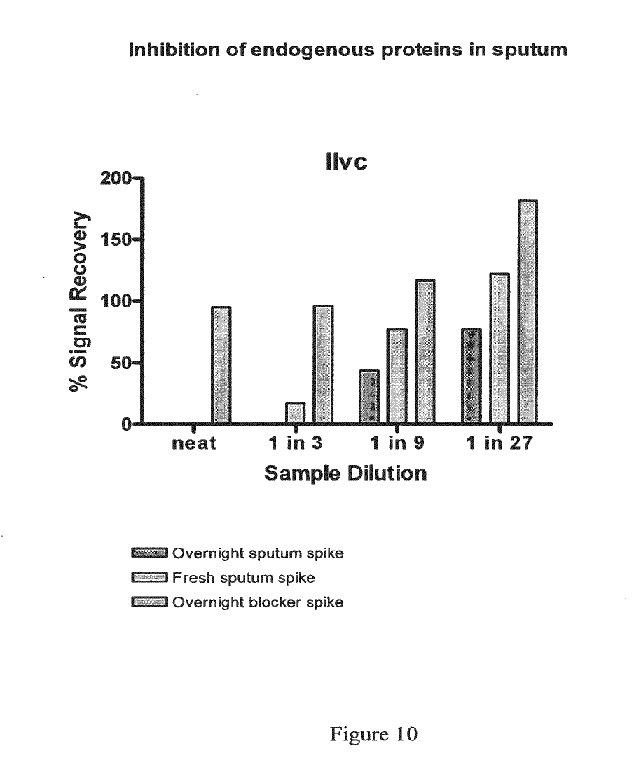

Data presented in FIGS. 9 and 10 (lower right panel marked “ilvC”) indicate that sputum contains some factors that inhibit KARI protein signal detection, since signal strength is reduced following addition of undiluted sputum, irrespective of whether or not the assay is performed immediately or following overnight incubation. However, this loss of signal strength can be progressively inhibited by diluting the sputa, and loss of signal strength is largely prevented by diluting sputum at least about 1 in 9. Signal strength also declines following overnight incubation of recombinant protein in sputum, and that this loss of signal strength can also be partially prevented by dilution of sputum samples. These data indicate that a 1 in 9 dilution of sputum into blocking buffer and rapid assay of samples are recommended to enhance signal strength in assaying KARI protein under these conditions.

### 10. Relative Levels of KARI Protein Detectable in Mycobacteria Cells

To further assess the suitability of KARI as a diagnostic marker for Mycobacteria infection, the levels of KARI protein were determined in whole cell lysates of the M. tuberculosis strains H37Rv, CSU93 and HN878, and in M. tuberculosis, M. avium and M. intracellulaire, relative to 10 other M. tuberculosis antigens including BSX, EF-Tu, P5CR, Rv1265, S9 and TetR-like protein described herein.

Amplified sandwich ELISA was performed essentially as described in this example and in Example 1, to identify relative levels of each antigen according to the standard protocol, with calibration standards included to permit quantitation.

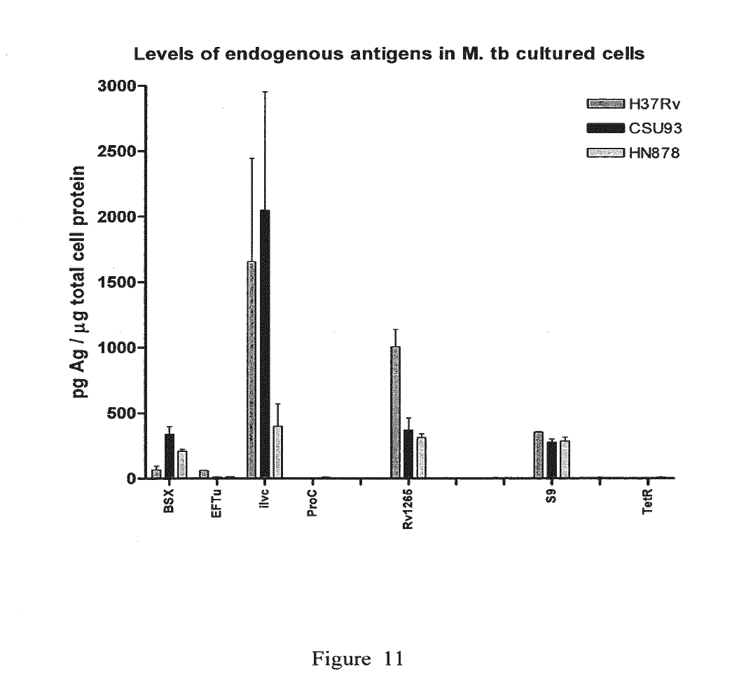

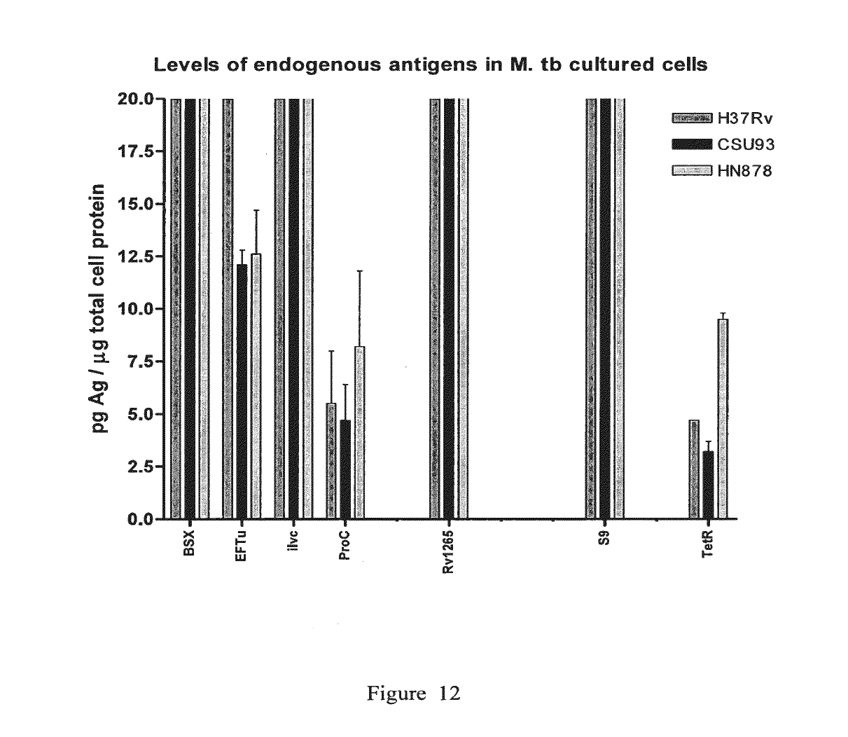

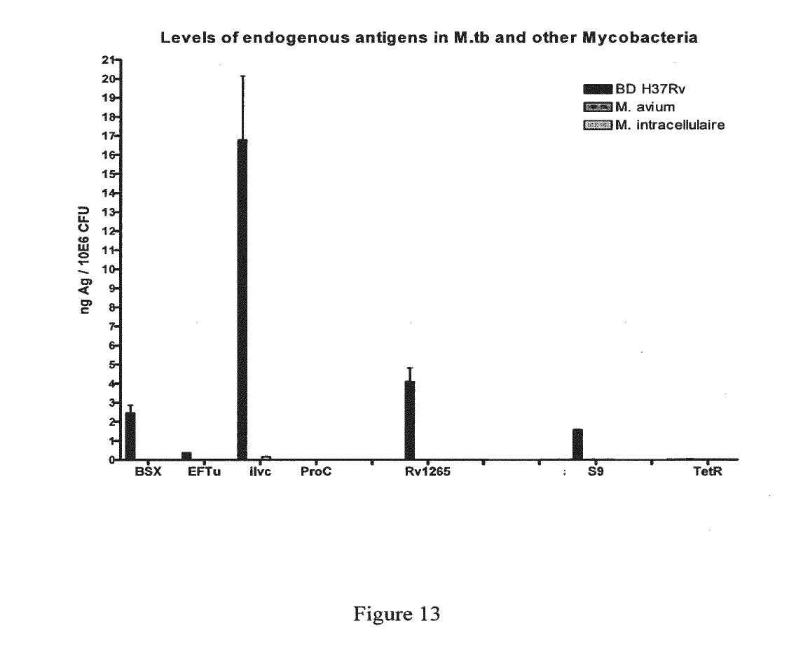

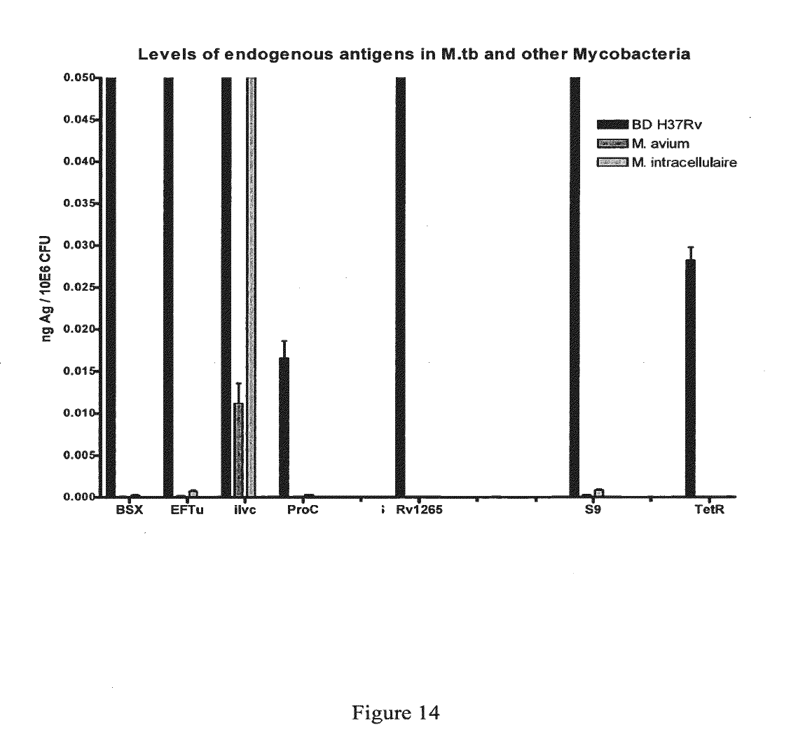

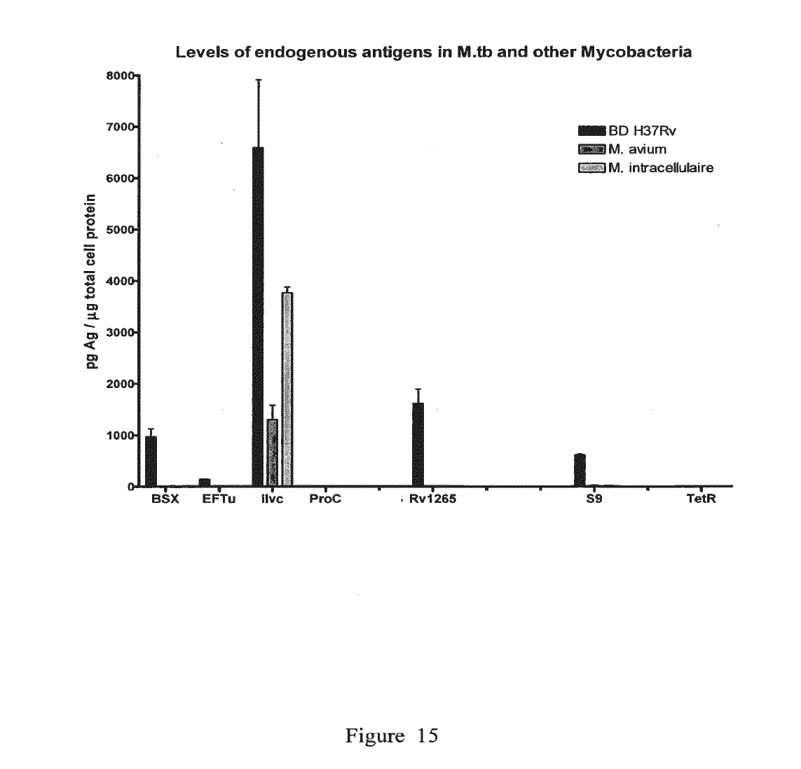

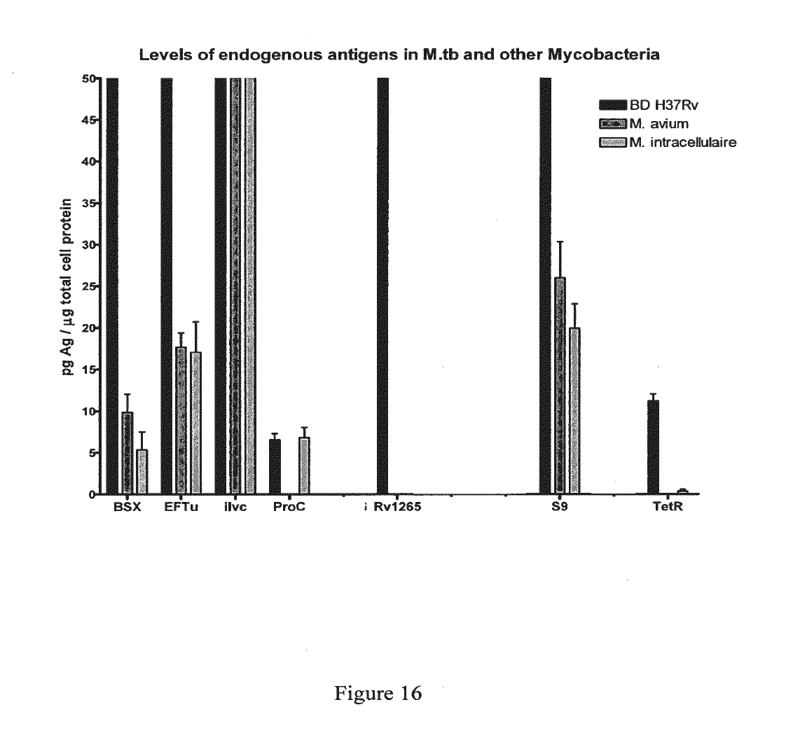

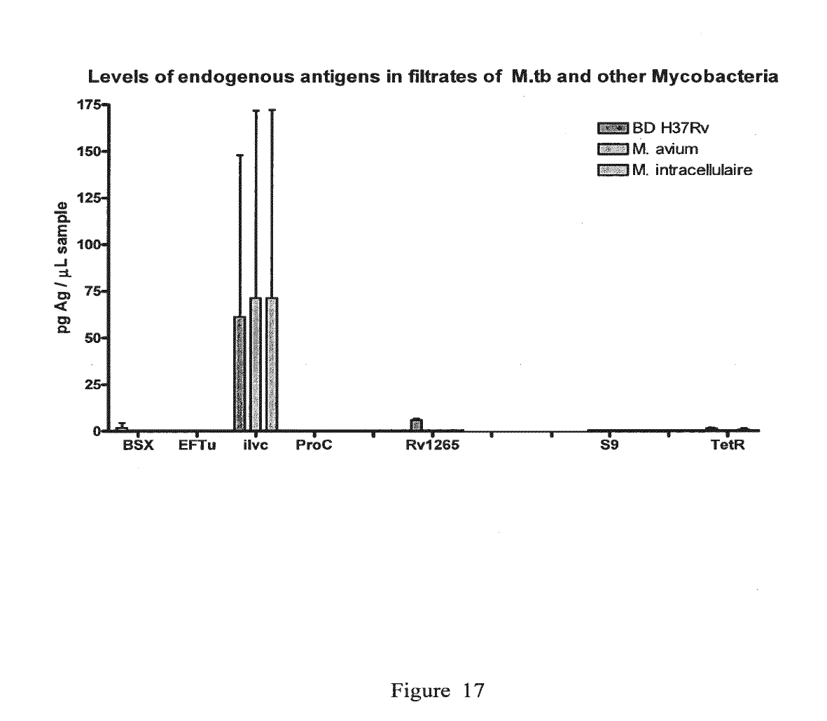

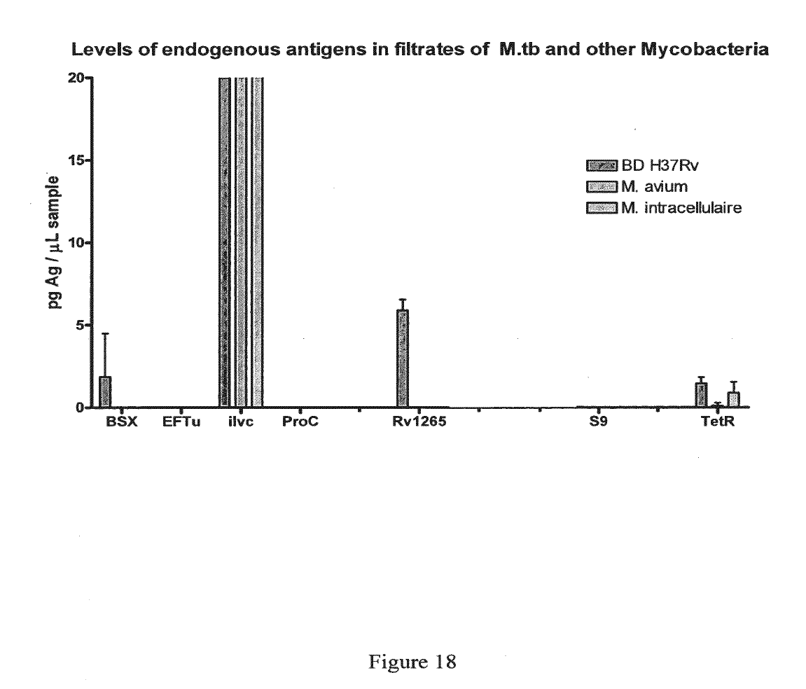

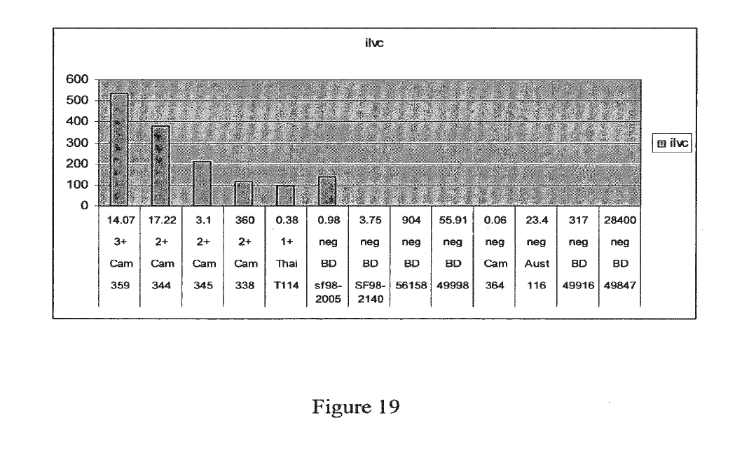

Data shown in FIGS. 11-12 indicate that KARI is a relatively abundant protein in all three M. tuberculosis strains tested when expressed on the basis of total cellular protein. On this basis, M. tuberculosis Rv1265, BSX and S9 proteins are also relatively abundant among the 11 immunogenic proteins tested. Data shown in FIGS. 13-18 indicate that KARI protein is also a relatively abundant protein in Mycobacteria species generally, whereas other predominant immunogenic proteins tested i.e., BSX, Rv1265 and S9, appear to have greater specificity for M. tuberculosis compared to KARI when expressed on a per cell basis (FIGS. 13-14) or as per microgram of whole cell lysate protein (FIGS. 15-16) or as per microlitre of whole cell lysate filtrate (FIGS. 17-18). These data suggest utility of KARI as a generic single-analyte marker of mycobacteria infection, or as part of a multi-analyte test for mycobacteria infection or M. tuberculosis infection in combination with BSX and/or Rv1265 and/or S9 proteins. Other combinations are not excluded for multi-analyte testing of M. tuberculosis infection.

### 11. Optimizing the Limits of Detection

To further enhance sandwich ELISA sensitivity, a replacement amplification procedure is employed to employ iterative antigen binding following coating of the ELISA plate with capture antibody. Essentially, this will result in an increased amount of antigen being bound to the capture antibody notwithstanding the 50 μl volume limitations of a 96-well ELISA plate. Briefly, this iterative antigen loading involves repeating the antigen binding step in the sandwich ELISA several times, e.g., 2 or 3 or 4 or 5 times, etc. before washing and adding detection antibody. Naturally, each aliquot of antigen sample is removed following a standard incubation period before the next aliquot is added. The number of iterations can be modified to optimize the assay (e.g., parameters such as signal:noise ratio, detection limit and amount of antigen detected at half-maximum signal), depending upon the nature of the sample being tested (e.g., sample type), without undue experimentation. For example, up to about 20 iterations of sample loading (i.e., up to a 20× replacement amplification) can be employed to provide a low background signal, and a reduced detection limit of M. tuberculosis KARI protein.

### Example 4

### Relative Specificity of ilvC Assay Towards the M.tb. Complex

The monoclonal antibody Mo2B1 was prepared employing a cell culture supernatant of the 2B1 cell line recognizing KARI protein and paired with Chicken antibody 34/35 as detector, as described in Examples 1 and 3. Optimization of ELISA using this antibody pair was done as described in Example 3.

### 1. Limits of Detection

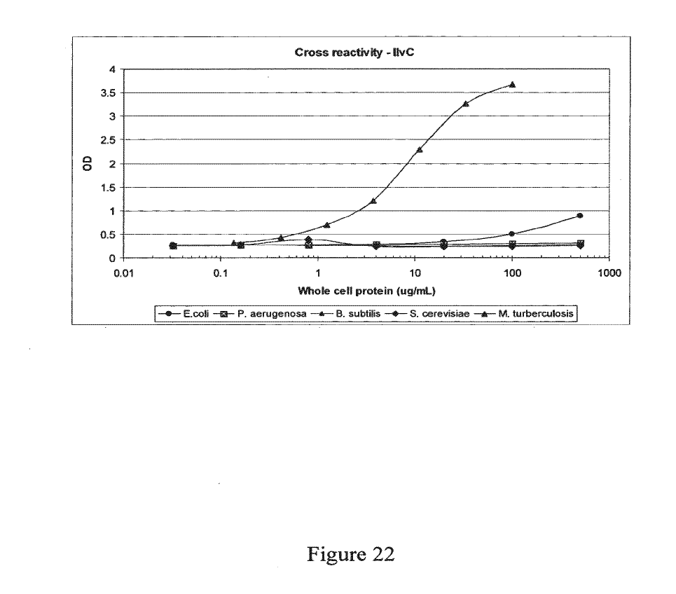

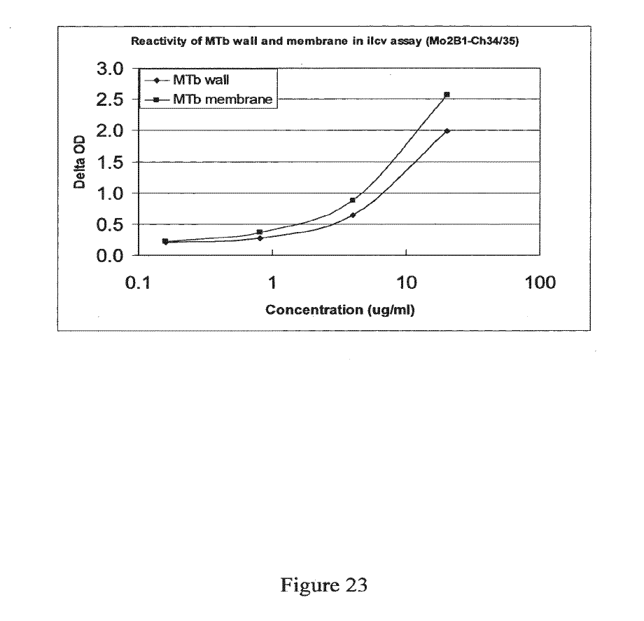

ELISA assays for four markers were assessed for limit of detection and working range determined using recombinant protein and H37Rv whole cell lysate. Standard curves were prepared in buffer (FIG. 22) and in sputum. The working ranges are tabulated in Table 4.

| Range of ELISA | | | | | |
|---|---|---|---|---|---|
| detection (pg/mL) | IlvC | BSX | Rv1265 | S9 | |
| LOD | 200 | 12 | 32 | 38 | 25 |
| EC50 | 2039 | 96 | >5000 | >1500 | |
| Upper limit | 13679 | 517 | >5000 | >1500 | |

All target assays detect M.tb. with a limit of detection below 1 □g H37Rv WCL/mL. The relationship of recombinant target, and expressed protein is clearly shown by FIG. 2.

### 2. Specificity of KARI Antibodies

Irradiated cell suspensions containing known cell numbers were obtained for M. tb. (cell line H37Rv, TMC 102, 1.9×10e9 cfu/mL LOT WA0426A), M. avium, (cell line TMC 724, 1×10 e11 cfu/mL LOT WA0426B), and M. intracellulaire (cell line TMC 6450, 25×10e10 cfu/mL LOT WA0426C). These cell suspensions were applied directly to the ELISA as serial dilutions in buffer to determine the level of target present in whole cell suspension. A 300 uL aliquot of each cell type was also taken and used to prepare whole cell lysate as described above (Preparation of M.tb and recombinant antigen standards). The three cell lines were processed under identical conditions and dilution to ensure fair comparison of the lysates.

The standard curve data for M.tb., M. avium, and M. intracellulaire was assessed against the response given by purified recombinant protein for each target, and this data used to provide information on the level of expression of each target per microgram of whole cell lysate protein (note this figure includes a small contribution from BSA used as a blocker in the bead mill process), and per 10e6 cells in culture (Table 2A-D).

The reactivity of all four assays was further characterized for the common bacterial targets Escherichia coli, Pseudomonas aeruginosa, Bacillus subtilis, and to the yeast, Saccharomyces cerevisiae. Data are shown for ilvc (FIG. 3), however the other three assays showed similar profiles, with no significant cross-reactivity to these non-target organisms. These results established the relative specificity of the newly developed ilvc assay towards M.tb.

Irradiated whole cell suspensions, and whole cell lysates of cultured mycobacteria were prepared as above. The level of detection of target per million cells was determined as well as level of cross reactivity (CR) relative to detection of M.tb.

### 3. Availability of KARI Antigen in Sub-Cellular Fractions

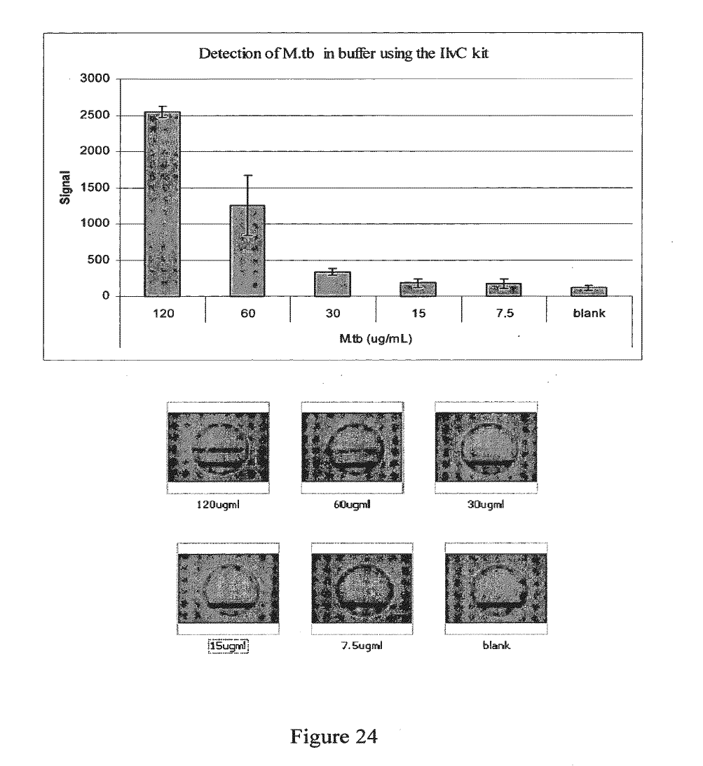

For M.tb., sub-cellular fractions of cell wall and cell membrane were also obtained from the culture of the cell line H37Rv and used to determine the level of available antigen in these preparations. FIG. 24 shows that KARI is present at significant levels in both the M.tb. cell wall and cell membrane preparations.

TABLE 5A-D
Detection of target in cultured cells of M.tb., M. avium, and
M. intracellulaire for the four target assays.

| | pg target per | pg target | Pg/10e6 | % CR |
|---|---|---|---|---|
| | 10e6 whole | per ug | lysed | (per cell basis |
| Cellular target | cells | WCL | cells (WCL) | in WCL) |
| A | Ilvc (Mo2B1 capture assay) | | | |
| M. tb. | 221.3 | 420.9 | 3692.0 | 100 |
| M. avium | 0.649 | 5.5 | 0.92 | .025 |
| M. intracellulaire | 0.014 | 16.6 | 11.1 | 0.3 |

Assay LOD: 200 pg rIlvc/mL
Assay EC₅₀: 2039 pg rIlvc/mL

| B | Rv1265 | | | |
|---|---|---|---|---|
| M. tb. | >1421 | 324 | 2842 | 100 |
| M. avium | 0.136 | 0.06 | 0.029 | 0.0010 |
| M. intracellulaire | 0.192 | 0.06 | 0.037 | 0.0013 |

Assay LOD: 32 pg rRv1265/mL
Assay EC₅₀: >5000 pg rRv1265/mL

| C | BSX | | | |
|---|---|---|---|---|
| M. tb. | 24 | 10.7 | 94 | 100 |
| M. avium | <0.0003 | 0.025 | 0.004 | 0.004 |
| M. intracellulaire | <0.0011 | <0.003 | <0.002 | <0.002 |

Assay LOD: 12 pg rBSX/mL
Assay EC₅₀: 96 pg BSX/mL

| D | S9 | | | |
|---|---|---|---|---|
| M. tb. | >426 | >81 | >710 | 100 |
| M. avium | 0.010 | 0.17 | 0.028 | <0.004 |
| M. intracellulaire | <0.002 | 0.90 | 0.6 | <0.084 |

Assay LOD: 38 pg rS9/mL
Assay EC₅₀: >1500 pg rS9/mL

### Example 5

### Development of a Point-of-Care Assay for the Detection of KARI (ilvC)

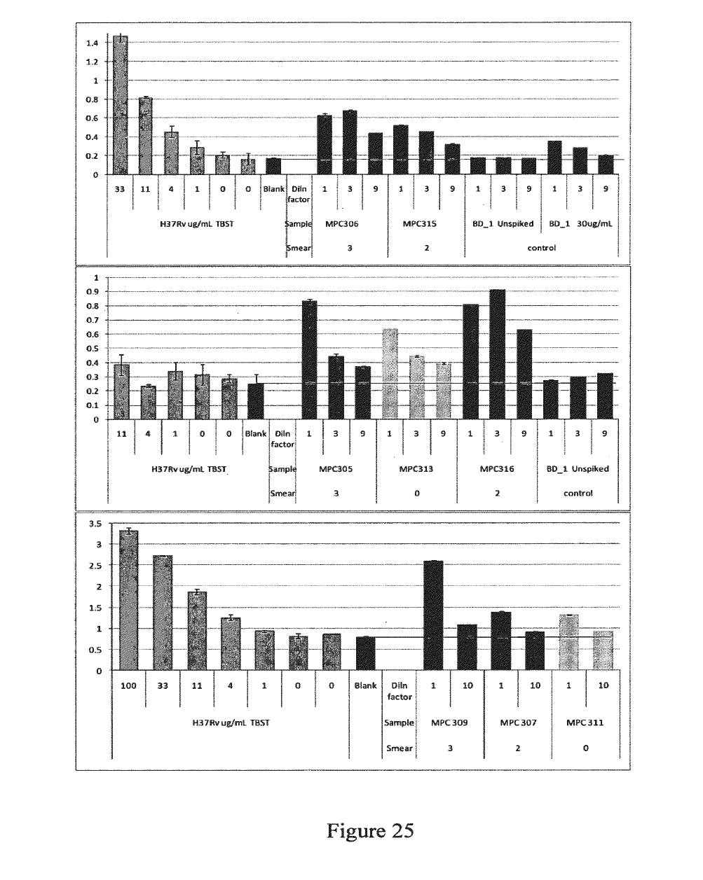

The purified KARI monoclonal antibody 2B1 reagent has been conjugated to gold, and assessed for suitability in the DiagnostIQ assay format, which is a point-of-care test. The standard curve in buffer developed in an un-optimised system using this conjugate with membrane bound Ch34/35 preliminary Data are shown in FIG. 25.

### Example 6

### Screening of Clinical Samples Using Optimized ELISA for KARI

Tyrian Diagnostics has sourced samples from a number of different sites for use in this study e.g., Table 6. Cameroon was the primary study site for this clinical study due to the timely provision of samples with associated clinical data and sufficient volumes for performance of replicate assays. These samples were collected from field clinics where samples could be rapidly processed and “snap frozen”. Patients were screened for HIV status, and where possible CD4 counts obtained to determine immune function. Patients were questioned with respect to prior exposure to TB, and patients on treatment for mycobacterial infection were excluded from the co-hort. Sputum volumes were generally 1-2 mL, following removal of an aliquot for smear and culture determination, with a small number of patients providing volumes of up to 7 mL.

In addition, we tested TB positive samples from Thailand and South Africa. To date, sample received have been small in volume (<1 mL) and predominantly TB negative.

Clinical samples were also screened for ilvc and, where possible, other targets from the four candidate markers. Data in Table 7 are for 26 smear positive patients. Data in Table 8 are for 29 smear negative patients. Samples are recorded as positive if the average signal is greater than 3 standard deviations above the assay buffer blank. Weakly positive signals for ilvc are shown in italics.

TABLE 6
Clinical data for sputum samples analysed in this clinical study.

| | | | | Confirmed | HIV | CD 4 | | |
|---|---|---|---|---|---|---|---|---|
| Source | Sample ID | Smear | Culture | organism? | status | count | Origin | Race |
| BD | 50118 | 0 | 0 | ? | −ve | n/a | BD_USA | |
| BD | 55152 | 0 | 0 | ? | −ve | n/a | BD_USA | |
| CG | 116 | 0 | 0 | ? | Unknown | | QLD_Aus | |
| BD | 1110 | 0 | 0 | ? | −ve | n/a | BD_USA | |
| BD | 50814 | 0 | 0 | ? | −ve | n/a | BD_USA | |
| BD | 50231 | 0 | 0 | ? | −ve | n/a | BD_USA | |
| BD | 56177 | 0 | 0 | ? | −ve | n/a | BD_USA | |
| BD | 50144 | 0 | 0 | ? | −ve | n/a | BD_USA | |
| CG | 115 | 0 | Pending | ? | Unknown | | QLD_Aus | |
| BD | 50359 | 0 | 0 | ? | −ve | n/a | BD_USA | |
| Thai | 112 | 0 | 0 | ? | −ve | n/a | Thailand | |
| BD | 1229 | 0 | 0 | ? | −ve | n/a | BD_USA | |
| BD | 1288 | 0 | 0 | ? | −ve | n/a | BD_USA | |
| MPC | 364 | 0 | 0 | ? | +ve | 169 | Cameroon | Black |
| BD | 1287 | 0 | 0 | ? | −ve | n/a | BD_USA | |
| BD | 1187 | 0 | 0 | ? | −ve | n/a | BD_USA | |
| MPC | 363 | 0 | 0 | ? | +ve | ? | Cameroon | Black |
| CG | 123 | 0 | Pending | ? | Unknown | | QLD_Aus | |
| MPC | 375 | 0 | 0 | 0 | +ve | ? | Cameroon | |
| PHRU | 905 | 0 | 0 | 0 | −ve | n/a | South Africa | |
| CG | 119 | 0 | Pending | ? | Unknown | | QLD_Aus | |
| MPC | 388 | 0 | 0 | 0 | −ve | n/a | Cameroon | |
| BD | 505 | 0 | 0 | ? | −ve | n/a | BD_USA | |
| MPC | 339 | 0 | 0 | M. tuberculosis | +ve | 299 | Cameroon | Black |
| MPC | 311 | 0 | Pending | ? | −ve | ? | Cameroon | |
| MPC | 313 | 0 | 0 | ? | +ve | ? | Cameroon | Black |
| BD | BD Yellow | 0 | 0 | ? | −ve | n/a | BD_USA | |
| BD | BD2_67845 | 0 | 0 | ? | −ve | n/a | BD_USA | |
| BD | BD2_white | 0 | 0 | ? | −ve | n/a | BD_USA | |
| | 10014301 & | | | | | | | |
| | 14298 | | | | | | | |
| MPC | 379 | 1 | 0 | ? | −ve | n/a | Cameroon | |
| MPC | 372 | 1 | Moderate | M. tuberculosis | −ve | n/a | Cameroon | Black |
| MPC | 360 | 2 | High | M. tuberculosis | +ve | ? | Cameroon | Black |
| MPC | 356 | 3 | High | M. tuberculosis | +ve | 181 | Cameroon | Black |
| MPC | 378 | 2 | 0 | ? | −ve | n/a | Cameroon | |
| MPC | 365 | 2 | 0 | ? | +ve | ? | Cameroon | |
| MPC | 335 | 2 | High | M. tuberculosis | +ve | 22 | Cameroon | Black |
| MPC | 324 | 2 | High | M. tuberculosis | +ve | 170 | Cameroon | Black |
| MPC | 368 | 2 | High | M. tuberculosis | +ve | 210 | Cameroon | Black |
| MPC | 307 | 2 | Mod | M. africanum | −ve | n/a | Cameroon | Black |
| MPC | 315 | 2 | High | M. tuberculosis | +ve | 587 | Cameroon | Black |
| MPC | 316 | 2 | | ? | +ve | ? | Cameroon | |
| MPC | 374 | 3 | 0 | ? | −ve | n/a | Cameroon | |
| MPC | 366 | 3 | High | M. tuberculosis | −ve | n/a | Cameroon | Black |
| MPC | 357 | 3 | High | M. tuberculosis | −ve | n/a | Cameroon | Black |
| MPC | 361 | 3 | High | M. tuberculosis | −ve | n/a | Cameroon | Black |
| MPC | 381 | 3 | 0 | ? | −ve | n/a | Cameroon | |
| MPC | 380 | 3 | 0 | ? | −ve | n/a | Cameroon | |
| MPC | 370 | 3 | High | M. tuberculosis | −ve | n/a | Cameroon | Black |
| MPC | 342 | 3 | High | M. tuberculosis | −ve | n/a | Cameroon | Black |
| MPC | 377 | 3 | 0 | ? | +ve | 0 | Cameroon | |
| MPC | 367 | 3 | High | M. tuberculosis | +ve | ? | Cameroon | Black |
| MPC | 359 | 3 | High | M. tuberculosis | +ve | 213 | Cameroon | Black |
| MPC | 305 | 3 | | | +ve | | Cameroon | |
| MPC | 306 | 3 | | | +ve | | Cameroon | |
| MPC | 309 | 3 | | | −ve | n/a | Cameroon | |

TABLE 7
Detection of smear positive clinical samples.

| | Sam- | | | | | |
|---|---|---|---|---|---|---|
| | ple | | | | | |
| smear | ID | Source | ILVC | Rv1265 | BSX | S9 |
| 1 | 379 | Cameroon | POSITIVE | | | negative |
| 1 | 372 | Cameroon | POSITIVE | | negative | questionable |
| 2 | 360 | Cameroon | POSITIVE | negative | negative | negative |
| 3 | 356 | Cameroon | POSITIVE | POSITIVE | negative | negative |
| 2 | 378 | Cameroon | POSITIVE | | | negative |
| 2 | 365 | Cameroon | POSITIVE | | negative | negative |
| 2 | 335 | Cameroon | POSITIVE | | negative | negative |
| 2 | 324 | Cameroon | POSITIVE | | negative | negative |
| 2 | 368 | Cameroon | POSITIVE | | negative | negative |
| 2 | 307 | Cameroon | POSITIVE | POSITIVE | negative | negative |
| 2 | 315 | Cameroon | POSITIVE | POSITIVE | negative | negative |
| 2 | 316 | Cameroon | POSITIVE | POSITIVE | negative | negative |
| 3 | 374 | Cameroon | POSITIVE | POSITIVE | negative | negative |
| 3 | 366 | Cameroon | POSITIVE | POSITIVE | negative | negative |
| 3 | 357 | Cameroon | POSITIVE | negative | negative | POSITIVE |
| 3 | 361 | Cameroon | POSITIVE | negative | negative | negative |
| 3 | 381 | Cameroon | POSITIVE | | | negative |
| 3 | 380 | Cameroon | POSITIVE | | | negative |
| 3 | 370 | Cameroon | POSITIVE | | negative | negative |
| 3 | 342 | Cameroon | POSITIVE | | negative | negative |
| 3 | 377 | Cameroon | POSITIVE | | negative | negative |
| 3 | 367 | Cameroon | POSITIVE | | negative | negative |
| 3 | 359 | Cameroon | POSITIVE | | negative | questionable |
| 3 | 305 | Cameroon | POSITIVE | negative | negative | negative |
| 3 | 306 | Cameroon | POSITIVE | POSITIVE | negative | negative |
| | 309 | Cameroon | POSITIVE | POSITIVE | negative | POSITIVE |
| | | Total | 26 | 12 | 22 | 26 |
| | | samples | | | | |
| | | Total | 0 | 4 | 22 | 22 |
| | | negative | | | | |
| | | Total | 26 | 8 | 0 | 2 |
| | | positive | | | | |

TABLE 8
Detection of smear negative clinical samples.

| | Sample | | | | | |
|---|---|---|---|---|---|---|
| smear | ID | source | ILVC | Rv1265 | BSX | S9 |
| 0 | 50118 | BD-USA | negative | POSITIVE | | negative |
| 0 | 55152 | BD-USA | POSITIVE | POSITIVE | | negative |
| 0 | 116 | QLD-Aus | negative | POSITIVE | | negative |
| 0 | 1110 | BD-USA | POSITIVE | POSITIVE | | negative |
| 0 | 50814 | BD-USA | POSITIVE | POSITIVE | | negative |
| 0 | 50231 | BD-USA | POSITIVE | POSITIVE | | negative |
| 0 | 56177 | BD-USA | negative | negative | | negative |
| 0 | 50144 | BD-USA | negative | POSITIVE | | negative |
| 0 | 115 | Qld-Aus | negative | POSITIVE | | negative |
| 0 | 50359 | BD-USA | POSITIVE | POSITIVE | | negative |
| 0 | 112 | Thailand | POSITIVE | POSITIVE | | questionable |
| 0 | 1229 | BD-USA | negative | POSITIVE | negative | negative |
| 0 | 1288 | BD-USA | negative | negative | negative | negative |
| 0 | 364 | Cameroon | POSITIVE | negative | negative | negative |
| 0 | 1287 | BD-USA | negative | POSITIVE | negative | negative |
| 0 | 1187 | BD-USA | negative | POSITIVE | POSITIVE | negative |
| 0 | 363 | Cameroon | POSITIVE | POSITIVE | POSITIVE | negative |
| 0 | 123 | Qld-Aus | negative | | | questionable |
| 0 | 375 | Cameroon | POSITIVE | | | questionable |
| 0 | 905 | South Africa | negative | | | negative |
| 0 | 119 | Qld-Aus | negative | | | POSITIVE |
| 0 | 388 | Cameroon | POSITIVE | | | negative |
| 0 | 505 | BD-USA | negative | | negative | negative |
| 0 | 339 | Cameroon | POSITIVE | | negative | negative |
| 0 | 311 | Cameroon | POSITIVE | POSITIVE | negative | negative |
| 0 | 313 | Cameroon | POSITIVE | negative | negative | negative |
| 0 | BD Yellow | BD-USA | POSITIVE | POSITIVE | negative | negative |
| 0 | BD2_67845 | BD-USA | negative | POSITIVE | POSITIVE | negative |
| 0 | BD2_white | BD-USA | negative | negative | negative | negative |
| | 10014301 & | | | | | |
| | 14298 | | | | | |
| | | Total samples | 29 | 22 | 13 | 29 |
| | | Total negative | 15 | 5 | 10 | 25 |
| | | Total positive | 14 | 17 | 3 | 1 |

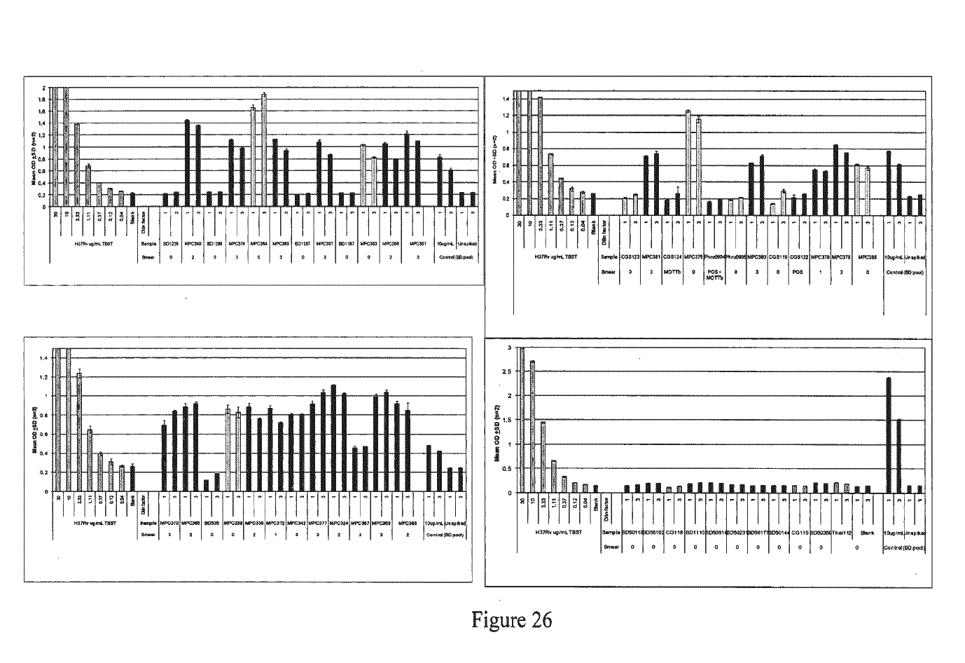

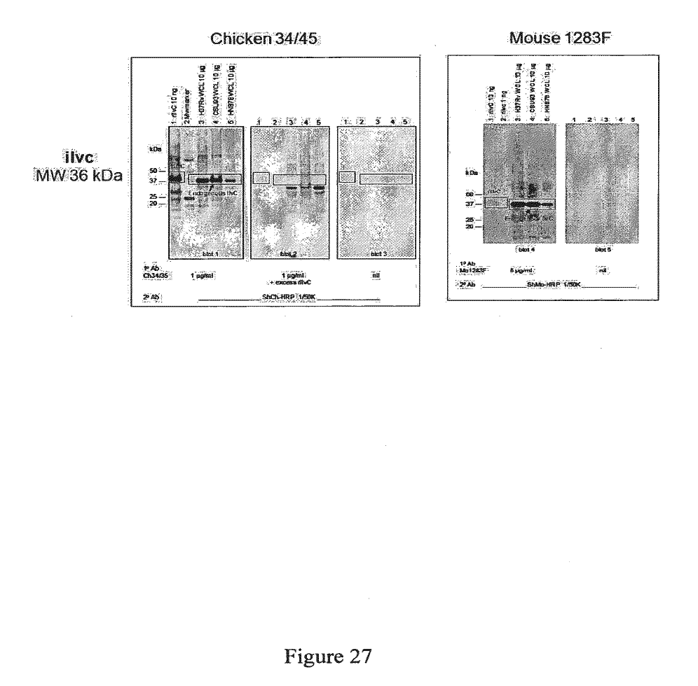

Results shown in FIGS. 26 and 27. For the initial screening of clinical sample, the focus was on applying the maximum amount of sputum to the assay. No attempt (beyond processing at the collection site) was employed to solubilise the sample or remove any interfering components in sputum. The equivalent of 4.5 mL of unprocessed sputum was analysed, and the results are shown in FIG. 6. Two out of 4 TB positive samples showed significant detection of ilvc. In contrast, no significant detection of S9 was observed. Results are shown in FIG. 31.

### Example 7

### Further Characterization of Specific Antibodies to KARI Protein

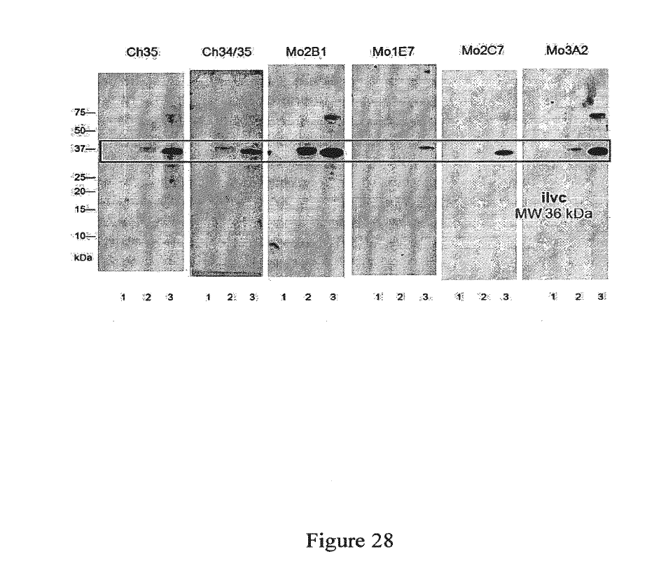

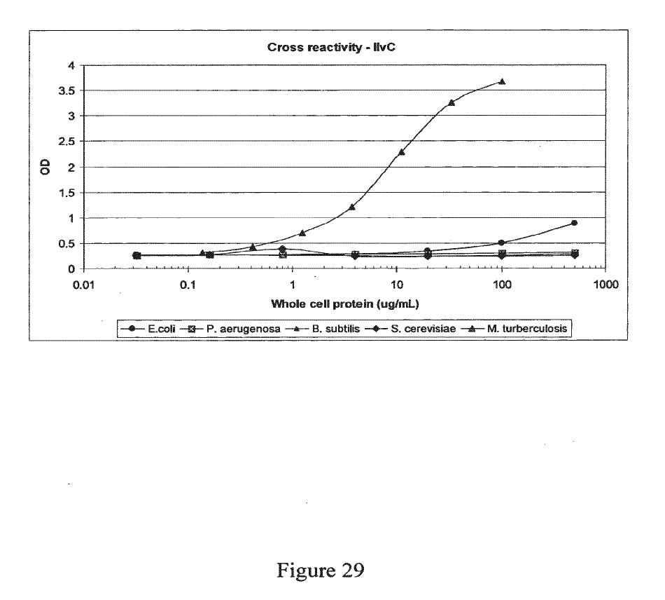

The inventors have found that KARI protein of M.tb. is highly immunogenic, and has multiple epitopes that are capable of producing an immune response. Antibodies have been raised in chicken and in mice immunised with recombinant KARI protein, and/or synthesised peptide antigens. FIGS. 28 and 29 show the antibody specificity of antibodies raised in chicken, and mouse plasmacytoma towards recombinant and endogenous KARI protein.

Irradiated whole cell suspensions, and whole cell lysates of cultured mycobacteria were prepared as above. The level of detection of target per million cells was determined as well as level of cross reactivity (CR) relative to detection of M.tb. (Table 9).

TABLE 9
Level of detection and cross-reactivity for ilvc assay.

| | pg target per | pg target | | % CR (per |
|---|---|---|---|---|
| | 10e6 whole | per ug | Pg/10e6 | cell basis in |
| Cellular target | cells | WCL | lysed cells | WCL) |
| M. tb. | 221.3 | 420.9 | 3692.0 | 100 |
| M. avium | 0.649 | 5.5 | 0.92 | .025 |
| M. intracellulaire | 0.014 | 16.6 | 11.1 | 0.3 |

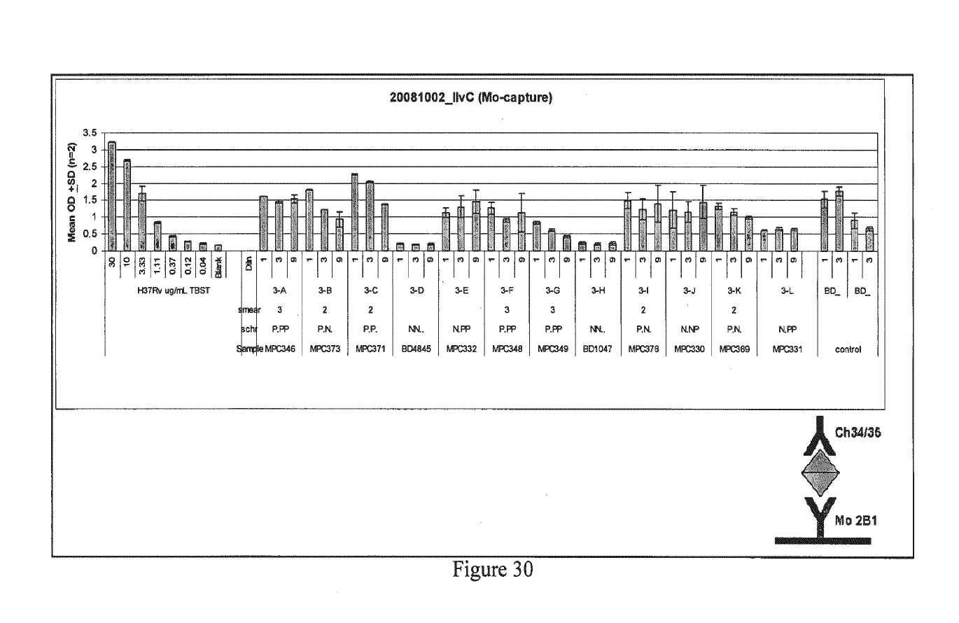

The sensitivity and specificity of ELISA assay in mouse Mo2B1-Chicken 34/35 format was determined and shown in Table 9. FIG. 30 shows the specificity of the ELISA assay. These data show that the assay limit of detection was <200 pg rIlvC/mL, and EC₅₀ was 2039 pg rIlvC/mL. Cross-reactivity to mycobacterial relative to M.tb. was less than 0.3%.

### Example 8

### Design and Testing of Primer Pairs for Amplification of ilvC Transcript from M.tb and/or M.tb Complex by Quantitative PCR (qPCR)

This example demonstrates advantageous primer pairs for use in the quantitative PCR assay described in Example 2 hereof and determination of amplification conditions for achieving specific amplification of ilvC sequences from M.tb and M.tb complex.

1. DNA Preparation from Cultured M. tuberculosis

Cell lysates were prepared incubating M. tuberculosis cells in breaking buffer (50 mM Tris-HCl pH 8.0; 10 mM EDTA; 100 mM NaCl; 0.6% SDS; proteinase K) for 12 h at 50° C. and disrupting cells by beadbeating. The cell lysates were centrifuged to remove debris before being frozen. Frozen lysates were maintained on dry ice during short-term storage before being processed further. DNA was extracted by adding an equal volume of Tris-buffered (pH 8) phenol:chloroform:isoamyl alcohol (25:24:1; Fluka) to each lysate and vortexing mixtures for 20 sec, centrifuging samples at 13,000 rpm for 5 min in a bench-top centrifuge to separate layers. The upper layer (containing DNA) was carefully removed and transferred to a clean Eppendorf tube and an equal volume of cold isopropanol added. The mixture was briefly vortexed and left to precipitate overnight at −20° C. Following precipitation, the sample was spun at 13,000 rpm for 20 min at 4° C. to pellet DNA. Supernatant was then discarded and the pellet washed with 70% ethanol and spun at 13,000 rpm for another 20 min at 4° C. Following final precipitation, supernatant was discarded and the pellet allowed to air-dry for 10 min before being eluted in water. Final DNA concentration was determined by nano-drop spectrophotometry.

2. Quantitative Real-Time PCR (qPCR) Assay

qPCR assays were performed on an ABIPrism-7500 Sequence Detector System over 40 (or otherwise stated) cycles of 95° C. for 15 sec and 60° C. for 1 min in a two-step thermal cycle, preceded by an initial 10 min step at 95° C. Genomic DNA was originally diluted to 100 ng/μl and 1 μl used in a 25 μl qPCR reaction (i.e. 4 ng total), containing SYBR green PCR master mix (Applied Biosystems) and 150 nM each forward and reverse primers. Amplicons were initially analysed by 2% agarose gel electrophoresis, and dissociation curve analyses were performed for each experiment to monitor the amplification of single products. Standard curve analyses were performed on 6 serial ten-fold dilutions in triplicate. The ABIPrism-7500 SDS Software was used for threshold (Ct) data acquisition, dissociation curve and standard curve analyses. Primers, which included SEQ ID NOs: 19 and 20 (Example 2), are listed in Table 10.

### 3. In Silico PCR Analysis

In silico PCR simulation was performed on-line against Mycobacterium species and other stipulated exclusion organisms: Pseudomonas, Haemophilus, Streptococcus and Staphylococcus. The primer pairs were not screened against Moraxella. No amplification was detected by primer pairs ilvC1, ilvC2 or ilvC3 against exclusion organisms and only subspecies M. tuberculosis and M. bovis showed predicted amplification when screened against all Mycobacterium spp.

### 4. Amplification Specificities and Efficiencies of the Primer Pairs

Primer-specific amplification was obtained using 4 ng DNA extracted from M. tuberculosis, per reaction. Amplicons of the predicted sizes were obtained only in the presence of template nucletic acids. Primer pairs ilvC1, ilvC2 and ilvC3 each produced single products as determined by dissociation analysis. Primer pair rtM.tbilvCF/rtM.tbilvCR produced a secondary product after extended PCR cycling (+30 cycles). Empirically-determined melting temperatures of the amplicons are shown in Table 10. Standard curve analysis was performed on DNA extracted from M. tuberculosis spiked with trace amounts of primer-specific amplicons and performed on 6 serial ten-fold dilutions in triplicate. Primer-specific amplification efficiency (E) is defined by the formula E=10^{(-1/slope)}, and converted to percentage values by the formula (E-1)×100% as described by Rasmussen (2001), “Quantification on the LightCycler”, In: Rapid cycle real-time PCR, methods and applications (Springer Press, Heidelberg, S. Meuer, C. Wittwer and K. Nakagawara eds). Amplification efficiencies are presented in Table 10.

5. Primer Performance at Different DNA Concentration and/or Cycle Number

Amplification of unspecific and/or low primer-sequence homology targets is normally observed with either high input DNA concentration and/or high PCR-cycling number. Accordingly, the primer pairs shown in Table 10 were tested in a qPCR reaction at 40 cycles with 400 pg input DNA per reaction (i.e. relatively high DNA concentration) at variable cycle number. There was a shift in Ct-values at around 12 cycles for these primers when assayed at same input DNA concentration. At 400 pg input DNA concentration, amplification of ilvC sequences from M. Tb+ samples was observed to cross a threshold baseline level at about 17 cycles for SEQ ID NOs: 27-32 and at about cycle 22 for SEQ ID NOs: 19 and 20. Amplification products at this high DNA concentration were detectable for primer pairs tested in negative samples, albeit only crossing threshold baseline limits of detection at about cycle 29 for SEQ ID NOs: 27-32 and at about cycle 36 for SEQ ID NOs: 19 and 20.

With a shift in Ct-values at around 12 cycles for these primers when assayed at same input DNA concentration, detection of specific M.tb+ilvC sequences, i.e., excluding non-specific amplification, is expected at lower input DNA concentrations and/or lower cycle number.

A further qPCR assay was performed using the primer pairs set forth in Table 10 albeit at a 10-fold lower input DNA concentration than previously i.e., about 40 pg DNA, and for 30 cycles of amplification. With amplification efficiencies at around 90% (Table 10), this 10-fold lower DNA concentration corresponds to a downwards shift of 3.5 cycles, i.e. amplification crossing base line approximately 3.5 cycles after that observed for the higher DNA concentration. Under these conditions, all primer pairs shown in Table 10 produced amplification products for M.tb+ positive samples, and there was no detectable amplification product for M.tb negative samples after 30 cycles. As expected, amplification crossed base line approximately 3.5 cycles after that observed at higher DNA concentration e.g., at about 20-21 cycles for SEQ ID NOs: 27-32 and at about 25-26 cycles for SEQ ID NOs: 19 and 20. Dissociation curves (not shown) also showed amplification of single products for SEQ ID NOs: 27-32 and some unspecific product using SEQ ID NOs: 19 and 20.

6. Summary of Primer Performance in qPCR

In this study, the four primer pairs shown in Table 10 were tests for their specificity and efficiency in amplifying M. tuberculosis ilvC sequences and/or M. tuberculosis complex ilvC sequences from biological specimens e.g., sputum. For an assay to detect M. tuberculosis complex ilvC sequences specifically, excluding other sources such as M. avium, care must be made towards ensuring appropriate input DNA concentration and PCR-cycle number to thereby reduce specificity of the amplification. The data herein suggest that PCR amplification will occur where the input template concentration is high and/or PCR-cycling is extended beyond about 25-30 cycles. For example, a high concentration of M. avium template may result in amplification of those sequences when cycle number exceeds about 25-30 cycles. However, for specific detection of M. tuberculosis complex i.e., including both M. tuberculosis and M. bovis, it is preferred to employ the optimal DNA concentration and cycling parameters herein.

TABLE 10
Primer sequences for amplifying IlvC nucleic acid and size of PCR amplicons

| | | | Amplification | | SEQ |
|---|---|---|---|---|---|
| | Amplicon | Amplicon | efficiency | | ID |
| Primer | size (bp) | Tm | (%) | Primer Sequence | NO: |
| rtM.tbilvCF | 83 | 82-83° C. | 76.7 | GGCAACAAACAGCTCGAAGAG | 19 |
| rtM.tbilvCR | | | | CATCAGGTCGCGGAGTTTCT | 20 |
| ilvC1F | 130 | 88° C. | 89.8 | AGGTTGGTGTGATCGGCTA | 27 |
| ilvC1R | | | | AGGCCCTGCTCTTCTACCTT | 28 |
| ilvC2F | 80 | 84° C. | 91.7 | GTGTGCCGTGTTTGGTTG | 29 |
| ilvC2R | | | | GATCGCTTTGGCATACGAC | 30 |
| ilvC3F | 109 | 85° C. | 85.0 | TGGCGCTGTCGTATGCCAA | 31 |
| ilvC3R | | | | CACAACACCGTTTGCTCACC | 32 |

The data herein also demonstrate that SEQ ID NOs: 27-32 are preferred for qPCR to detect M. tuberculosis and/or M. tb complex, however all four primer pairs are useful in this context. An order of preference of the primer pairs, from most preferred to less preferred, is as follows:
SEQ ID NOs: 29-30≧SEQ ID NOs: 27-28>SEQ ID NOs: 31-32>SEQ ID NOs: 19-20.

It is preferred not to interchange the forward and reverse primers described herein.

Based on their alignments in the ilvC coding sequence (SEQ ID NO: 2), it is possible to cross-combine primer pairs, especially SEQ ID NOs: 27-32, or more preferably SEQ ID NOs: 29-32, to thereby create additional primer pairs for use in the amplification protocol, subject to the exclusion of a cross-combination of overlapping primers having SEQ ID NOs: 30 and 31, which is not desirable. For example, the additional primer combinations set forth in Table 11 may be employed:

TABLE 11
Useful primer combinations

| | Forward primer | | | | |
|---|---|---|---|---|---|
| | | ilvC1F | ilvC2F | ilvC3F | rtM.tbilvCF |
| Reverse primer | ilvC1R | Yes | No | No | No |
| | ilvC2R | Yes | Yes | No | No |
| | ilvC3R | Yes | Yes | Yes | No |
| | rtM.tbilvCR | Yes | Yes | Yes | Yes |

### Example 9

### Detection of M. tuberculosis Complex Organisms by Quantitative RT-PCR in Clinical Samples

This example demonstrates the utility of quantitative RT-PCR for detecting M.tb complex organisms in culture samples, and clinical samples e.g., sputa, fine needle aspirates of lymph node tissue, broncho alveolar lavage (BAL).

### 1. Samples

Culture samples used in this example were suspensions of two clinical strains of M. tuberculosis designated A and B Starting at McFarland 0.5 (150×10⁶ CFU/mL), serial dilutions were made equivalent to 10⁸ CFU/mL, 10⁶ CFU/mL, 10⁴ CFU/mL, 10² CFU/mL, 10 CFU/mL and 1 CFU/mL. Two additional culture extracts were supplied by Tyrian Diagnostics.

Control sputa used in this example, designated A-G, were supplied by the Royal College of Pathologists Australasia (RCPA) QAP programme. Stock sputa contained 60-85 AFB/100HPF by microscopy. Samples A and D were negative for M.tb. Samples A, C and E were also spiked with dilute concentrations of M. avium.

Additionally, four archived complete clinical samples previously tested at ICPMR were tested. These clinical samples consisted of two sputa, one Fine Needle Aspirate of Lymph Node tissue, and one Broncho Alveolar Lavage.

### 2. RNA Extraction

RNA was extracted from each culture sample and each clinical sample using the NucliSENS® easyMAG® platform (bioMérieux, North Carolina, USA) according to the manufactuer's protocol. EasyMag is an ND-labeled automated system for the extraction of total nucleic acids from a variety of sample types and volumes, optimized for extraction of total nucleic acid from biological samples. The system automates an enhanced magnetic silica version of bioMérieux's BOOM® technology. Extracted nucleic acid was eluted in 110 uL aliquots and treated with DNAse, thereby yielding RNA.

### 3. Reverse Transcription

cDNA was produced from each RNA samples by reverse transcription using the SuperScript cDNA synthesis kit (Invitrogen Corporation, USA) according to the manufactuer's protocol.

### 4. Quantitative PCR

Each cDNA sample was used as a template for separate PCR reactions containing primer sets provided in Table 10 hereof.

PCR was performed essentially as in the preceding example employing a Roche LC 480 platform, albeit with a first incubation at 50° C. for 2 min followed by 95° C. for 5 mins, and then 30 to 50 cycles each at 95° C. for 10 seconds followed by 60° C. for 45 seconds. Reactoins were completed by a final incubation at 40° C. for 5 min.

### 5. Results

As indicated in the preceding example, less non-specific amplification product was produced when the cycle number was reduced from 50 cycles to about 30 cycles. Products having the expected Tm values from melt curves were amplified using each of the primer pairs shown in Table 10 hereof.

In culture samples, a primer pair consisting of SEQ ID NOs: 27 and 28 detected specific M.tb complex ilvC at concentrations of at least about 10⁴ CFU/mL, and a primer pair consisting of SEQ ID NOs: 29 and 30 detected specific M.tb complex ilvC at concentrations of at least about 10⁴ CFU/mL, and a primer pair consisting of SEQ ID NOs: 31 and 32 detected specific M.tb complex ilvC at concentrations of at least about 10⁴ CFU/mL, and a primer pair consisting of SEQ ID NOs: 19 and 20 detected specific M.tb complex ilvC at concentrations of at least about 10⁶ CFU/mL. The Cp cutoff value was 35 cycles. Lysates of M. tuberculosis strains H37Rv and CSU93 showed solid detection by all primers, with estimated cell concentrations in samples of greater than 10⁹ CFU/mL for this material. As the cell number of samples increased, fewer cycles were required to detect signal, as expected, and as indicated by a lower Cp value.

For spiked sputa, which were prepared by spiking cultured bacteria into culture and smear-negative sputa to achieve 60-85 AFB/high power field, the ilvC primer sets shown in Table 10 detected ilvC sequences specifically in 1/10 dilutions of starting material, and at a 1/100 dilution of starting material for all primer pairs other than SEQ ID NOs: 29 and 30, and at a 1/000 dilution of starting material for the primer pairs SEQ ID NOs: 19 and 20 and SEQ ID NOs: 29 and 30. Negative sputa were correctly identified as being negative in all assays.

For clinical smear and culture positive sputa, SAL, and lymph node needle biopsy (FNA), the ilvC primer sets of SEQ ID NOs: 19 and 20 and SEQ ID NOs: 27 and 28 detected ilvC sequences specifically in BAL; the ilvC primer sets of SEQ ID NOs: 27 and 28 and SEQ ID NOs: 29 and 30 detected ilvC sequences specifically in FNA; and the ilvC primer sets shown in Table 10 failed to amplify nucleic acid from sputa comprising M. avium. Table 12 provides Cp values (No. cycles) for all samples. The Cp value is the crossover point, where the specific product crosses over the background signal, measured in cycles.

TABLE 12
Cp values for culture and clinical samples
employing primers of the invention

| | | Primer pair from Table 10 | | | |
|---|---|---|---|---|---|
| Sample | | ilvC1F/ | ilvC2F/ | ilvC3F/ | rtM.tbilvCF/ |
| Source | Concentration | ilvC1R | ilvC2R | ilvC3R | rtM.tbilvCR |
| Culture | | | | | |
| A | 10⁸ CFU/ml | 20.71 | 20.91 | 20.36 | 21.10 |
| A | 10⁶ CFU/ml | 26.76 | 26.75 | 26.34 | 26.78 |
| A | 10⁴ CFU/ml | 33.50 | 33.04 | 29.82 | 34.53 |
| A | 10² CFU/ml | — | — | — | — |
| B | 10⁸ CFU/ml | 20.02 | 19.74 | 19.33 | 20.16 |
| B | 10⁶ CFU/ml | 26.01 | 26.36 | 25.79 | 27.17 |
| B | 10⁴ CFU/ml | 33.58 | 32.79 | 30.06 | 34.03 |
| B | 10² CFU/ml | — | — | — | — |
| H37Rv | | 16.89 | 16.76 | 16.64 | 17.18 |
| Csu93 | | 16.77 | 16.86 | 16.55 | 17.29 |
| Sputum | | | | | |
| RCPA-A | Nil AFB | — | — | — | — |
| RCPA-B | 60-85 | 32.70 | 31.53 | 29.47 | 32.88 |
| | AFB/100HPF | | | | |
| | 10⁻¹ dilution | | | | |
| RCPA-C | 60-85 | — | — | — | — |
| | AFB/100HPF | | | | |
| | 10⁻³ dilution | | | | |
| RCPA-D | Nil AFB | — | — | — | — |
| RCPA-E | 60-85 | 32.36 | 31.46 | 29.52 | 32.65 |
| | AFB/100HPF | | | | |
| | 10⁻¹ dilution | | | | |
| RCPA-F | 60-85 | 36.19 | — | — | 37.20 |
| | AFB/100HPF | | | | |
| | 10⁻³ dilution | | | | |
| RCPA-G | 60-85 | 34.84 | 34.91 | — | 32.88 |
| | AFB/100HPF | | | | |
| | 10⁻² dilution | | | | |
| Mtb +ve | Not known | — | — | — | — |
| M. avium +ve | Not known | — | — | — | — |
| BAL | Not known | 33.04 | — | — | 35.53 |
| FNA | Not known | 35.00 | 35.22 | — | — |

### Example 10

### Nucleic Acid Sequence Based Amplification (NASBA) Employing ilvC Molecular Beacons

This example provides support for extension of the amplification platform(s) described in Examples 2, 8 and 9 hereof to a NASBA-molecular beacon platform.

### 1. Molecular Beacon and Primers

Molecular beacon probes were produced by Biosearch Technologies (Novato, Calif.) and purified by high performance liquid chromatography (HPLC), as described by Vet et al., In: Oligonucleotide synthesis: Methods and Applications (Herdewijn, P. ed.), Humana Press, Totowa, N.J., Vol. 288, pp. 273-290 (2004). A Molecular Beacon designated ilvc4-MB was produced based on the sequence of primer ilvC4F (SEQ ID NO: 33) as follows:

Primer ilvC4F (SEQ ID NO: 33):
AAGACGACGTTCAAAGACGA.

The molecular beacon comprised SEQ ID NO: 33 and complementary 5′-end and 3′-end sequences not present in the flanking ilvC mRNA or gene sequence, as follows:

Beacon ilvc4-MB (SEQ ID NO: 34):
ccgggAAGACGACGTTCAAAGACGAcccgg.

Oligonucleotide primers were produced by Integrated DNA Technologies (Coralville, Iowa). Primers flanking the annealing site of the molecular beacon were produced based on sequence data for M. tuberculosis H37Rv and comprised SEQ ID NO: 31 or SEQ ID NO: 32 (Table 10) or the following sequences:

Primer ilvC5F (SEQ ID NO: 35):
CGTGTTTGGTTGCGGTAGAG;
Primer ilvC6R (SEQ ID NO: 36):
GTTTGCTCACCGAACAGGTC;
and
Primer ilvC7R (SEQ ID NO: 37):
CCGCACAACACCGTTTGCTCACCGAAC.

Reverse primers for use with a molecular beacon included a 3′-tail sequence. For example, a reverse primer was produced based on SEQ ID NO: 32 and having the following sequence, wherein SEQ ID NO: 32 is underlined:

Primer ilvC3R-tail (SEQ ID NO: 38):
AATTCTAATACGACTCACTATAGGGTCACAACACCGTTTGCTCACC.

In another example, a reverse primer was produced based on SEQ ID NO: 36 and having the following sequence, wherein SEQ ID NO: 36 is underlined:

Primer ilvC6R-tail (SEQ ID NO: 39):
AATTCTAATACGACTCACTATAGGGTGTTTGCTCACCGAACAGGTC.

In another example, a reverse primer was produced based on SEQ ID NO: 37 and having the following sequence, wherein SEQ ID NO: 37 is underlined:

Primer ilvC7R-tail (SEQ ID NO: 40:
AATTCTAATACGACTCACTATAGGGTCCGCACAACACCGTTTGCTCACCGAAC.

Combinations of forward and reverse primers for use with the molecular beacon are evident based on the alignment of the primer sequences with the ilvC sequence set forth in SEQ ID NO: 2. For example, Primer set I comprised primer ilvC3F shown in Table 10 (SEQ ID NO: 32) and primer ilvc3R-tail (SEQ ID NO: 38). Primer set II comprised primer ilvC5F (SEQ ID NO: 35) and primer ilvC6R-tail (SEQ ID NO: 39). Primer set III comprised primer ilvC5F (SEQ ID NO: 35) and primer ilvc3R-tail (SEQ ID NO: 38). Primer set IV comprised primer ilvC3F (SEQ ID NO: 32) and primer ilvC6R-tail (SEQ ID NO: 39). Primer set V comprised primer ilvC3F (SEQ ID NO: 32) and primer ilvC7R-tail (SEQ ID NO: 40). Primer set VI comprised primer ilvC5F (SEQ ID NO: 35) and primer ilvC7R-tail (SEQ ID NO: 40).

### 2. Pilot Amplification Reactions on Culture Samples Using Molecular Beacon

Primer sets I-IV and the molecular beacon were assessed by amplification using DNA from the four M.tb positive samples. Briefly, amplifications were performed using a Mx3005P Multiplex Quantitative PCR System (Stratagene, La Jolla, Calif.). For amplification, 20 μl a reactions comprised 1 U of AmpliTaq Gold DNA Polymerase (Applied Biosystems, Foster City, Calif.), 4 mM MgCl₂, 250 μM of each dNTP, and 0.4 μM of each primer, 0.1 μM Beacon in 1×PCR buffer (Applied Biosystems). PCR cycling conditions included an initial denaturation step of 95° C. for 10 min, followed by 40 cycles each comprising 95° C. for 30 s (denaturation) followed by 55° C. for 30 s (annealing) and 72° C. for 30 s (extension).

These preliminary studies produced cycle threshold values for the four primer sets I-IV supra of between 14.96 and 17.81, indicating their suitability for detecting M.tb complex ilvC nucleic acid.

3. Quantitive Assay for ilvC on Culture Samples Using Molecular Beacon

Four M. tb-positive culture samples were obtained and RNA produced as described herein as a template for amplification. Briefly, primer sets I and II and the molecular beacon were assessed by amplification using RNA from the four M.tb positive samples as a template. NASBA reactions were performed using the NucliSENS basic kit version 2 (bioMérieux), with 5 μl reaction volumes comprising reagent mix, 0.2 μM each primer, 0.1 μM molecular beacon, 2.5 μl RNA template, and 2.5 μl NASBA enzyme mix (bioMérieux). The enzyme mixture comprised T7 RNA polymerase, avian myeloblastosis virus (AMV) reverse transcriptase, RNase H, and bovine serum albumin, and the enzyme mixture was added to the reaction mixture after a two-step incubation of 2 min at 65° C. and 2 min at 41° C. NASBA assay reactions were performed using a Stratagene Mx3005P real-time PCR system (Stratagene, La Jolla, Calif.) with the setting of 180 cycles at 41° C. with 30 s for each cycle. The fluorescence signal was measured once for each cycle. The cutoff threshold for the positive signal was set as 15% higher than the end point signal from the no-template control, which used 2.5 μl of nuclease-free water instead of nucleic acids in the reaction mixture. The time to positive was defined as the time point when the positive signal crossed the threshold (shorter time indicates more sensitive detection).

Both primer sets I and II provided positive amplification products i.e., above background, in about 30-60 mins, and primer set II provided significantly higher levels of amplification product within 37-50 mins from these culture samples.

4. Amplification from Synthetic RNA Transcript

To further evaluate the amplification system, a synthetic RNA internal transcript based on the ilvC sequence was produced and employed to determine the sensitivity of the probe/primer combinations. Briefly, a DNA amplicon was produced using a forward primer linked to a T7 promoter sequence. After purification of the DNA amplicon, the synthetic RNA transcript was produced by transcription in vitro using an RNAMaxx High Yield Transcription kit (Stratagene) and then treated by RNase-free DNase I (NEB, Ipswich, Mass.) for 30 min at 37° C. RNA products were purified using the RNeasy kit according to the RNA cleanup protocol (Qiagen). Purified RNA targets were quantified by a Quant-iT RiboGreen RNA quantification kit (Molecular Probes, Invitrogen, Carlsbad, Calif.). The copy numbers of RNA were calculated. Then, sensitivity for ilvC NASBA was evaluated. A dilution series of synthetic RNA targets ranging from 10⁹ to 10² copies was also used to generate an assay standard curve with NASBA being performed as in the preceding section using primer sets I and II. A dilution series of synthetic RNA targets ranging from 10⁵ to 10² copies was also used to generate an assay standard curve with NASBA being performed as in the preceding section using primer sets III, IV, V and VI.

In these experiments, primer sets I and II detected ilvC RNA at levels above background in about 20-60 mins, and primer set II provided significantly higher levels of amplification product in an earlier time frame from these samples. Primer set III detected 10⁵ copies of ilvC RNA at levels above background within 50.9 min and detected 10⁴ copies of ilvC RNA at levels above background in 54.1 min. Primer set V detected 10⁵ copies of ilvC RNA at levels above background within 47.5 min and detected 10⁴ copies of ilvC RNA at levels above background in 49.5 min. These data indicate that primer sets I, II, III and V are useful for specific detection of ilvC mRNA.

5. Amplification from M.tb RNA Transcript

To further evaluate the amplification system, primer sets I and II were also tested using TB RNA samples, under the same conditions as described herein above. Again, primer set II provided higher levels of specific amplicon in an earlier time frame than primer set I.

6. Recovery of RNA Isolated from Cell Culture

RNA was isolated from M.tb cultures scraped from solid media. The quality of the RNA was evaluated by the presence of 23S and 16S bands in the preparations following gel electrophoresis of RNAs. The RNAs were subjected to RT-amplification using primer set II supra alongside a dilution series of synthetic RNA supra as a standard, under assay conditions described above. RNA copy number was determined from standard curves prepared from this study.

In these experiments, the time to product (TTP) for synthetic RNA standard was 22.11 min for a 10⁹ copies of RNA, increasing to 67.66 min for 10⁴ copies. The linear range at least between 10⁹ and 10⁴ copies is acceptable for practical purposes (R²=0.999).

The M.tb culture samples provided specific ilvC amplification products in 35.58 min to 50.35 min, consistent with previous tests. The primer set H was able to detect at least 9.61×10⁴ copies of ilvC RNA under these conditions. Primer set V was able to detect about 10³ copies of ilvC RNA under these conditions.

The detection levels achieved in culture has previously shown to be less sensitive than that achievable in primary sputum specimens. Accordingly, the inventors are isolating and testing amplification sensitivity using primary sputa with the probes and primers herein.

1. A method of specifically detecting the presence of one or more Mycobacteria of the M. tuberculosis complex, said method comprising detecting ilvC nucleic acid of one or more Mycobacteria of the M. tuberculosis complex in a sample under conditions that do not detect ilvC nucleic acid of the M. avium complex.

2. The method according to claim 1, wherein detecting ilvC nucleic acid of one or more Mycobacteria of the M. tuberculosis complex comprises amplifying one or more ilvC nucleic acids to thereby produce amplified ilvC nucleic acid and detecting the amplified nucleic acid, wherein the detection of amplified ilvC nucleic acid is indicative of the presence of one or more of said Mycobacteria of the M. tuberculosis complex in the sample.

3. The method of claim 1, wherein the detected ilvC nucleic acid comprises a sequence of M. tuberculosis ilvC DNA or RNA.

4. The method of claim 1, wherein the M. tuberculosis complex organism is selected from M. tuberculosis, M. bovis, M. africanum, M. canetti and M. microti or a combination thereof.

5. The method of claim 4, wherein the M. tuberculosis complex organism is selected from M. tuberculosis and M. bovis or a combination thereof.

6. The method of claim 4, wherein the M. tuberculosis complex organism is M. tuberculosis.

7. The method according to claim 6, wherein the M. tuberculosis is a clinical strain or clinical isolate of M. tuberculosis.

8. The method of claim 4, wherein the M. tuberculosis complex organism is M. bovis.

9. The method according to claim 1, wherein a Mycobacterium of the M. avium complex is M. avium.

10. The method according to claim 1, wherein a Mycobacterium of the M. avium complex is M. intracellulaire.

11. The method according to claim 1, wherein detecting ilvC nucleic acid of one or more Mycobacteria of the M. tuberculosis complex comprises performing an amplification reaction with thermal cycling.

12. The method according to claim 1, wherein detecting ilvC nucleic acid of one or more Mycobacteria of the M. tuberculosis complex comprises performing an amplification reaction without thermal cycling.

13. The method according to claim 11, wherein the amplification reaction is performed using a single-stranded or double-stranded cDNA template or DNA/RNA hybrid molecule produced by reverse-transcription of RNA.

14. The method according to claim 1, wherein detecting ilvC nucleic acid of one or more Mycobacteria of the M. tuberculosis complex without detecting ilvC nucleic acid of the M. avium complex comprises detecting ilvC nucleic acid comprising a sequence of at least 20 contiguous nucleotides in length of SEQ ID NO: 2 or a homologous sequence thereto from one or more Mycobacteria of the M. tuberculosis complex.

15. The method according to claim 14, comprising detecting ilvC nucleic acid comprising a sequence of at least 40 contiguous nucleotides in length of SEQ ID NO: 2 or a homologous sequence thereto from one or more Mycobacteria of the M. tuberculosis complex.

16. The method according to claim 14, comprising detecting ilvC nucleic acid comprising a sequence of at least 50 contiguous nucleotides in length of SEQ ID NO: 2 or a homologous sequence thereto from one or more Mycobacteria of the M. tuberculosis complex.

17. The method according to claim 1, wherein detecting ilvC nucleic acid of one or more Mycobacteria of the M. tuberculosis complex comprises amplifying one or more ilvC nucleic acids to thereby produce amplified ilvC nucleic acid and detecting the amplified nucleic acid and wherein said amplifying produces an amplicon of at least 50 contiguous nucleotides in length of SEQ ID NO: 2 or a homologous sequence thereto from one or more Mycobacteria of the M. tuberculosis complex.

18. The method according to claim 17, wherein the amplicon comprises at least 80 contiguous nucleotides in length of SEQ ID NO: 2 or a homologous sequence thereto from one or more Mycobacteria of the M. tuberculosis complex.

19. The method according to claim 1, wherein detecting ilvC nucleic acid of one or more Mycobacteria of the M. tuberculosis complex without detecting ilvC nucleic acid of the M. avium complex comprises performing an amplification reaction using one or a plurality of primers that each comprise a sequence of at least about 18 contiguous nucleotides of SEQ ID NO: 2 from position 420 to position 600 thereof, or a sequence complementary to SEQ ID NO: 2 from position 420 to position 600 thereof.

20. The method according to claim 1, wherein detecting ilvC nucleic acid of one or more Mycobacteria of the M. tuberculosis complex without detecting ilvC nucleic acid of the M. avium complex comprises performing an amplification reaction using one or a plurality of primers that each comprise a sequence of at least about 18 contiguous nucleotides of SEQ ID NO: 2 from position 40 to position 180 thereof, or a sequence complementary to SEQ ID NO: 2 from position 40 to position 180 thereof.

21. The method according to claim 1, wherein detecting ilvC nucleic acid of one or more Mycobacteria of the M. tuberculosis complex without detecting ilvC nucleic acid of the M. avium complex comprises performing an amplification reaction using one or a plurality of primers that each comprise a sequence of at least about 18 contiguous nucleotides of SEQ ID NO: 2 from position 880 to position 1000 thereof, or a sequence complementary to SEQ ID NO: 2 from position 880 to position 1000 thereof.

22. The method according to claim 1, wherein detecting ilvC nucleic acid of one or more Mycobacteria of the M. tuberculosis complex without detecting ilvC nucleic acid of the M. avium complex comprises performing an amplification reaction for less than 30 amplification cycles.

23. The method according to claim 22, wherein detecting ilvC nucleic acid of one or more Mycobacteria of the M. tuberculosis complex without detecting ilvC nucleic acid of the M. avium complex comprises performing an amplification reaction for about 12 amplification cycles to about 27 amplification cycles.

24. The method according to claim 22, wherein detecting ilvC nucleic acid of one or more Mycobacteria of the M. tuberculosis complex without detecting ilvC nucleic acid of the M. avium complex comprises performing an amplification reaction for about 14 amplification cycles to about 20 amplification cycles.

25. The method according to claim 1, wherein detecting ilvC nucleic acid of one or more Mycobacteria of the M. tuberculosis complex without detecting ilvC nucleic acid of the M. avium complex comprises performing an amplification reaction on less than about 2 ng/ml input prokaryotic nucleic acid.

26. The method according to claim 1, wherein detecting ilvC nucleic acid of one or more Mycobacteria of the M. tuberculosis complex without detecting ilvC nucleic acid of the M. avium complex comprises performing an amplification reaction using one or a plurality of amplification primers and a labelled probe comprising a sequence capable of hybridizing to a nucleic acid product of the amplification primers to thereby produce a detectable signal.

27. The method according to claim 1, wherein detecting ilvC nucleic acid of one or more Mycobacteria of the M. tuberculosis complex without detecting ilvC nucleic acid of the M. avium complex comprises performing an amplification reaction using one or a plurality of amplification primers and a labelled probe capable of binding to at least one of the amplification primers.

28. The method according to claim 1, wherein the sample comprises a sample selected from the group consisting of cultured M. tuberculosis cells, sputum, broncho alveolar lavage (BAL), a lymph node biopsy, blood, serum, plasma, a fraction of blood, a fraction of serum, a fraction of plasma and urine.

29-33. (canceled)

34. The method according to claim 1, wherein detecting ilvC nucleic acid of one or more Mycobacteria of the M. tuberculosis complex without detecting ilvC nucleic acid of the M. avium complex comprises performing an amplification reaction under conditions sufficient to detect at least about 10⁴ copies of ilvC nucleic acid in less than about 60 minutes.

35. The method according to claim 1, wherein detecting ilvC nucleic acid of one or more Mycobacteria of the M. tuberculosis complex without detecting ilvC nucleic acid of the M. avium complex comprises performing an amplification reaction under conditions sufficient to detect at least about 10³ copies of ilvC nucleic acid in less than about 60 minutes.

36. The method according to claim 1, wherein detecting ilvC nucleic acid of one or more Mycobacteria of the M. tuberculosis complex without detecting ilvC nucleic acid of the M. avium complex comprises performing an amplification reaction under conditions sufficient to detect at least about 10⁴ CFU/ml Mycobacteria of the M. tuberculosis complex.

37. The method according to claim 1 further comprising detecting one or more nucleic acids of one or more Mycobacteria of the M. tuberculosis complex in a sample wherein the one or more nucleic acids are other than ilvC nucleic acid.

38. The method according to claim 37, wherein the one or more nucleic acids other than ilvC nucleic acid is 16s rRNA.

39. The method according to claim 37, wherein the one or more nucleic acids other than ilvC nucleic acid encodes a protein selected from the group consisting of BSX, S9, Rv1265, EF-Tu, P5CR, TetR-like protein and glutamine synthetase or nucleic acid complementary thereto.

40. The method according to claim 39, comprising performing polymerase chain reaction (PCR) to thereby detect nucleic acid comprising a sequence of at least 20 contiguous nucleotides in length of a sequence selected from the group consisting of SEQ ID NOs: 4, 6, 8, 10, 12, 14 and 16.

41. The method according to claim 37, comprising performing an amplification reaction to thereby produce an amplicon of at least 50 contiguous nucleotides in length of the one or more nucleic acids other than ilvC nucleic acid when said nucleic acid is present in the sample.

42. A process for diagnosing an infection by one or more Mycobacteria of the M. tuberculosis complex in a subject, said process comprising performing the method according to claim 1 on a biological sample from a subject to thereby detect ilvC nucleic acid of one or more Mycobacteria of the M. tuberculosis complex in a sample under conditions that do not detect ilvC nucleic acid of the M. avium complex wherein the detection of one or more ilvC nucleic acids of one or more Mycobacteria of the M. tuberculosis complex is indicative of infection.

43. The process according to claim 42, wherein the infection is an active infection.

44. The process according to claim 42, wherein the infection is a latent infection.

45. A process for diagnosing tuberculosis in a subject, said process comprising performing the method according to claim 1 on a biological sample from a subject to thereby detect ilvC nucleic acid of one or more Mycobacteria of the M. tuberculosis complex in a sample under conditions that do not detect ilvC nucleic acid of the M. avium complex wherein the detection of one or more ilvC nucleic acids of one or more Mycobacteria of the M. tuberculosis complex is indicative of tuberculosis.

46. The process according to claim 45, wherein the tuberculosis is pulmonary tuberculosis.

47. The process according to claim 45, wherein the tuberculosis is extrapulmonary tuberculosis.

48. The process according to claim 45 further comprising diagnosing one or more clinical symptoms of tuberculosis in the subject.

49. The process according to claim 45 further comprising diagnosing one or more clinical symptoms of immune suppression in the subject.

50. The process according to claim 45 further comprising diagnosing HIV infection in the subject.

51. A process for treating tuberculosis or infection by one or more mycobacteria of the M. tuberculosis complex, said process comprising:
(i) performing the method according to claim 1 on a sample from a subject to thereby detect one or more mycobacteria of the M. tuberculosis complex in the sample; and
(ii) administering a therapeutically effective amount of a pharmaceutical composition to the subject to thereby reduce the number of pathogenic bacilli in the lung, blood or lymph system of the subject.

52. The method according to claim 12, wherein the amplification reaction is performed using a single-stranded or double-stranded cDNA template or DNA/RNA hybrid molecule produced by reverse-transcription of RNA.
